SEQUENCE LISTING (80 sequences)

SEQ ID NO: 1 - DNA, 5230 bp, synthetic construct
  misc_feature 1..5230: note = Synthetic Polynucleotide
  source 1..5230: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtctagcaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctgactttcatctgtactacattcaagagatgtagtacagatgaaagtctttttctagtctgcaggaagacaatagcgtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 2 - DNA, 4922 bp, synthetic construct
  misc_feature 1..4922: note = Synthetic Polynucleotide
  source 1..4922: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 3 - DNA, 5075 bp, synthetic construct
  misc_feature 1..5075: note = Synthetic Polynucleotide
  source 1..5075: mol_type = other DNA
cgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcagattgtcttcctgcagactagttatgcggcatcagagcagattgtactgagagtgcaccataggggatcgggagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaattgctagtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggcctta

SEQ ID NO: 4 - DNA, 5075 bp, synthetic construct
  misc_feature 1..5075: note = Synthetic Polynucleotide
  source 1..5075: mol_type = other DNA
cgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcagtagcaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctcccgatcccctatggtgcactctcagtacaatctgctctgatgccgcataactagtctgcaggaagacaatgtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggcctta

SEQ ID NO: 5 - DNA, 5079 bp, synthetic construct
  misc_feature 1..5079: note = Synthetic Polynucleotide
  source 1..5079: mol_type = other DNA
tagcaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctgactttcatctgtactacattcaagagatgtagtacagatgaaagtctttttctagtctgcaggaagacaatagccgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggcctta

SEQ ID NO: 6 - DNA, 5082 bp, synthetic construct
  misc_feature 1..5082: note = Synthetic Polynucleotide
  source 1..5082: mol_type = other DNA
gtcgctattgtcttcctgcagactagaaaaagactttcatctgtactacatctcttgaatgtagtacagatgaaagtcagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaattgctacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggcctta

SEQ ID NO: 7 - DNA, 4774 bp, synthetic construct
  misc_feature 1..4774: note = Synthetic Polynucleotide
  source 1..4774: mol_type = other DNA
cgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggcctta

SEQ ID NO: 8 - DNA, 5109 bp, synthetic construct
  misc_feature 1..5109: note = Synthetic Polynucleotide
  source 1..5109: mol_type = other DNA
ctgcgcgctcgctcgctcactgaggccgcccgggcaaagcccgggcgtcgggcgaccttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctcccgatcccctatggtgcactctcagtacaatctgctctgatgccgcataactagtctgcaggaagacaatttaggtagataagtagcatggcgggttaatcattaactacaaggaacccctagtgatggagttggtaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 9 - DNA, 5172 bp, synthetic construct
  misc_feature 1..5172: note = Synthetic Polynucleotide
  source 1..5172: mol_type = other DNA
cgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcagaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgcgggatcccgcttacgctgagtacttcgattcaagagatcgaagtactcagcgtaagttttttccaaagtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggcctta

SEQ ID NO: 10 - DNA, 5172 bp, synthetic construct; identical to SEQ ID NO: 9
  misc_feature 1..5172: note = Synthetic Polynucleotide
  source 1..5172: mol_type = other DNA

SEQ ID NO: 11 - DNA, 5081 bp, synthetic construct
  misc_feature 1..5081: note = Synthetic Polynucleotide
  source 1..5081: mol_type = other DNA
tagcaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctgactttcatctgtactacattcaagagatgtagtacagatgaaagtctttttctagtctgcaggaagacaatagccgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccagatatctattttagatatctggcagaatcccgaaacgggattctggcctcagtgagcgagcgagcgcgcagttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggcctta

SEQ ID NO: 12 - DNA, 5041 bp, synthetic construct
  misc_feature 1..5041: note = Synthetic Polynucleotide
  source 1..5041: mol_type = other DNA
cgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcagtagcaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctcccgatcccctatggtgcactctcagtacaatctgctctgatgccgcataactagtctgcaggaagacaatgtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccttcaagagaggcctcagtgagcgagcgagcgcgcagttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggcctta

SEQ ID NO: 13 - DNA, 5077 bp, synthetic construct
  misc_feature 1..5077: note = Synthetic Polynucleotide
  source 1..5077: mol_type = other DNA
cgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcagtagcaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctcccgatcccctatggtgcactctcagtacaatctgctctgatgccgcataactagtctgcaggaagacaatgtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggcctactttcagtttctgaaagtaggcacatgatgtaaaacatcatgtggcctcagtgagcgagcgagcgcgcagttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggcctta

SEQ ID NO: 14 - DNA, 5077 bp, synthetic construct
  misc_feature 1..5077: note = Synthetic Polynucleotide
  source 1..5077: mol_type = other DNA
cgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcagtagcaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctcccgatcccctatggtgcactctcagtacaatctgctctgatgccgcataactagtctgcaggaagacaatgtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccagatatctattttagatatctggcagaatcccgaaacgggattctggcctcagtgagcgagcgagcgcgcagttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggcctta

SEQ ID NO: 15 - DNA, 5045 bp, synthetic construct
  misc_feature 1..5045: note = Synthetic Polynucleotide
  source 1..5045: mol_type = other DNA
tagcaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctgactttcatctgtactacattcaagagatgtagtacagatgaaagtctttttctagtctgcaggaagacaatagccgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccttcaagagaggcctcagtgagcgagcgagcgcgcagttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggcctta

SEQ ID NO: 16 - DNA, 5081 bp, synthetic construct
  misc_feature 1..5081: note = Synthetic Polynucleotide
  source 1..5081: mol_type = other DNA
tagcaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctgactttcatctgtactacattcaagagatgtagtacagatgaaagtctttttctagtctgcaggaagacaatagccgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggcctactttcagtttctgaaagtaggcacatgatgtaaaacatcatgtggcctcagtgagcgagcgagcgcgcagttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggcctta

SEQ ID NO: 17 - DNA, 5138 bp, synthetic construct
  misc_feature 1..5138: note = Synthetic Polynucleotide
  source 1..5138: mol_type = other DNA
cgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcagaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgcgggatcccgcttacgctgagtacttcgattcaagagatcgaagtactcagcgtaagttttttccaaagtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccttcaagagaggcctcagtgagcgagcgagcgcgcagttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggcctta

SEQ ID NO: 18 - DNA, 5174 bp, synthetic construct
  misc_feature 1..5174: note = Synthetic Polynucleotide
  source 1..5174: mol_type = other DNA
cgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcagaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgcgggatcccgcttacgctgagtacttcgattcaagagatcgaagtactcagcgtaagttttttccaaagtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggcctactttcagtttctgaaagtaggcacatgatgtaaaacatcatgtggcctcagtgagcgagcgagcgcgcagttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggcctta

SEQ ID NO: 19 - DNA, 5174 bp, synthetic construct
  misc_feature 1..5174: note = Synthetic Polynucleotide
  source 1..5174: mol_type = other DNA
cgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcagaattccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgcgggatcccgcttacgctgagtacttcgattcaagagatcgaagtactcagcgtaagttttttccaaagtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccagatatctattttagatatctggcagaatcccgaaacgggattctggcctcagtgagcgagcgagcgcgcagttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggcctta

SEQ ID NO: 20 - DNA, 31 bp, synthetic construct
  misc_feature 1..31: note = Synthetic Polynucleotide
  source 1..31: mol_type = other DNA
cgcctcgagaaattgaagaagatctgttaac

SEQ ID NO: 21 - DNA, 31 bp, synthetic construct
  misc_feature 1..31: note = Synthetic Polynucleotide
  source 1..31: mol_type = other DNA
cgcgcggccgctcttctctggaggggactgt

SEQ ID NO: 22 - DNA, 20 bp, synthetic construct
  misc_feature 1..20: note = Synthetic Polynucleotide
  source 1..20: mol_type = other DNA
gtccaggttgaatcacgggt

SEQ ID NO: 23 - DNA, 20 bp, synthetic construct
  misc_feature 1..20: note = Synthetic Polynucleotide
  source 1..20: mol_type = other DNA
aggatcctgcaaggtcaagc

SEQ ID NO: 24 - DNA, 22 bp, synthetic construct
  misc_feature 1..22: note = Synthetic Polynucleotide
  source 1..22: mol_type = other DNA
atgccaacacagtgctgtctgg

SEQ ID NO: 25 - DNA, 22 bp, synthetic construct
  misc_feature 1..22: note = Synthetic Polynucleotide
  source 1..22: mol_type = other DNA
tgcttgctgatccacatctgct

SEQ ID NO: 26 - DNA, 20 bp, synthetic construct
  misc_feature 1..20: note = Synthetic Polynucleotide
  source 1..20: mol_type = other DNA
gcagcttttagagcagaagt

SEQ ID NO: 27 - DNA, 23 bp, synthetic construct
  misc_feature 1..23: note = Synthetic Polynucleotide
  source 1..23: mol_type = other DNA
tctagttgccagccatctgttgt

SEQ ID NO: 28 - DNA, 20 bp, synthetic construct
  misc_feature 1..20: note = Synthetic Polynucleotide
  source 1..20: mol_type = other DNA
agcaaagaccccaacgagaa

SEQ ID NO: 29 - DNA, 15 bp, synthetic construct
  misc_feature 1..15: note = Synthetic Polynucleotide
  source 1..15: mol_type = other DNA
ggcggcggtcacgaa

SEQ ID NO: 30 - DNA, 22 bp, synthetic construct
  misc_feature 1..22: note = Synthetic Polynucleotide
  source 1..22: mol_type = other DNA
  modified_base 1: mod_base = OTHER; note = Modified with 6-carboxyfluorescein
  modified_base 22: mod_base = OTHER; note = Modified with Carboxytetramethylrhodamine
cgcgatcacatggtcctgctgg

SEQ ID NO: 31 - DNA, 19 bp, synthetic construct
  misc_feature 1..19: note = Synthetic Polynucleotide
  source 1..19: mol_type = other DNA
gactttcatctgtactaca

SEQ ID NO: 32 - DNA, 26 bp, synthetic construct
  misc_feature 1..26: note = Synthetic Polynucleotide
  source 1..26: mol_type = other DNA
ctctgtatcgttccaattttagtata

SEQ ID NO: 33 - DNA, 52 bp, synthetic construct
  misc_feature 1..52: note = Synthetic Polynucleotide
  source 1..52: mol_type = other DNA
gactttcatctgtactacattcaagagatgtagtacagatgaaagtcttttt

SEQ ID NO: 34 - DNA, 54 bp, synthetic construct
  misc_feature 1..54: note = Synthetic Polynucleotide
  source 1..54: mol_type = other DNA
gcttacgctgagtacttcgattcaagagatcgaagtactcagcgtaagtttttt

SEQ ID NO: 35 - DNA, 47 bp, synthetic construct
  misc_feature 1..47: note = Synthetic Polynucleotide
  source 1..47: mol_type = other DNA
tcctttcagatgtcataacttcaagagatgtagtacagatgaaagtc

SEQ ID NO: 36 - DNA, 47 bp, synthetic construct
  misc_feature 1..47: note = Synthetic Polynucleotide
  source 1..47: mol_type = other DNA
tcctttcagatgtcatacattcaagagatgtagtacagatgaaagtc

SEQ ID NO: 37 - DNA, 47 bp, synthetic construct
  misc_feature 1..47: note = Synthetic Polynucleotide
  source 1..47: mol_type = other DNA
tcctttcatctgtcataacttcaagagatgtagtacagatgaaagtc

SEQ ID NO: 38 - DNA, 47 bp, synthetic construct
  misc_feature 1..47: note = Synthetic Polynucleotide
  source 1..47: mol_type = other DNA
gactttcagatgtcataacttcaagagatgtagtacagatgaaagtc

SEQ ID NO: 39 - DNA, 47 bp, synthetic construct
  misc_feature 1..47: note = Synthetic Polynucleotide
  source 1..47: mol_type = other DNA
tcctttcagatgtactacattcaagagatgtagtacagatgaaagtc

SEQ ID NO: 40 - DNA, 47 bp, synthetic construct
  misc_feature 1..47: note = Synthetic Polynucleotide
  source 1..47: mol_type = other DNA
gactggcagatgtactacattcaagagatgtagtacagatgaaagtc

SEQ ID NO: 41 - DNA, 47 bp, synthetic construct
  misc_feature 1..47: note = Synthetic Polynucleotide
  source 1..47: mol_type = other DNA
tcctttcatctgtcatacattcaagagatgtagtacagatgaaagtc

SEQ ID NO: 42 - DNA, 47 bp, synthetic construct
  misc_feature 1..47: note = Synthetic Polynucleotide
  source 1..47: mol_type = other DNA
gactttcagatgtcatacattcaagagatgtagtacagatgaaagtc

SEQ ID NO: 43 - DNA, 47 bp, synthetic construct
  misc_feature 1..47: note = Synthetic Polynucleotide
  source 1..47: mol_type = other DNA
tcctttcatctgtactaacttcaagagatgtagtacagatgaaagtc

SEQ ID NO: 44 - DNA, 47 bp, synthetic construct
  misc_feature 1..47: note = Synthetic Polynucleotide
  source 1..47: mol_type = other DNA
gactggcatctgtactaacttcaagagatgtagtacagatgaaagtc

SEQ ID NO: 45 - DNA, 47 bp, synthetic construct
  misc_feature 1..47: note = Synthetic Polynucleotide
  source 1..47: mol_type = other DNA
gactttcagatgtactaacttcaagagatgtagtacagatgaaagtc

SEQ ID NO: 46 - DNA, 47 bp, synthetic construct
  misc_feature 1..47: note = Synthetic Polynucleotide
  source 1..47: mol_type = other DNA
gactttcatctgtcataacttcaagagatgtagtacagatgaaagtc

SEQ ID NO: 47 - DNA, 47 bp, synthetic construct
  misc_feature 1..47: note = Synthetic Polynucleotide
  source 1..47: mol_type = other DNA
gactttcatctgtactacattcaagagatgtagtacagatgaaagag

SEQ ID NO: 48 - DNA, 47 bp, synthetic construct
  misc_feature 1..47: note = Synthetic Polynucleotide
  source 1..47: mol_type = other DNA
ctctttcatctgtactacattcaagagatgtagtacagatgaaagtc

SEQ ID NO: 49 - DNA, 47 bp, synthetic construct
  misc_feature 1..47: note = Synthetic Polynucleotide
  source 1..47: mol_type = other DNA
tcctttcatctgtactacattcaagagatgtagtacagatgaaagtc

SEQ ID NO: 50 - DNA, 47 bp, synthetic construct
  misc_feature 1..47: note = Synthetic Polynucleotide
  source 1..47: mol_type = other DNA
gactggcatctgtactacattcaagagatgtagtacagatgaaagtc

SEQ ID NO: 51 - DNA, 47 bp, synthetic construct
  misc_feature 1..47: note = Synthetic Polynucleotide
  source 1..47: mol_type = other DNA
gactttcagatgtactacattcaagagatgtagtacagatgaaagtc

SEQ ID NO: 52 - DNA, 47 bp, synthetic construct
  misc_feature 1..47: note = Synthetic Polynucleotide
  source 1..47: mol_type = other DNA
gactttcatctgtcatacattcaagagatgtagtacagatgaaagtc

SEQ ID NO: 53 - DNA, 47 bp, synthetic construct
  misc_feature 1..47: note = Synthetic Polynucleotide
  source 1..47: mol_type = other DNA
gactttcatctgtactaacttcaagagatgtagtacagatgaaagtc

SEQ ID NO: 54 - DNA, 47 bp, synthetic construct
  misc_feature 1..47: note = Synthetic Polynucleotide
  source 1..47: mol_type = other DNA
gactttcatctgtactacattcaagagatgtagtacagatgaaagtc

SEQ ID NO: 55 - DNA, 52 bp, synthetic construct; identical to SEQ ID NO: 33
  misc_feature 1..52: note = Synthetic Polynucleotide
  source 1..52: mol_type = other DNA

SEQ ID NO: 56 - DNA, 58 bp, synthetic construct
  misc_feature 1..58: note = Synthetic Polynucleotide
  source 1..58: mol_type = other DNA
gactttcatctgtactacaaagttcaagagactttgtagtacagatgaaagtcttttt

SEQ ID NO: 57 - RNA, 71 nt, synthetic construct
  misc_feature 1..71: note = Synthetic Polynucleotide
  source 1..71: mol_type = other RNA
gcgactgtaaacatcctcgactggaagctgtgaagccacaaatgggctttcagtcggatgtttgcagctgc

SEQ ID NO: 58 - DNA, 92 bp, synthetic construct
  misc_feature 1..92: note = Synthetic Polynucleotide
  misc_difference 1..3: note = n is a, c, g, or t
  misc_difference 85..87: note = n is a, c, g, or t
  source 1..92: mol_type = other DNA
nnnagtgagcgacctttgtagtacagatgaaagtcctgtgaagccacaagtagggactttcatctactacaaaggctgcccactnnnttttt

SEQ ID NO: 59 - DNA, 100 bp, synthetic construct
  misc_feature 1..100: note = Synthetic Polynucleotide
  source 1..100: mol_type = other DNA
cagatctgactttcatctgtactacattcgctcactgaggccgggcgaccaaaggtcgcccgacgcccgggctttgcccgggcggcctcagtgagcgagc

SEQ ID NO: 60 - DNA, 73 bp, synthetic construct
  misc_feature 1..73: note = Synthetic Polynucleotide
  source 1..73: mol_type = other DNA
cagatctgactttcatctgtactacaggtcgcccgacgcccgggctttgcccgggcggcctcagtgagcgagc

SEQ ID NO: 61 - DNA, 86 bp, synthetic construct
  misc_feature 1..86: note = Synthetic Polynucleotide
  source 1..86: mol_type = other DNA
cagatctgactttcatctgtactacattcaccgggcaaagcccgggcgtcgggcgacctttgcccgggcggcctcagtgagcgagc

SEQ ID NO: 62 - DNA, 39 bp, synthetic construct
  misc_feature 1..39: note = Synthetic Polynucleotide
  source 1..39: mol_type = other DNA
cagatctgactttcatccgggcggcctcagtgagcgagc

SEQ ID NO: 63 - DNA, 43 bp, synthetic construct
  misc_feature 1..43: note = Synthetic Polynucleotide
  source 1..43: mol_type = other DNA
cagatctgactttcatctgtactagcggcctcagtgagcgagc

SEQ ID NO: 64 - DNA, 46 bp, synthetic construct
  misc_feature 1..46: note = Synthetic Polynucleotide
  source 1..46: mol_type = other DNA
cagatctgactttcatctgtactattcgcggcctcagtgagcgagc

SEQ ID NO: 65 - DNA, 81 bp, synthetic construct
  misc_feature 1..81: note = Synthetic Polynucleotide
  source 1..81: mol_type = other DNA
cagatctgactttcatctgtactacatctccaaaggtcgcccgacgcccgggctttgcccgggcggcctcagtgagcgagc

SEQ ID NO: 66 - DNA, 101 bp, synthetic construct
  misc_feature 1..101: note = Synthetic Polynucleotide
  source 1..101: mol_type = other DNA
ctcgctcactgaggccgcccgggcaaagcccgggcgtcgggcgacctttggtcgcccggcctcagtgtagtacagatgaaagtctttttctagtctgcagg

SEQ ID NO: 67 - DNA, 48 bp, synthetic construct
  misc_feature 1..48: note = Synthetic Polynucleotide
  source 1..48: mol_type = other DNA
ctcgctcactgaggccgccccgatgaaagtctttttctagtctgcagg

SEQ ID NO: 68 - DNA, 61 bp, synthetic construct
  misc_feature 1..61: note = Synthetic Polynucleotide
  source 1..61: mol_type = other DNA
ctcgctcactgaggccggggcgacttgtagtacagatgaaagtctttttctagtctgcagg

SEQ ID NO: 69 - DNA, 59 bp, synthetic construct
  misc_feature 1..59: note = Synthetic Polynucleotide
  source 1..59: mol_type = other DNA
ctcgctcactgaggccgccgagatgtagtacagatgaaagtctttttctagtctgcagg

SEQ ID NO: 70 - DNA, 84 bp, synthetic construct
  misc_feature 1..84: note = Synthetic Polynucleotide
  source 1..84: mol_type = other DNA
ctcgctcactgaggccgcccgggcaaagcccgggcgtcgggcgaccaatgtagtacagatgaaagtctttttctagtctgcagg

SEQ ID NO: 71 - DNA, 24 bp, synthetic construct
  misc_feature 1..24: note = Synthetic Polynucleotide
  source 1..24: mol_type = other DNA
ctcgctctttttctagtctgcagg

SEQ ID NO: 72 - DNA, 130 bp, synthetic construct
  misc_feature 1..130: note = Synthetic Polynucleotide
  source 1..130: mol_type = other DNA
gacgcgcgagcgagcgagtgactccggcccgctggtttccagcgggctgcgggcccgaaacgggcccgccggagtcactcgctcgctcgcgcgtctctccctcaccggttgaggtagtgatccccaagga

SEQ ID NO: 73 - DNA, 87 bp, synthetic construct
  misc_feature 1..87: note = Synthetic Polynucleotide
  source 1..87: mol_type = other DNA
gacccccgcccattgacgtcaataatgacgtatgttcccatagtaacgccaatagggactttccattcacgtcaatgggtggactat

SEQ ID NO: 74 - DNA, 107 bp, synthetic construct
  misc_feature 1..107: note = Synthetic Polynucleotide
  source 1..107: mol_type = other DNA
ctttccattgacgtcaatgggtggagtatttacggtaaactgcccacttggcagtacatcaagtgtacatttagccaagtacgccccctattgacgtcaatgacggt

SEQ ID NO: 75 - DNA, 90 bp, synthetic construct
  misc_feature 1..90: note = Synthetic Polynucleotide
  source 1..90: mol_type = other DNA
catctcccccccctccccacccccaattttgtatttatttattttttaattattttgtgcagcgatgggggcgggggggggggggcgcgc

SEQ ID NO: 76 - DNA, 93 bp, synthetic construct
  misc_feature 1..93: note = Synthetic Polynucleotide
  source 1..93: mol_type = other DNA
tgtccggcgagggcgagggcgatgccacctacggcaagctgaccctgaagttcatctgcaccaccggcaagctgcccgtgccctggcccaccc

SEQ ID NO: 77 - DNA, 105 bp, synthetic construct
  misc_feature 1..105: note = Synthetic Polynucleotide
  source 1..105: mol_type = other DNA
gcctggcgcgcgcccccccccctccccacccccaattttgtatttatttattttttaattattttgtgcagcgatgggggcgggggggggggggcgcgcgccagg

SEQ ID NO: 78 - DNA, 104 bp, synthetic construct
  misc_feature 1..104: note = Synthetic Polynucleotide
  source 1..104: mol_type = other DNA
ttaccgtaaatactccgcccattgacgtcaataatgacgtatgttcccatagtaacgccaatgggactttccattgacgtcaatgggtggagtatttacggtaa

SEQ ID NO: 79 - DNA, 119 bp, synthetic construct
  misc_feature 1..119: note = Synthetic Polynucleotide
  source 1..119: mol_type = other DNA
ccatttaccgtcattgacgtcaatgggtggagtatttacggtaaactgcccacttggcagtacatcaagtgtacatttagccaagtacgccccctattgacgtcaatgacggtaaatgg

SEQ ID NO: 80 - DNA, 101 bp, synthetic construct
  misc_feature 1..101: note = Synthetic Polynucleotide
  source 1..101: mol_type = other DNA
acgagggtgggcgagggcgagggcgatgccacctacggcaagctgaccctgaagttcatctgcaccaccggcaagctgcccgtgccctggcccaccctcgt

(19) United States
(12) Patent Application Publication
(10) Pub. No.: US 2024/0409940 A1
(43) Pub. Date: Dec. 12, 2024

(54) MODIFIED AAV CONSTRUCTS AND USES THEREOF

(71) Applicant: University of Massachusetts, Boston, MA (US)

(72) Inventors: Guangping Gao, Worcester, MA (US); Jun Xie, Worcester, MA (US); Phillip D. Zamore, Worcester, MA (US)

(73) Assignee: University of Massachusetts, Boston, MA (US)

(21) Appl. No.: 18/746,105

(22) Filed: Jun. 18, 2024

Related U.S. Application Data
(63) Continuation of application No. 17/316,769, filed on May 11, 2021, now Pat. No. 12,054,715.
Application No. 17/316,769 is a continuation of application No. 15/568,650, filed on Oct. 23, 2017, now Pat. No. 11,046,955.
(60) Provisional application No. 62/152,602, filed on Apr. 24, 2015.

(51) Int. Cl. C12N 15/113; A61K 31/713; C12N 15/11; C12N 15/86

(52) U.S. Cl. CPC C12N 15/113; A61K 31/713; C12N 15/111; C12N 15/86; C12N 2310/11; C12N 2310/141; C12N 2310/531; C12N 2330/51; C12N 2750/14143; C12N 2750/14171

(57) ABSTRACT

The present disclosure relates to the field of rAAV delivery of transgenes. In some aspects, the disclosure relates to RNAi. Provided herein are recombinant adeno-associated virus (rAAV) vectors comprising modified ITRs. In some embodiments, the modified ITRs comprise a sequence encoding a shRNA, miRNA, or AmiRNA.

### RELATED APPLICATIONS

This Application is a continuation of U.S. application Ser. No. 17/316,769, filed May 11, 2021, which is a continuation of U.S. application Ser. No. 15/568,650, filed Oct. 23, 2017, which is a national stage Application under 35 U.S.C. 371 of International Patent Application Serial No. PCT/US2016/027848, filed Apr. 15, 2016, entitled “MODIFIED AAV CONSTRUCTS AND USES THEREOF”, which claims the benefit under 35 USC 119(e)) of U.S. Provisional Application Ser. No. 62/152,602, filed Apr. 24, 2015, entitled “MODIFIED AAV CONSTRUCTS AND USES THEREOF”, the entire contents of each of which are incorporated herein by reference.

### REFERENCE TO AN ELECTRONIC SEQUENCE LISTING

The contents of the electronic sequence listing (U012070068US03-SEQ-LJG.xml; Size: 202,924 bytes; and Date of Creation: Jun. 13, 2024) is herein incorporated by reference in its entirety.

### FIELD OF THE INVENTION

Some aspects of the invention relate to the field of gene expression constructs. Some aspects of the invention relate to viral expression constructs, for example, adeno-associated virus (AAV)-related expression constructs. Some aspects of the invention relate to the field of RNAi.

### BACKGROUND OF INVENTION

Recombinant AAV (rAAV) vectors are useful for the delivery of transgenes into a variety of cell types and tissues. In particular, rAAV vector-delivered RNAi molecules (e.g., shRNA, miRNA, and AmiRNA) are a valuable tool for gene function studies and have many gene therapy applications. For example, shRNA cassettes can be cloned into rAAV vector genomes to achieve a high efficacy of gene silencing in vivo. However, the replication and packing efficiency of rAAV vectors containing nucleic acids encoding hairpin-forming RNA cassettes is significantly lower than rAAV vectors without hairpin-forming RNA cassettes. Accordingly, methods and compositions that increase the replication and packaging efficiency of rAAV vectors containing hairpin-forming RNA cassettes is needed.

### SUMMARY OF INVENTION

rAAV vector-delivered RNAi molecules are a valuable tool for gene function studies and have many gene therapy applications. In some embodiments, microRNA (miRNA) and artificial miRNA (AmiRNA) are useful therapeutic molecules because they overcome cellular toxicity issues related to the saturation of RNAi machinery by short-hairpin RNA (shRNA). However, in some cases, introduction of nucleic acid sequences encoding hairpin-forming RNA (e.g., shRNA, miRNA, and AmiRNA) may have deleterious effects on rAAV genome replication and rAAV yield, resulting in the generation of a heterogeneous population of rAAVs having either full length or truncated vector genomes.

The instant disclosure provides compositions and methods that overcome these issues and allow efficient, safe and sustained in vivo gene silencing. The instant invention is based, in part, on a surprising discovery that DNA fragments encoding RNA hairpin structures (e.g., shRNA, miRNA, and AmiRNA) can serve a function similar to a mutant inverted terminal repeat (ITR) during viral genome replication, generating self-complementary vector genomes.

Accordingly, in some aspects, the disclosure provides an rAAV vector comprising a single-stranded self-complementary nucleic acid with inverted terminal repeats (ITRs) at each of two ends and an inner portion comprising a hairpin-forming nucleic acid.

In some aspects, the disclosure provides an isolated nucleic acid having one inverted terminal repeat at a first terminus and a promoter operably linked with a sequence encoding a hairpin-forming RNA at a second terminus, wherein the isolated nucleic acid is configured for forming a self-complementary AAV (scAAV) vector.

In some embodiments, an isolated nucleic acid is present on a plasmid. Plasmids can be circular plasmids or linearized plasmids.

In some embodiments, hairpin-forming nucleic acid comprises a sequence encoding an hairpin-forming RNA. In some embodiments, sequence encoding the hairpin-forming RNA is operably linked with a promoter.

In some embodiments, hairpin-forming nucleic acid is substituted at a position of the self-complementary nucleic acid normally occupied by a mutant ITR. In some embodiments, sequence encoding a hairpin-forming RNA forms a shRNA, miRNA, or AmiRNA.

In some embodiments, an AmiRNA construct comprises: a nucleic acid sequence encoding a pri-miRNA scaffold; a nucleic acid sequence encoding a guide strand; and, a nucleic acid sequence encoding a passenger strand, wherein, the pri-miRNA scaffold is derived from a naturally-occurring pri-miRNA and comprises at least one flanking sequence and a loop-forming sequence comprising at least 4 nucleotides.

In some embodiments, the guide strand of an AmiRNA and the passenger strand of an AmiRNA share at least 50% complementarity to a target nucleic acid sequence but are not 100% complementary to one another. In some embodiments, the nucleic acid sequence encoding the guide strand and the nucleic acid sequence encoding the passenger strand are inserted into the pri-miRNA scaffold between the flanking sequence and the loop-forming sequence, thereby forming a stem.

In some embodiments, the nucleic acid sequence encoding the guide strand of an AmiRNA and the nucleic acid sequence encoding the passenger strand of an AmiRNA have at least one base pair mismatch. In some embodiments, the nucleic acid sequence encoding the guide strand and the nucleic acid sequence encoding the passenger strand have two base pair mismatches, three base pair mismatches, four base pair mismatches, five base pair mismatches, six base pair mismatches, seven base pair mismatches, eight base pair mismatches, nine base pair mismatches, ten base pair mismatches, eleven base pair mismatches, twelve base pair mismatches, thirteen base pair mismatches, fourteen base pair mismatches or fifteen base pair mismatches. In some embodiments, the nucleic acid sequence encoding the guide strand and the nucleic acid sequence encoding the passenger strand have mismatches at no more than ten consecutive base pairs. In some embodiments, at least one base pair mismatch is located at an anchor position. In some embodiments, at least one base pair mismatch is located in a center portion of the stem.

In some embodiments, the pri-miRNA scaffold is derived from a pri-miRNA selected from the group consisting of pri-MIR-21, pri-MIR-22, pri-MIR-26a, pri-MIR-30a, pri-MIR-33, pri-MIR-122, pri-MIR-375, pri-MIR-199, pri-MIR-99, pri-MIR-194, pri-MIR-155, and pri-MIR-451.

In some embodiments, the guide strand of an AmiRNA targets a gene associated with a gain of function mutation disease, an oncogene, or a gene associated with a metabolic disorder.

In some embodiments, the guide strand of an AmiRNA targets SOD1, Huntington gene, p53, HER2/neu, LDLR, or beta-glucosidase.

In some embodiments, the size of a single stranded nucleic acid is in a range of 300 bp to 10 kb.

In some embodiments, ITRs of rAAV vectors described herein are AAV1, AAV2, AAV3, AAV4, AAV5, or AAV6 ITRs.

In some aspects, the disclosure provides an rAAV vector comprising an artificial miRNA (AmiRNA) construct.

In some aspects, the disclosure provides a preparation comprising a plurality of rAAVs, wherein at least 80% of the rAAVs comprise a non-truncated genome having a sequence encoding an artificial miRNA (AmiRNA).

In some embodiments, a non-truncated genome comprises two ITRs flanking the sequence encoding an artificial miRNA (AmiRNA). In some embodiments, at least 90% of the rAAVs comprise a non-truncated genome having a sequence encoding an artificial miRNA (AmiRNA). In some embodiments, at least 95% of the rAAVs comprise a non-truncated genome having a sequence encoding an artificial miRNA (AmiRNA). In some embodiments, at least 99% of the rAAVs comprise a non-truncated genome having a sequence encoding an artificial miRNA (AmiRNA).

In some aspects, the disclosure provides a self-complementary adeno-associated virus (scAAV) comprising: a viral genome comprising a nucleic acid sequence encoding at least one inverted terminal repeat and a promoter operably linked with a nucleic acid sequence encoding a hairpin-forming RNA; and at least one AAV capsid protein serotype.

In some embodiments, the nucleic acid sequence encoding a hairpin-forming RNA is between two inverted terminal repeats.

In some embodiments, the size of a scAAV viral genome is between about 150 bp and 5 kb.

In some embodiments, the disclosure relates to a host cell comprising an rAAV vector, nucleic acid encoding an rAAV vector, or a scAAV as described by the disclosure.

In some aspects, the disclosure provides a kit comprising a container housing an rAAV vector, nucleic acid encoding an rAAV vector, or a scAAV as described by the disclosure. In some embodiments, the container is a syringe.

### BRIEF DESCRIPTION OF DRAWINGS

FIGS. 1A-1B show the yield of scAAV vectors embedded with or without shRNA cassettes. FIG. 1A, depicts the structure of a scAAV vector carrying shRNA cassette next to wild-type ITR. FIG. 1B shows the AAV yield analyzed by quantitative-PCR.

FIGS. 2A-2E show the effects of the shRNA cassette position within scAAV plasmids on RNAi efficacy, reporter gene expression and AAV production. FIG. 2A depicts the scAAV plasmids harboring the shRNA cassette near the mutated ITR, in the intron, and near the wild-type ITR. FIG. 2B shows the levels of Firefly luciferase (Fluc) and Relina luciferase activity 48 hours after equal amounts of scAAV-shFluc plasmids were co-transfected with psiCheck-2 plasmid into 293HEK cells. FIG. 2C shows EGFP expression of scAAV vectors. FIG. 2D shows vector yield of scAAV plasmids harboring shRNA against Fluc or Apob at different positions that were packaged into AAV9, as determined by qPCR. FIG. 2E shows the comparison of RNAi efficacy from scAAV plasmids carrying shApob at different locations. After 48 hours, EGFP expression was observed and the cell lysis was used for firefly luciferase and beta-gal activity assay. The pmiCheck-Apob plasmid was constructed by incorporating partial Apob cDNA after β-Galactosidase gene in pmiCheck plasmid. Fluc reporter gene was served as control for transfection efficacy. The ratio between β-Galactosidase and Fluc activity reflects the shApob activity in cells. Values are mean±s.d.

FIGS. 3A-3D show the analysis of truncated AAV genomes in viral vector DNA and Hirt's DNA from 293HEK cells after triple transfection. FIG. 3A shows SYBR gold staining of full length and truncated viral genomes from scAAV9-shFluc vectors. FIG. 3B shows southern blot analysis of Hirt's DNA from 293HEK cells after triple-transfection with scAAV9-shFluc vectors for 48 or 72 hours was probed by an EGFP fragment. FIG. 3C shows SYBR gold staining of full length and truncated viral genomes from a scAAV9-shApob vectors. FIG. 3D shows southern blot analysis of Hirt's DNA from 293HEK cells after triple-transfection with scAAV9-shApob for 48 or 72 hours was probed by an EGFP fragment.

FIGS. 4A-4C show an examination of the AAV viral genomes from AAV8, AAV9, AAVrh 10, and AAV2 carrying shRNA or artificial miRNA cassettes against different genes in the intron region (FIG. 4A); AAV9 carrying different shRNA sequence proximal or distal to wild-type ITR (FIG. 4B); and AAV6, AAV8, and AAV9 harboring shRNA or artificial miRNA distal to mutant ITR (FIG. 4C). Vector DNA equivalent to 0.1−1×E11 GC viral genomes was loaded in 1% agarose gel and stained with SYBR Gold.H1/U6, H1, or U6 promoter.

FIGS. 5A-5C show the impacts of shFluc cassettes on single-stranded AAV vector genome truncation and production. FIG. 5A depicts the locations of shFluc in the ssAAV genome. FIG. 5B shows the viral genome DNA. FIG. 5C shows vector yield from ssAAV-shFluc.

FIGS. 6A-6D show that short hairpin DNA compromises the scAAV genome integrity. FIG. 6A shows a model for a shRNA sequence on AAV genome replication. G. 6B shows DNA extracted from AAV vectors was examined on alkaline agarose gel. FIG. 6C shows restriction enzyme digestion of genome of AAV vectors carrying shApob cassettes in the intron. DNA isolated from AAV vectors was probed with an EGFP fragment with or without Msc I digestion.

FIGS. 7A-7C show shRNA-encoding DNA functions as a mutant ITR in AAV genome replication and vector production. FIG. 7A depicts constructs used in the study. shApob or shFluc cassettes were integrated into the intron or upstream of CB promoter in the absence of mutant ITR. scAAV plasmids without mITR or Wt-ITR were used as controls. SEQ ID NO:2 is scAAV-CBEGFP; SEQ ID NO: 1 is Intron-D; SEQ ID NO: 7 is NoshRNA; SEQ ID NO: 8 is pshRNA+wtTR−; SEQ ID NO: 4 is pH1-shApob1.3; SEQ ID NO: 3 is pH1-shApob1.5; SEQ ID NO: 6 is pH1-shApob2.2; SEQ ID NO: 5 is pH1-shApob2.0; and SEQ ID NO: 9 is pU6-shFluc1.3. FIG. 7B shows a Southern blot analysis of Hirt's DNA from 293HEK cells transfected with the constructs in FIG. 7A, adeno-helper plasmid, and Rep2/Cap9 plasmid for 48 hours. The EGFP fragments were labelled by P32 as probe using a random labelling kit from Takara. FIG. 7C depicts viral genome DNA from AAV vectors containing WT FR and hairpin DNA at two ends.

FIGS. 8A-8F show the thermodynamic stability of the DNA encoding shRNAs determine the truncation of AAV genome. FIG. 8A depicts the rational design of shApob. The guide strand of shApob remains unchanged and singular or multiple bulges were introduced into different positions. The sequences, from top to bottom, correspond to SEQ ID NOs: 35-54. FIG. 8B shows a Southern blot analysis of Hirt's DNA from 293HEK cells co-transfected with a scAAV-shApob plasmid, pAdeno-helper plasmid, and pRep2/Cap9 plasmid. The intensity of the truncated and full-length genomes was measured using Image J. FIG. 8C shows the correlation between the portion of the AAV truncated genome and the short hairpin DNA thermodynamic stability. The dG was calculated by RNAfold. FIG. 8D illustrates the ratio of Gal and Fluc in 293EK cells co-transfected with shApob constructs and a pmiCHECK-Apob sensor plasmid. FIG. 8E presents the small RNA Northern blot analysis of pre-shApob and antisense-Apob in 293HEK cells transfected with the indicated shApob constructs. FIG. 8F shows the Apob silencing efficacy of shApob contains certain bulges at a lower ratio of shApob plasmids and the sensor plasmid.

FIGS. 9A-9G show the development of AAV-compatible gene silencing construct using pri-miRNA scaffold. FIG. 9A depicts the viral genome of scAAV8 vectors carrying the pri-miRNA fragment. The pri-miRNA fragment was amplified by PCR from the C57/B6 mouse genome DNA, including the pre-rniRNA flanked with about 100 bps up- and down-stream nucleotides and integrated into the intron between the Gluc reporter gene and CB promoter in the scAAV plasmid. The constructs were packaged into AAV8 vectors and viral genome DNA was run on a 1% agarose gel. FIG. 9B shows the design of AAV-compatible gene silencing constructs. The guide strand of the miRNA was replaced with the shApob guide strand, and the passenger strand and flanking sequence were changed based on the structure of the pre-miRNA in the design of AAV-compatible gene silencing constructs. The sequences, from top to bottom, correspond to SEQ ID NOs: 55-58. FIG. 9C illustrates gene silencing constructs that were co-transfected with pmiCHECK-Apob sensor plasmid at a 1:3 ratio into 293HEK and Huh7.5 cells. After 48 hours, Fluc and Gal levels were measured and the ratio between Gal and Fluc was calculated. FIG. 9D shows the ratio of Gal and Fluc levels in 293HEK cells co-transfected pri-miR-451, pri-miR-26a, and pri-miR-33 scaffolds with pmiCHECK-Apob plasmid at the ratio of 1:3, 1:1, and 1:0.33. FIG. 9E depicts a Northern blot analysis of Apob antisense small RNA in 293HEK cells transfected with shRNA or miRNA scaffold constructs. U6 RNA was used as a loading control. FIGS. 9F and 9G show that miRNA scaffolds improve the integrity of the scAAV genome. scAAV plasmids carrying shApob or miApob scaffolds were transfected with pAdeno-helper and Rep2/Cap9 plasmids into 293HEK cells. Southern blot analysis was performed on the Hirt's DNA after 48 hours of triple-transfection using a Gluc probe (FIG. 9F). FIG. 9G shows the agarose gel of viral genome extracted from the AAV preps.

FIGS. 10A-10B present comparisons of reporter gene expression and target gene silencing efficacy between shApob and miR-33 Apob in mice. FIG. 10A shows the Gaussia luciferase expression in mouse serum from mice that received IV-delivered AAV9 carrying shApob or miR-33 Apob at the indicated doses. FIG. 10B shows the relative quantification of Apob rnRNA in mouse livers by gRT-PCR.

FIGS. 11A-11F show in vivo performance of scAAV-shApob vectors and analysis of the truncated AAV molecules. 1×10¹² GCs scAAV9-shApob was administrated to 6-8 week old C57/B6 mice through tail vein. After 3 weeks, scrum ALT was measured (FIG. 11A), relative Apob expression was analyzed by qRT-PCR (FIG. 11B), EGFP expression in liver was observed (FIG. 11C). Six mice were used in each group. (FIG. 11D) Southern blot analysis of AAV molecular forms using EGFP probe in liver. The liver DNA was digested with EcoR I or Msc I before hybridization. There is one Msc I site in the wtTR region and no EcoR I site in the vector genome. (FIG. 11E) Amplification of the junction connected to wtITR by Inverted PCR. (FIG. 11F) Sequence of TOPO colonies from PCR products by inverted PCR. The sequences, from top to bottom, correspond to SEQ ID NOs: 59-71. Values are mean±s.d.

FIG. 12 shows a ssAAV construct incorporating shFluc cassette at different locations co-transfected with pAd and pRep/Cap into HEK293 cells. After 48 hours of transfection, Hirt DNA was extracted and probed with the GFP or Neo probes, respectively. The black solid circles indicate the shFluc locations. L0.2 represents the shRNA is 0.2 kb away to the L-TR. R0.2 represents the shRNA is 0.2 kb away to the R-TR.

FIGS. 13A-13C show the characterization of the truncated AAV genomes. FIG. 13A shows the strategy for the preparation of library for SMRT sequencing and data process. Model guided AAV sequence prediction (FIG. 13B) and sequence of scAAV and truncated AAV genomes (FIG. 13C) are also shown. RBE, Rep binding element. B-B′ and C-C′ are two palindromes in TR. A, replicated A in vector genome.

FIGS. 14A-14I show the production of AAV vectors flanked one wtTR and one hairpin DNA at two ends and the functionality evaluation in mice. FIG. 14A shows pCis constructs used for AAV production. SEQ ID NO: 10 is U6-shFluc1.3. FIG. 14B shows prediction of packaged genome size based on the hairpin DNA position. FIG. 14C shows Southern blot analysis of the Hirt DNA from triple-transfection using EGFP probe. FIG. 14D shows viral genome DNA from purified vectors in native agarose gel and alkaline gel. FIG. 14E shows EGFP expression in the liver of mice received 3×10¹¹ GCs of AAV vectors from tail vein for 3 weeks. FIG. 14F shows Southern blot analysis of the EcoR I or Msc I digested liver DNA using EGFP probe. FIG. 14G shows qRT-PCR analysis of Apob mRNA and small RNA Northern blot analysis in mouse liver. FIG. 14H shows alkaline gel analysis of H1-shApob1.3 and H1-shApob1.5 shAAV genomes. FIG. 14I shows an illustration of the production of shRNA from AAV vectors. Values are mean±s.d. Four mice were used in each group.

FIGS. 15A-15D show hairpin DNA function as mutant TR in AAV package and in vivo transduction. FIG. 15A shows a prediction of the secondary structure from CB promoter sequence by RNAfold. FIG. 15B shows AAV yield of scAAV9 and shAAV9 vectors. The titers were determined by qPCR. FIG. 15C shows re-engineering of wtTR in scAAV genome (SEQ ID NO: 72). In the reservation of RBE, A, trs and D elements, RBE-D-A element was created by replacing the B-B′ and C-C′ with a shRNA loop (TTCAAGAGA), T-Apob and T-PCI were made by replacing the B-B′ and C-C′ with non-relevant sequence which can maintain the T-shape structure. The Cis plasmids with modified wtTR were co-transfected with pAd and pRcp/Cap plasmids into HEK293 cells for 48 hours. Hirt DNA was extracted and probed with EGFP fragment. SEQ ID NO: 12 is shApob1.3-(RBE-A-D); SEQ ID NO: 17 is shFluc 1.3-(RBE-A-D); SEQ ID NO: 15 is shApob2.0-(RBE-A-D); SEQ ID NO: 13 is shApob1.3-TApob; SEQ ID NO: 18 is shFluc1.3-TApob; SEQ ID NO: 16 is shApob2.0-TApob; SEQ ID NO: 14 is shApob1.3-TApob; SEQ ID NO: 19 is shFluc1.3-TPC1; and SEQ ID NO: 11 is shApob2.0-TPC1. FIG. 15D shows SMRT sequence analysis of H1-Apob1.3 and H1-Apob1.5 shAAV vector genomes.

FIGS. 16A-16C show positioning of shRNA cassettes within scAAV constructs impacts vector yield. FIG. 16A shows yield comparison of independent scAAV8 preparations with (n=15) or without (n=11) shRNA cassettes designed proximal to the wtTR. FIG. 16B shows a schematic of scAAV plasmids consisting of a CMV enhancer/Chicken β-actin promoter (CB), an EGFP reporter gene, and a beta-globin polyA sequence (PA). shRNA cassettes against Apob, driven by the H1 promoter; or the Firefly luciferase gene (Fluc), driven by the U6 promoter was inserted adjacent to the mTR (m-P and m-D), within the intron (Intron-P and Intron-D), or adjacent to the wtTR (Wt-D and Wt-P). FIG. 16C shows vectors depicted in FIG. 16B were packaged into AAV9 capsids and assessed for yield by quantitating genome copy number (GC) using an EGFP primer/probe set.

FIGS. 17A-17E show in vivo performances of scAAV-shApob vectors and analysis of small AAV molecules. FIG. 17A shows qPCR analysis of hepatic Apob expression 3 weeks after injection of PBS or scAAV9-shApob vectors (5×10¹³ GCs/kg) into 6- to 8-week old C57/B6 mice. Expression levels are represented as relative apob mRNA levels normalized to actin levels. FIG. 17B shows EGFP expression in livers as determined by fluorescence microscopy. Bar=100 μM. FIG. 17C shows Southern blot analysis of AAV molecular forms in livers by probing against EGFP sequence. Liver DNAs were digested with EcoRI (non-cutter), or MscI (single cutter within the wtTR) prior to hybridization FIG. 17D shows a diagram showing the detection of wtTR junctions in circular AAV molecules by inverse PCR. Intron-Rev and PA-For primers are designed in opposing directions to span only circularized DNA templates. Total DNA from the livers of mice receiving AAV-shApob vectors was used as template. FIG. 17E shows TOPO sequences of the inverse PCR products from mice that received Intron-P and Intron-D vectors using total liver DNA as template. The shRNA cassette depicted here comprises an H1 promoter and an shRNA sequence, which consists of a passenger strand, and a guide strand, connected by a loop sequence. The sequences, from top to bottom, correspond to SEQ ID NOs: 59-71. Values are mean±s.d. Six mice were used in each group.

FIGS. 18A-18E show profiling of truncated genomes produced by AAV vectors containing shRNA cassettes. FIG. 18A shows agarose gel analysis of scAAV vector genomes carrying shApob, driven by the H1 promoter; or shFluc, driven by the U6 promoter at different positions. FIG. 18B shows AAV vector genomes (AAV8, AAV9, AAVrh10, and AAV2) carrying intronic shRNA cassettes against different genes. FIG. 18C shows AAV9 genomes carrying different shRNA sequence inserted between the EGFP transgene and the wtTR. FIG. 18D shows AAV6 and AAV8 genomes harboring shRNA cassettes inserted between the mTR and the CB promoter. Vector DNA equivalents of 0.1−1×10¹¹ GC viral genomes was loaded on 1% agarose gels and stained with SYBR Gold. sh-1 to sh-26 represents 26 different shDNA sequences. FIG. 18E shows the molar ratio of truncated genomes to full-length genomes in AAV vectors carrying shDNA at different positions. Ratios were calculated by normalizing their band intensities by densitometry to their molecular sizes. The ratio of truncated to full-length genomes of Wt-P (n=5), Wt-D (n=5), Intron-D (n=12), Intron-P (n=2), m-D (n=9), and m-P (n=2) preparations are reported on a log scale. Values are mean±s.d.

FIGS. 19A-19C show truncated genomes in Hirt DNA from 293 cells transfected with scAAV or ssAAV constructs. FIG. 19A shoes scAAV Constructs carrying shApob or shFluc were co-transfected with pAd helper plasmid and pRep2/Cap9 or pRep2/Cap8 plasmid into 293 cells. After 48 or 72 hours, Hirt DNA was extracted and probed with EGFP fragment. FIG. 19B shows a schematic of ssAAV constructs carrying shFluc cassette at different locations. The black solid circles indicate the shFluc locations. FIG. 19C shows Southern blot analysis of the Hirt DNA samples from 293 cells co-transfected with pAd helper plasmid, pRep2/Cap9 plasmid and pCis plasmids (Indicated in FIG. 19B) for 48 hours with GFP or Neo probe. Unlike scAAV, the replication of ssAAV genomes can start from either left or right TR.

FIGS. 20A-20F show characterization of truncated AAV genomes. FIG. 20A shows a model of conventional scAAV genome replication. AAV genome replication initiates from the wtTR and generates intra-molecular double-stranded genomes. FIG. 20B shows a model of AAV genome replication detoured by a short DNA hairpin. FIG. 20C shows DNAs extracted from AAV vectors were examined on an alkaline agarose gel. FIG. 19D shows a schematic diagram showing the strategy of library preparation for SMRT sequencing and data processing. FIG. 20E shows model-guided sequence prediction of truncated AAV genomes. Functional segments of the mTR are displayed: Rep binding element (RBE), the B-B′ hairpin, and the C-C′ hairpin. “A”, represents the replicated A domain in the vector genome. FIG. 20F shows SMRT sequencing reads aligned to custom references that represent self-complementary sequence resulting from template-switching events at the mTR (top panel), and the shApob-encoding sequences (middle panel, Intron-D; and bottom panel, Intron-P).

FIGS. 21A-21C show restriction enzyme digestion of the AAV genomes carrying shApob cassette in the intron (Intron-P and Intron-D). FIG. 21A shows the location of restriction enzymes (RE) in the Intron-P and Intron-D vectors. Three restriction enzymes (MluI, XhoI and BstXI) that recognize the sites located upstream of shDNA were chosen to excise only the full-length AAV genomes, while three other restriction enzymes (EagI, HindIII and MscI) that recognize the sites located downstream of shDNA were selected to digest both full-length and truncated genomes. FIG. 21B shows restriction enzyme mapping on the vector genome. MluI, XhoI and BstXI that recognize the upstream of the shApob encoding sequence only digest the full-length genome. EagI, HindIII and MscI that recognize the downstream digest both full-length and truncated genome. FIG. 21C shows agarose gel analysis of vector genome of the Intron-P and Intron-D vectors after digestion by the REs as indicated.

FIGS. 22A-22K show characterization of shAAV genome and in vivo evaluation of shAAV vectors. FIG. 22A shows a schematic of pCis constructs used for AAV production. FIG. 22B shows Southern blot analysis of the Hirt DNA from transfected HEK293 cells using an EGFP probe. FIG. 22C shows viral genome DNA from purified of vectors (˜1.0×10¹⁰ GCs) in native (left panel) and alkaline (right panel) agarose gels. FIG. 22D shows EGFP expression in the livers of adult mice i.v. treated with AAV (1.6×10¹³ GCs/kg) for 3 weeks. FIG. 22E shows Southern blot analysis of the EcoRI or MscI digested liver DNA using an EGFP probe. FIG. 22F shows qRT-PCR analysis of Apob mRNA and small RNA Northern blot analysis of mouse livers. Mice were administrated with AAV vectors (1.6×10¹³ GCs/kg) for three weeks. FIG. 22G shows alkaline agarose gel analysis of H1-shApob1.3 and H1-shApob1.5 shAAV genomes. DNA extracted from AAV vectors (˜1.5×10¹⁰ GCs) were digested with PstI, BglII, or BstBI, separated on a 0.8% alkaline agarose gel, and stained with SYBR Gold. FIG. 22H shows a diagram of replication products from the H1-shApob1.3 shAAV vector, illustrating re-direction at the shRNA expression cassette to produce 1.3-kb species, or read through products. The percentages of read-through genomes and shAAV genomes were calculated by their band intensities by densitometry, normalized to their molecular sizes. FIG. 22I shows a schematic of pCis constructs lacking PolIII promoters. FIG. 22J shows EGFP expression and FIG. 22K shows qPCR analysis of Apob mRNA and Northern blot analysis of Apob antisense small RNA from mouse liver at 3 weeks post injection with 1.6×10¹³ GCs/kg shAAV9 vectors that packaged constructs from FIG. 22I. Bar=100 μm. Values are mean±s.d. Four mice were used in each group.

FIGS. 23A-23B show sequence analysis of H1-Apob1.3 (FIG. 23A) and H1-Apob1.5 (FIG. 23B) shAAV vector genomes. The intra-molecular double-stranded genomes and the missing sequences were indicated for both shAAV genomes.

FIG. 24 shows comparisons of the gene silencing efficacy of scAAV9 vectors carrying shApob-encoding sequence in different positions. Six to eight weeks old C57/B6 mice were intravenously injected with scAAV9-shApob vectors at the indicating doses. The mice were sacrificed three weeks later and expression of Apob gene and transduced AAV genome copies in liver were analyzed by qRT-PCR and qPCR, respectively. Three mice were used in each group treated with 1×10¹², 2×10¹¹ and 4×10¹⁰ GCs per mouse. Five mice were used in each group treated with 5×10⁹ GCs/mouse. Values are mean±s.d.

FIGS. 25A-25B show SMRT sequencing reads of whole-vector genomes of the Intron-D construct, or the scAAVOCB6-PI-EGFP construct mapped to their respective references, related to FIGS. 26A-26C. Reads in fastq format where halved to map only the sense strand of self-complementary molecules. Reads mapped by BWA-MEM were visualized with IGV to display only a subset of genomes to illustrate the full distribution of genome heterogeneity. Alignments were thus downsized to display a single representative read per sequence length. IGV display is set to show the base pair compositions of reads.

FIGS. 26A-26C show characterization of variable vector genomes generated from shDNA-like sequences. FIG. 26A shows aggregation plots of alignment termination positions along the pH1-shAPob1.3 construct (top panel), or the scAAV-EGFP construct (bottom panel) as assessed by direct SMRT sequencing of AAV genomes. Positional tags were distributed into intervals of 10 nt bins and the density of tags were plotted along the H1-shApob1.3 vector sequence. Peaks indicate regions along the genome where termination hotspots occur. Sequences of discovered hotspots are flanked by inverted repeats (IR). The linear sequences in the CMV enhancer (IR-1 and IR-2), CB promoter (IR-3), and the EGFP reporter gene (IR-4) are displayed below. FIG. 26B shows the secondary structures of IR1-4 using RNA Fold. Sequences in grey highlight the inverted repeat sequences. Underlined sequences reside outside of the inverted repeat region. The sequences are as follows: IR-1 (SEQ ID NO: 73), IR-2 (SEQ ID NO: 74), IR-3 (SEQ ID NO: 75), and IR-4 (SEQ ID NO: 76). FIG. 26C shows sequence alignments of AAV genomes to a reference consisting of self-complementary strands flanking the IR-3 sequence (Top). The bottom panel details the loop sequence that connects the partial CB-promoter and its reverse complementary sequence. The sequence corresponds to SEQ ID NO: 77.

FIG. 27 shows self-complementary genomes with IR 1, IR 2 and IR 4 loops. Alignments were made with reference genomes that contain complementary sequences at two sides and IR 1, IR 2 or IR 4 in the middle to the SMRT reads. The complementary sequences span from the wtTR and the IR 1, IR 2, and IR 4, respectively. The alignment was done in SMRT reads from both shAAV and scAAV vectors. The sequences, from top to bottom, correspond to SEQ ID NOs: 78-80.

FIG. 28 shows gene silencing by pri-miRNA scaffolds. The gene silencing driven by H1 (top) or CB (bottom) promoters was assessed using omiCHECK-Apob in HEK-293 cells. Fluc and Gal levels were measured and ratio between Gal and Fluc was calculated. Agarose gel electrophoresis of viral genomes was performed (right).

### DETAILED DESCRIPTION OF INVENTION

Adeno-associated virus (AAV) is a small (˜26 nm) replication-defective, non-enveloped virus, that generally depends on the presence of a second virus, such as adenovirus or herpes virus, for its growth in cells. AAV is not known to cause disease and induces a very mild immune response. AAV can infect both dividing and non-dividing cells and may incorporate its genome into that of the host cell. These features make AAV a very attractive candidate for creating viral vectors for gene therapy. Modified AAV-based vectors, referred to as recombinant AAV (rAAV) vectors, generally comprise two AAV inverted terminal repeat (ITR) sequences separated by a transgene. Transgenes capable of being delivered by rAAV vectors include, but are not limited to, nucleic acids encoding peptides and polypeptides, and RNAi molecules (e.g., dsRNA, siRNA, shRNA, miRNA, AmiRNA, etc.). However, the introduction of nucleic acid sequences encoding hairpin-forming RNA (e.g., shRNA, miRNA, and AmiRNA) has deleterious effects on rAAV genome replication and rAAV yield. Accordingly, new rAAV vectors that allow efficient replication and generate improved rAAV yield are needed.

In some aspects, the instant disclosure provides rAAV (e.g., self-complementary AAV; scAAV) vectors comprising a single-stranded self-complementary nucleic acid with inverted terminal repeats (ITRs) at each of two ends and a central portion comprising a promoter operably linked with a sequence encoding a hairpin-forming RNA. In some embodiments, the sequence encoding a hairpin-forming RNA is substituted at a position of the self-complementary nucleic acid normally occupied by a mutant ITR. In some embodiments, the disclosure provides an isolated nucleic acid having one inverted terminal repeat at a first terminus and a promoter operably linked with a sequence encoding a hairpin-forming RNA at a second terminus, wherein the isolated nucleic acid forms a self-complementary AAV (scAAV) vector.

### Self-Complementary AAV (scAAV) Vectors

As used herein, the term “self-complementary AAV vector” (scAAV) refers to a vector containing a double-stranded vector genome generated by the absence of a terminal resolution site (TR) from one of the ITRs of the AAV. The absence of a TR prevents the initiation of replication at the vector terminus where the TR is not present. In general, scAAV vectors generate single-stranded, inverted repeat genomes, with a wild-type (wt) AAV TR at each end and a mutated TR (mTR) in the middle. The instant invention is based, in part, on the recognition that DNA fragments encoding RNA hairpin structures (e.g., shRNA, miRNA, and AmiRNA) can serve a function similar to a mutant inverted terminal repeat (mITR) during viral genome replication, generating self-complementary AAV vector genomes. For example, in some embodiments, the disclosure provides rAAV (e.g., self-complementary AAV; scAAV) vectors comprising a single-stranded self-complementary nucleic acid with inverted terminal repeats (ITRs) at each of two ends and a central portion comprising a promoter operably linked with a sequence encoding a hairpin-forming RNA. In some embodiments, the sequence encoding a hairpin-forming RNA is substituted at a position of the self-complementary nucleic acid normally occupied by a mutant ITR.

### Recombinant AAV vectors

In some aspects, the disclosure provides an rAAV vector comprising a single-stranded self-complementary nucleic acid with inverted terminal repeats (ITRs) at each of two ends and a central portion comprising a promoter operably linked with a sequence encoding a hairpin-forming RNA.

“Recombinant AAV (rAAV) vectors” are typically composed of, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The instant disclosure provides a vector comprising a single, cis-acting wild-type ITR. In some embodiments, the ITR is a 5′ ITR. In some embodiments, the ITR is a 3′ ITR Generally, ITR sequences are about 145 bp in length. Preferably, substantially the entire sequences encoding the ITR(s) is used in the molecule, although some degree of minor modification of these sequences is permissible. The ability to modify ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). For example, an ITR may be mutated at its terminal resolution site (TR), which inhibits replication at the vector terminus where the TR has been mutated and results in the formation of a self-complementary AAV. Another example of such a molecule employed in the present disclosure is a “cis-acting” plasmid containing the transgene, in which the selected transgene sequence and associated regulatory elements are flanked by the 5′ AAV ITR sequence and a 3′ hairpin-forming RNA sequence. AAV ITR sequences may be obtained from any known AAV, including presently identified mammalian AAV types. In some embodiments, an ITR sequence is an AAV2, AAV3, AAV4, AAV5, AAV6, AAV7, AAV8, AAVrh8, AAV9, AAV10, and/or AAVrh10 ITR sequence.

In some embodiments, the rAAVs of the disclosure are pseudotyped rAAVs. For example, a pseudotyped AAV vector containing the ITRs of serotype X encapsidated with the proteins of Y will be designated as AAVX/Y (e.g., AAV2/1 has the ITRs of AAV2 and the capsid of AAV1). In some embodiments, pseudotyped rAAVs may be useful for combining the tissue-specific targeting capabilities of a capsid protein from one AAV serotype with the viral DNA from another AAV serotype, thereby allowing targeted delivery of a transgene to a target tissue.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the disclosure. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

As used herein, a nucleic acid sequence (e.g., coding sequence) and regulatory sequences are said to be “operably” linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., shRNA, miRNA, miRNA inhibitor).

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al, and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931; and Klump, H et al., Gene Therapy, 2001; 8: 811-817).

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors of the disclosure may optionally include 5′ leader or signal sequences. The choice and design of an appropriate vector is within the ability and discretion of one of ordinary skill in the art.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ccdysone insect promoter (No et al, Proc. Natl. Acad. Sci. USA, 93:3346-3351 (1996)), the tetracycline-repressible system (Gossen et al, Proc. Natl. Acad. Sci. USA, 89:5547-5551 (1992)), the tetracycline-inducible system (Gossen et al, Science, 268:1766-1769 (1995), sec also Harvey et al, Curr. Opin. Chem. Biol., 2:512-518 (1998)), the RU486-inducible system (Wang et al, Nat. Biotech., 15:239-243 (1997) and Wang et al, Gene Ther., 4:432-441 (1997)) and the rapamycin-inducible system (Magari et al, J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene (e.g., hairpin forming nucleic acid) will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor a-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some aspects, the disclosure relates to a host cell comprising an rAAV vector. Generally, host cells are useful for amplifying and/or packaging rAAV vectors. The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. In some embodiments, a host cell is a 293 cell, HeLa cell, A549 cell, or a SF9 cell. Still other stable host cells may be generated by one of skill in the art.

The recombinant AAV vector, rep sequences, cap sequences, and helper functions required for producing the rAAV of the disclosure may be delivered to the packaging host cell using any appropriate genetic element (vector). In some embodiments, a single nucleic acid encoding all three capsid proteins (e.g., VP1, VP2 and VP3) is delivered into the packaging host cell in a single vector. In some embodiments, nucleic acids encoding the capsid proteins are delivered into the packaging host cell by two vectors; a first vector comprising a first nucleic acid encoding two capsid proteins (e.g., VP1 and VP2) and a second vector comprising a second nucleic acid encoding a single capsid protein (e.g., VP3). In some embodiments, three vectors, each comprising a nucleic acid encoding a different capsid protein, are delivered to the packaging host cell. The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. Sec, e.g., K. Fisher et al, J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

### Isolated Nucleic Acids

In some aspects, the disclosure relates to an isolated nucleic acid having one inverted terminal repeat at a first terminus and a promoter operably linked with a sequence encoding a hairpin-forming RNA at a second terminus, wherein the isolated nucleic acid forms a self-complementary AAV (scAAV) vector. In some embodiments, the sequence encoding a hairpin-forming RNA is substituted at a position of the scAAV vector normally occupied by a mutant ITR.

A “nucleic acid” sequence refers to a DNA or RNA sequence. In some embodiments, proteins and nucleic acids of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The skilled artisan will also realize that conservative amino acid substitutions may be made to provide functionally equivalent variants, or homologs of the capsid proteins. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (c) S, T; (f) Q, N; and (g) E, D. Therefore, one can make conservative amino acid substitutions to the amino acid sequence of the proteins and polypeptides disclosed herein. Furthermore, nucleic acids can be tailored for optimal gene expression based on optimization of nucleotide sequence to reflect the codon bias of a host cell. The skilled artisan appreciates that gene expression may be improved if codon usage is biased towards those codons favored by the host.

A “self-complementary nucleic acid” refers to a nucleic acid capable of hybridizing with itself (i.e., folding back upon itself) to form a single-stranded duplex structure, due to the complementarity (e.g., base-pairing) of the nucleotides within the nucleic acid strand. Self-complementary nucleic acids can form a variety of secondary structures, such as hairpin loops, loops, bulges, junctions and internal bulges. Certain self-complementary nucleic acids (e.g., miRNA, shRNA, AmiRNA) perform regulatory functions, such as gene silencing. Self-complementary nucleic acids having AAV ITRs can form self-complementary AAVs.

The degree of complementarity between the nucleotide bases of a self-complementary nucleic acid affects the stability (e.g., thermodynamic stability) of the molecule's secondary structure. For example, mismatches present in the duplex region of the self-complementary nucleic acid can form additional bulges or loops, thereby lowering the thermodynamic stability of the structure formed by the nucleic acid. In some aspects, the instant disclosure is based, in part, on the recognition that lowering the thermodynamic stability of a hairpin-forming self-complementary nucleic acid allows the nucleic acid to function as a mutant ITR in a self-complementary AAV vector. In some embodiments, the thermostability of a self-complementary nucleic acid is lowered by mutating the nucleic acid to introduce at least 1, at least 2, at least 3, at least 4, at least 5, at least 6, at least 7 at least 8, at least 9, or at least 10 mismatches in the duplex forming region. In some embodiments, the nucleic acid is mutated to introduce more than 10 mismatches in the duplex region. Mismatches can also be introduced into the non-duplex-forming region of the nucleic acid.

### Transgenes

The composition of the transgene sequence of the rAAV vector will depend upon the use to which the resulting vector will be put. For example, one type of transgene sequence includes a reporter sequence, which upon expression produces a detectable signal. In another example, the transgene encodes a therapeutic protein or therapeutic functional RNA. In another example, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. Appropriate transgene coding sequences will be apparent to the skilled artisan.

The disclosure is based, in part, on the discovery that transgenes comprising hairpin-forming nucleic acids with decreased thermostability are useful for replacing mutant ITRs in self-complementary AAV vectors. In some embodiments, nucleic acids described herein increase scAAV vector replication and packaging efficiency. In some aspects, the disclosure relates to rAAVs and rAAV vectors comprising a transgene, wherein the transgene is a hairpin-forming RNA. Non-limiting examples of hairpin-forming RNA include short hairpin RNA (shRNA), microRNA (miRNA) and artificial microRNA (AmiRNA). In some embodiments, nucleic acids are provided herein that contain or encode the target recognition and binding sequences (e.g., a seed sequence or a sequence complementary to a target) of any one of the inhibitory RNAs (e.g., shRNA, miRNA, AmiRNA) disclosed herein.

Generally, hairpin-forming RNAs are arranged into a self-complementary “stem-loop” structure that includes a single nucleic acid encoding a stem portion having a duplex comprising a sense strand (e.g., passenger strand) connected to an antisense strand (e.g., guide strand) by a loop sequence. The passenger strand and the guide strand share complementarity. In some embodiments, the passenger strand and guide strand share 100% complementarity. In some embodiments, the passenger strand and guide strand share at least 50%, at least 60%, at least 70%, at least 80%, at least 90%, at least 95%, or at least 99% complementarity. A passenger strand and a guide strand may lack complementarity due to a base-pair mismatch. In some embodiments, the passenger strand and guide strand of a hairpin-forming RNA have at least 1, at least 2, at least 3, at least 4, at least 5, at least 6, at least 7 at least 8, at least 9, or at least 10 mismatches. Generally, the first 2-8 nucleotides of the stem (relative to the loop) are referred to as “seed” residues and play an important role in target recognition and binding. The first residue of the stem (relative to the loop) is referred to as the “anchor” residue. In some embodiments, hairpin-forming RNA have a mismatch at the anchor residue.

Hairpin-forming RNA are useful for translational repression and/or gene silencing via the RNAi pathway. Due to having a common secondary structure, hairpin-forming RNA share the characteristic of being processed by the proteins Drosha and Dicer prior to being loaded into the RNA-induced silencing complex (RISC). Duplex length amongst hairpin-forming RNA can vary. In some embodiments, a duplex is between about 19 nucleotides and about 200 nucleotides in length. In some embodiments, a duplex is between about between about 14 nucleotides to about 35 nucleotides in length. In some embodiments, a duplex is between about 19 and 150 nucleotides in length. In some embodiments, hairpin-forming RNA has a duplex region that is 19, 20, 21, 22, 23, 24, 25, 26, 27, 28, 29, 30, 31, 32, or 33 nucleotides in length. In some embodiments, a duplex is between about 19 nucleotides and 33 nucleotides in length. In some embodiments, a duplex is between about 40 nucleotides and 100 nucleotides in length. In some embodiments, a duplex is between about 60 and about 80 nucleotides in length.

In some embodiments, the hairpin-forming RNA is a microRNA (miRNA), or artificial microRNA (AmiRNA). A microRNA (miRNA) is a small non-coding RNA found in plants and animals and functions in transcriptional and post-translational regulation of gene expression. An artificial microRNA (AmiRNA) is derived by modifying native miRNA to replace natural targeting regions of pre-mRNA with a targeting region of interest. For example, a naturally occurring, expressed miRNA can be used as a scaffold or backbone (e.g., a pri-miRNA scaffold), with the stem sequence replaced by that of an miRNA targeting a gene of interest. An artificial precursor microRNA (pre-amiRNA) is normally processed such that one single stable small RNA is preferentially generated. In some embodiments, scAAV vectors and scAAVs described herein comprise a nucleic acid encoding an AmiRNA. In some embodiments, the pri-miRNA scaffold of the AmiRNA is derived from a pri-miRNA selected from the group consisting of pri-MIR-21, pri-MIR-22, pri-MIR-26a, pri-MIR-30a, pri-MIR-33, pri-MIR-122, pri-MIR-375, pri-MIR-199, pri-MIR-99, pri-MIR-194, pri-MIR-155, and pri-MIR-451.

The following non-limiting list of miRNA genes, and their homologues, which are also useful in certain embodiments of the vectors provided herein: hsa-let-7a, hsa-let-7a*, hsa-let-7b, hsa-let-7b*, hsa-let-7c, hsa-let-7c*, hsa-let-7d, hsa-let-7d*, hsa-let-7c, hsa-let-7c*, hsa-let-7f, hsa-let-7f-1*, hsa-let-7f-2*, hsa-let-7g, hsa-let-7g*, hsa-let-7i, hsa-let-7i*, hsa-miR-1, hsa-miR-100, hsa-miR-100*, hsa-miR-101, hsa-miR-101*, hsa-miR-103, hsa-miR-105, hsa-miR-105*, hsa-miR-106a, hsa-miR-106a*, hsa-miR-106b, hsa-miR-106b*, hsa-miR-107, hsa-miR-10a, hsa-miR-10a*, hsa-miR-10b, hsa-miR-10b*, hsa-miR-1178, hsa-miR-1179, hsa-miR-1180, hsa-miR-1181, hsa-miR-1182, hsa-miR-1183, hsa-miR-1184, hsa-miR-1185, hsa-miR-1197, hsa-miR-1200, hsa-miR-1201, hsa-miR-1202, hsa-miR-1203, hsa-miR-1204, hsa-miR-1205, hsa-miR-1206, hsa-miR-1207-3p, hsa-miR-1207-5p, hsa-miR-1208, hsa-miR-122, hsa-miR-122*, hsa-miR-1224-3p, hsa-miR-1224-5p, hsa-miR-1225-3p, hsa-miR-1225-5p, hsa-miR-1226, hsa-miR-1226*, hsa-miR-1227, hsa-miR-1228, hsa-miR-1228*, hsa-miR-1229, hsa-miR-1231, hsa-miR-1233, hsa-miR-1234, hsa-miR-1236, hsa-miR-1237, hsa-miR-1238, hsa-miR-124, hsa-miR-124*, hsa-miR-1243, hsa-miR-1244, hsa-miR-1245, hsa-miR-1246, hsa-miR-1247, hsa-miR-1248, hsa-miR-1249, hsa-miR-1250, hsa-miR-1251, hsa-miR-1252, hsa-miR-1253, hsa-miR-1254, hsa-miR-1255a, hsa-miR-1255b, hsa-miR-1256, hsa-miR-1257, hsa-miR-1258, hsa-miR-1259, hsa-miR-125a-3p, hsa-miR-125a-5p, hsa-miR-125b, hsa-miR-125b-1*, hsa-miR-125b-2*, hsa-miR-126, hsa-miR-126*, hsa-miR-1260, hsa-miR-1261, hsa-miR-1262, hsa-miR-1263, hsa-miR-1264, hsa-miR-1265, hsa-miR-1266, hsa-miR-1267, hsa-miR-1268, hsa-miR-1269, hsa-miR-1270, hsa-miR-1271, hsa-miR-1272, hsa-miR-1273, hsa-miR-127-3p, hsa-miR-1274a, hsa-miR-1274b, hsa-miR-1275, hsa-miR-127-5p, hsa-miR-1276, hsa-miR-1277, hsa-miR-1278, hsa-miR-1279, hsa-miR-128, hsa-miR-1280, hsa-miR-1281, hsa-miR-1282, hsa-miR-1283, hsa-miR-1284, hsa-miR-1285, hsa-miR-1286, hsa-miR-1287, hsa-miR-1288, hsa-miR-1289, hsa-miR-129*, hsa-miR-1290, hsa-miR-1291, hsa-miR-1292, hsa-miR-1293, hsa-miR-129-3p, hsa-miR-1294, hsa-miR-1295, hsa-miR-129-5p, hsa-miR-1296, hsa-miR-1297, hsa-miR-1298, hsa-miR-1299, hsa-miR-1300, hsa-miR-1301, hsa-miR-1302, hsa-miR-1303, hsa-miR-1304, hsa-miR-1305, hsa-miR-1306, hsa-miR-1307, hsa-miR-1308, hsa-miR-130a, hsa-miR-130a*, hsa-miR-130b, hsa-miR-130b*, hsa-miR-132, hsa-miR-132*, hsa-miR-1321, hsa-miR-1322, hsa-miR-1323, hsa-miR-1324, hsa-miR-133a, hsa-miR-133b, hsa-miR-134, hsa-miR-135a, hsa-miR-135a*, hsa-miR-135b, hsa-miR-135b*, hsa-miR-136, hsa-miR-136*, hsa-miR-137, hsa-miR-138, hsa-miR-138-1*, hsa-miR-138-2*, hsa-miR-139-3p, hsa-miR-139-5p, hsa-miR-140-3p, hsa-miR-140-5p, hsa-miR-141, hsa-miR-141*, hsa-miR-142-3p, hsa-miR-142-5p, hsa-miR-143, hsa-miR-143*, hsa-miR-144, hsa-miR-144*, hsa-miR-145, hsa-miR-145*, hsa-miR-146a, hsa-miR-146a*, hsa-miR-146b-3p, hsa-miR-146b-5p, hsa-miR-147, hsa-miR-147b, hsa-miR-148a, hsa-miR-148a*, hsa-miR-148b, hsa-miR-148b*, hsa-miR-149, hsa-miR-149*, hsa-miR-150, hsa-miR-150*, hsa-miR-151-3p, hsa-miR-151-5p, hsa-miR-152, hsa-miR-153, hsa-miR-154, hsa-miR-154*, hsa-miR-155, hsa-miR-155*, hsa-miR-15a, hsa-miR-15a*, hsa-miR-15b, hsa-miR-15b*, hsa-miR-16, hsa-miR-16-1*, hsa-miR-16-2*, hsa-miR-17, hsa-miR-17*, hsa-miR-181a, hsa-miR-18la*, hsa-miR-181a-2*, hsa-miR-181b, hsa-miR-181c, hsa-miR-181c*, hsa-miR-181d, hsa-miR-182, hsa-miR-182*, hsa-miR-1825, hsa-miR-1826, hsa-miR-1827, hsa-miR-183, hsa-miR-183*, hsa-miR-184, hsa-miR-185, hsa-miR-185*, hsa-miR-186, hsa-miR-186*, hsa-miR-187, hsa-miR-187*, hsa-miR-188-3p, hsa-miR-188-5p, hsa-miR-18a, hsa-miR-18a*, hsa-miR-18b, hsa-miR-18b*, hsa-miR-190, hsa-miR-190b, hsa-miR-191, hsa-miR-191*, hsa-miR-192, hsa-miR-192*, hsa-miR-193a-3p, hsa-miR-193a-5p, hsa-miR-193b, hsa-miR-193b*, hsa-miR-194, hsa-miR-194*, hsa-miR-195, hsa-miR-195*, hsa-miR-196a, hsa-miR-196a*, hsa-miR-196b, hsa-miR-197, hsa-miR-198, hsa-miR-199a-3p, hsa-miR-199a-5p, hsa-miR-199b-5p, hsa-miR-19a, hsa-miR-19a*, hsa-miR-19b, hsa-miR-19b-1*, hsa-miR-19b-2*, hsa-miR-200a, hsa-miR-200a*, hsa-miR-200b, hsa-miR-200b*, hsa-miR-200c, hsa-miR-200c*, hsa-miR-202, hsa-miR-202*, hsa-miR-203, hsa-miR-204, hsa-miR-205, hsa-miR-206, hsa-miR-208a, hsa-miR-208b, hsa-miR-20a, hsa-miR-20a*, hsa-miR-20b, hsa-miR-20b*, hsa-miR-21, hsa-miR-21*, hsa-miR-210, hsa-miR-211, hsa-miR-212, hsa-miR-214, hsa-miR-214*, hsa-miR-215, hsa-miR-216a, hsa-miR-216b, hsa-miR-217, hsa-miR-218, hsa-miR-218-1*, hsa-miR-218-2*, hsa-miR-219-1-3p, hsa-miR-219-2-3p, hsa-miR-219-5p, hsa-miR-22, hsa-miR-22*, hsa-miR-220a, hsa-miR-220b, hsa-miR-220c, hsa-miR-221, hsa-miR-221*, hsa-miR-222, hsa-miR-222*, hsa-miR-223, hsa-miR-223*, hsa-miR-224, hsa-miR-23a, hsa-miR-23a*, hsa-miR-23b, hsa-miR-23b*, hsa-miR-24, hsa-miR-24-1*, hsa-miR-24-2*, hsa-miR-25, hsa-miR-25*, hsa-miR-26a, hsa-miR-26a-1*, hsa-miR-26a-2*, hsa-miR-26b, hsa-miR-26b*, hsa-miR-27a, hsa-miR-27a*, hsa-miR-27b, hsa-miR-27b*, hsa-miR-28-3p, hsa-miR-28-5p, hsa-miR-296-3p, hsa-miR-296-5p, hsa-miR-297, hsa-miR-298, hsa-miR-299-3p, hsa-miR-299-5p, hsa-miR-29a, hsa-miR-29a*, hsa-miR-29b, hsa-miR-29b-1*, hsa-miR-29b-2*, hsa-miR-29c, hsa-miR-29c*, hsa-miR-300, hsa-miR-301a, hsa-miR-301b, hsa-miR-302a, hsa-miR-302a*, hsa-miR-302b, hsa-miR-302b*, hsa-miR-302c, hsa-miR-302c*, hsa-miR-302d, hsa-miR-302d*, hsa-miR-302c, hsa-miR-302f, hsa-miR-30a, hsa-miR-30a*, hsa-miR-30b, hsa-miR-30b*, hsa-miR-30c, hsa-miR-30c-1*, hsa-miR-30c-2*, hsa-miR-30d, hsa-miR-30d*, hsa-miR-30c, hsa-miR-30c*, hsa-miR-31, hsa-miR-31*, hsa-miR-32, hsa-miR-32*, hsa-miR-320a, hsa-miR-320b, hsa-miR-320c, hsa-miR-320d, hsa-miR-323-3p, hsa-miR-323-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335, hsa-miR-335*, hsa-miR-337-3p, hsa-miR-337-5p, hsa-miR-338-3p, hsa-miR-338-5p, hsa-miR-339-3p, hsa-miR-339-5p, hsa-miR-33a, hsa-miR-33a*, hsa-miR-33b, hsa-miR-33b*, hsa-miR-340, hsa-miR-340*, hsa-miR-342-3p, hsa-miR-342-5p, hsa-miR-345, hsa-miR-346, hsa-miR-34a, hsa-miR-34a*, hsa-miR-34b, hsa-miR-34b*, hsa-miR-34c-3p, hsa-miR-34c-5p, hsa-miR-361-3p, hsa-miR-361-5p, hsa-miR-362-3p, hsa-miR-362-5p, hsa-miR-363, hsa-miR-363*, hsa-miR-365, hsa-miR-367, hsa-miR-367*, hsa-miR-369-3p, hsa-miR-369-5p, hsa-miR-370, hsa-miR-371-3p, hsa-miR-371-5p, hsa-miR-372, hsa-miR-373, hsa-miR-373*, hsa-miR-374a, hsa-miR-374a*, hsa-miR-374b, hsa-miR-374b*, hsa-miR-375, hsa-miR-376a, hsa-miR-376a*, hsa-miR-376b, hsa-miR-376c, hsa-miR-377, hsa-miR-377*, hsa-miR-378, hsa-miR-378*, hsa-miR-379, hsa-miR-379*, hsa-miR-380, hsa-miR-380*, hsa-miR-381, hsa-miR-382, hsa-miR-383, hsa-miR-384, hsa-miR-409-3p, hsa-miR-409-5p, hsa-miR-410, hsa-miR-411, hsa-miR-411*, hsa-miR-412, hsa-miR-421, hsa-miR-422a, hsa-miR-423-3p, hsa-miR-423-5p, hsa-miR-424, hsa-miR-424*, hsa-miR-425, hsa-miR-425*, hsa-miR-429, hsa-miR-431, hsa-miR-431*, hsa-miR-432, hsa-miR-432*, hsa-miR-433, hsa-miR-448, hsa-miR-449a, hsa-miR-449b, hsa-miR-450a, hsa-miR-450b-3p, hsa-miR-450b-5p, hsa-miR-451, hsa-miR-452, hsa-miR-452*, hsa-miR-453, hsa-miR-454, hsa-miR-454*, hsa-miR-455-3p, hsa-miR-455-5p, hsa-miR-483-3p, hsa-miR-483-5p, hsa-miR-484, hsa-miR-485-3p, hsa-miR-485-5p, hsa-miR-486-3p, hsa-miR-486-5p, hsa-miR-487a, hsa-miR-487b, hsa-miR-488, hsa-miR-488*, hsa-miR-489, hsa-miR-490-3p, hsa-miR-490-5p, hsa-miR-491-3p, hsa-miR-491-5p, hsa-miR-492, hsa-miR-493, hsa-miR-493*, hsa-miR-494, hsa-miR-495, hsa-miR-496, hsa-miR-497, hsa-miR-497*, hsa-miR-498, hsa-miR-499-3p, hsa-miR-499-5p, hsa-miR-500, hsa-miR-500*, hsa-miR-501-3p, hsa-miR-501-5p, hsa-miR-502-3p, hsa-miR-502-5p, hsa-miR-503, hsa-miR-504, hsa-miR-505, hsa-miR-505*, hsa-miR-506, hsa-miR-507, hsa-miR-508-3p, hsa-miR-508-5p, hsa-miR-509-3-5p, hsa-miR-509-3p, hsa-miR-509-5p, hsa-miR-510, hsa-miR-511, hsa-miR-512-3p, hsa-miR-512-5p, hsa-miR-513a-3p, hsa-miR-513a-5p, hsa-miR-513b, hsa-miR-513c, hsa-miR-514, hsa-miR-515-3p, hsa-miR-515-5p, hsa-miR-516a-3p, hsa-miR-516a-5p, hsa-miR-516b, hsa-miR-517*, hsa-miR-517a, hsa-miR-517b, hsa-miR-517c, hsa-miR-518a-3p, hsa-miR-518a-5p, hsa-miR-518b, hsa-miR-518c, hsa-miR-518c*, hsa-miR-518d-3p, hsa-miR-518d-5p, hsa-miR-518c, hsa-miR-518c*, hsa-miR-518f, hsa-miR-518f*, hsa-miR-519a, hsa-miR-519b-3p, hsa-miR-519c-3p, hsa-miR-519d, hsa-miR-519c, hsa-miR-519c*, hsa-miR-520a-3p, hsa-miR-520a-5p, hsa-miR-520b, hsa-miR-520c-3p, hsa-miR-520d-3p, hsa-miR-520d-5p, hsa-miR-520c, hsa-miR-520f, hsa-miR-520g, hsa-miR-520h, hsa-miR-521, hsa-miR-522, hsa-miR-523, hsa-miR-524-3p, hsa-miR-524-5p, hsa-miR-525-3p, hsa-miR-525-5p, hsa-miR-526b, hsa-miR-526b*, hsa-miR-532-3p, hsa-miR-532-5p, hsa-miR-539, hsa-miR-541, hsa-miR-541*, hsa-miR-542-3p, hsa-miR-542-5p, hsa-miR-543, hsa-miR-544, hsa-miR-545, hsa-miR-545*, hsa-miR-548a-3p, hsa-miR-548a-5p, hsa-miR-548b-3p, hsa-miR-548b-5p, hsa-miR-548c-3p, hsa-miR-548c-5p, hsa-miR-548d-3p, hsa-miR-548d-5p, hsa-miR-548c, hsa-miR-548f, hsa-miR-548g, hsa-miR-548h, hsa-miR-548i, hsa-miR-548j, hsa-miR-548k, hsa-miR-5481, hsa-miR-548m, hsa-miR-548n, hsa-miR-5480, hsa-miR-548p, hsa-miR-549, hsa-miR-550, hsa-miR-550*, hsa-miR-551a, hsa-miR-551b, hsa-miR-551b*, hsa-miR-552, hsa-miR-553, hsa-miR-554, hsa-miR-555, hsa-miR-556-3p, hsa-miR-556-5p, hsa-miR-557, hsa-miR-558, hsa-miR-559, hsa-miR-561, hsa-miR-562, hsa-miR-563, hsa-miR-564, hsa-miR-566, hsa-miR-567, hsa-miR-568, hsa-miR-569, hsa-miR-570, hsa-miR-571, hsa-miR-572, hsa-miR-573, hsa-miR-574-3p, hsa-miR-574-5p, hsa-miR-575, hsa-miR-576-3p, hsa-miR-576-5p, hsa-miR-577, hsa-miR-578, hsa-miR-579, hsa-miR-580, hsa-miR-581, hsa-miR-582-3p, hsa-miR-582-5p, hsa-miR-583, hsa-miR-584, hsa-miR-585, hsa-miR-586, hsa-miR-587, hsa-miR-588, hsa-miR-589, hsa-miR-589*, hsa-miR-590-3p, hsa-miR-590-5p, hsa-miR-591, hsa-miR-592, hsa-miR-593, hsa-miR-593*, hsa-miR-595, hsa-miR-596, hsa-miR-597, hsa-miR-598, hsa-miR-599, hsa-miR-600, hsa-miR-601, hsa-miR-602, hsa-miR-603, hsa-miR-604, hsa-miR-605, hsa-miR-606, hsa-miR-607, hsa-miR-608, hsa-miR-609, hsa-miR-610, hsa-miR-611, hsa-miR-612, hsa-miR-613, hsa-miR-614, hsa-miR-615-3p, hsa-miR-615-5p, hsa-miR-616, hsa-miR-616*, hsa-miR-617, hsa-miR-618, hsa-miR-619, hsa-miR-620, hsa-miR-621, hsa-miR-622, hsa-miR-623, hsa-miR-624, hsa-miR-624*, hsa-miR-625, hsa-miR-625*, hsa-miR-626, hsa-miR-627, hsa-miR-628-3p, hsa-miR-628-5p, hsa-miR-629, hsa-miR-629*, hsa-miR-630, hsa-miR-631, hsa-miR-632, hsa-miR-633, hsa-miR-634, hsa-miR-635, hsa-miR-636, hsa-miR-637, hsa-miR-638, hsa-miR-639, hsa-miR-640, hsa-miR-641, hsa-miR-642, hsa-miR-643, hsa-miR-644, hsa-miR-645, hsa-miR-646, hsa-miR-647, hsa-miR-648, hsa-miR-649, hsa-miR-650, hsa-miR-651, hsa-miR-652, hsa-miR-653, hsa-miR-654-3p, hsa-miR-654-5p, hsa-miR-655, hsa-miR-656, hsa-miR-657, hsa-miR-658, hsa-miR-659, hsa-miR-660, hsa-miR-661, hsa-miR-662, hsa-miR-663, hsa-miR-663b, hsa-miR-664, hsa-miR-664*, hsa-miR-665, hsa-miR-668, hsa-miR-671-3p, hsa-miR-671-5p, hsa-miR-675, hsa-miR-7, hsa-miR-708, hsa-miR-708*, hsa-miR-7-1*, hsa-miR-7-2*, hsa-miR-720, hsa-miR-744, hsa-miR-744*, hsa-miR-758, hsa-miR-760, hsa-miR-765, hsa-miR-766, hsa-miR-767-3p, hsa-miR-767-5p, hsa-miR-768-3p, hsa-miR-768-5p, hsa-miR-769-3p, hsa-miR-769-5p, hsa-miR-770-5p, hsa-miR-802, hsa-miR-873, hsa-miR-874, hsa-miR-875-3p, hsa-miR-875-5p, hsa-miR-876-3p, hsa-miR-876-5p, hsa-miR-877, hsa-miR-877*, hsa-miR-885-3p, hsa-miR-885-5p, hsa-miR-886-3p, hsa-miR-886-5p, hsa-miR-887, hsa-miR-888, hsa-miR-888*, hsa-miR-889, hsa-miR-890, hsa-miR-891a, hsa-miR-891b, hsa-miR-892a, hsa-miR-892b, hsa-miR-9, hsa-miR-9*, hsa-miR-920, hsa-miR-921, hsa-miR-922, hsa-miR-923, hsa-miR-924, hsa-miR-92a, hsa-miR-92a-1*, hsa-miR-92a-2*, hsa-miR-92b, hsa-miR-92b*, hsa-miR-93, hsa-miR-93*, hsa-miR-933, hsa-miR-934, hsa-miR-935, hsa-miR-936, hsa-miR-937, hsa-miR-938, hsa-miR-939, hsa-miR-940, hsa-miR-941, hsa-miR-942, hsa-miR-943, hsa-miR-944, hsa-miR-95, hsa-miR-96, hsa-miR-96*, hsa-miR-98, hsa-miR-99a, hsa-miR-99a*, hsa-miR-99b, and hsa-miR-99b*. In some embodiments, the above miRNAs may be encoded for in a vector provided herein (e.g., in a hairpin nucleic acid that replaces a mutant ITR). In some embodiments, sequences of the foregoing miRNAs may be useful as scaffolds or as targeting regions (e.g., seed regions of AmiRNA).

A miRNA inhibits the function of the mRNAs it targets and, as a result, inhibits expression of the polypeptides encoded by the mRNAs. Thus, blocking (partially or totally) the activity of the miRNA (e.g., silencing the miRNA) can effectively induce, or restore, expression of a polypeptide whose expression is inhibited (derepress the polypeptide). In one embodiment, derepression of polypeptides encoded by mRNA targets of a miRNA is accomplished by inhibiting the miRNA activity in cells through any one of a variety of methods. For example, blocking the activity of a miRNA can be accomplished by hybridization with a small interfering nucleic acid (e.g., antisense oligonucleotide, miRNA sponge, TuD RNA) that is complementary, or substantially complementary to, the miRNA, thereby blocking interaction of the miRNA with its target mRNA. As used herein, an small interfering nucleic acid that is substantially complementary to a miRNA is one that is capable of hybridizing with a miRNA, and blocking the miRNA's activity. In some embodiments, an small interfering nucleic acid that is substantially complementary to a miRNA is an small interfering nucleic acid that is complementary with the miRNA at all but 1, 2, 3, 4, 5, 6, 7, 8, 9, 10, 11, 12, 13, 14, 15, 16, 17, or 18 bases. In some embodiments, an small interfering nucleic acid sequence that is substantially complementary to a miRNA, is an small interfering nucleic acid sequence that is complementary with the miRNA at, at least, one base.

In some embodiments, the rAAV vectors described herein further comprise a protein-encoding transgene. In some embodiments, the protein coding gene located upstream of the hairpin forming nucleic acid of the rAAV vector. For example, rAAV vectors described herein can further comprise a therapeutic protein or a reporter protein. Reporter sequences that may be provided in a transgene include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer. Such reporters can, for example, be useful in verifying the tissue-specific targeting capabilities and tissue specific promoter regulatory activity of an rAAV.

In some embodiments, the rAAV vectors described herein further comprise a therapeutic protein. Such rAAV may be useful for preventing or treating one or more genetic deficiencies or dysfunctions in a mammal, such as for example, a polypeptide deficiency or polypeptide excess in a mammal, and particularly for treating or reducing the severity or extent of deficiency in a human manifesting one or more of the disorders linked to a deficiency in such polypeptides in cells and tissues. Exemplary therapeutic proteins include one or more polypeptides selected from the group consisting of growth factors, interleukins, interferons, anti-apoptosis factors, cytokines, anti-diabetic factors, anti-apoptosis agents, coagulation factors, anti-tumor factors. Other non-limiting examples of therapeutic proteins include BDNF, CNTF, CSF, EGF, FGF, G-SCF, GM-CSF, gonadotropin, IFN, IFG-1, M-CSF, NGF, PDGF, PEDF, TGF, VEGF, TGF-B2, TNF, prolactin, somatotropin, XIAP1, IL-1, IL-2, IL-3, IL-4, IL-5, IL-6, IL-7, IL-8, IL-9, IL-10, IL-10 (187A), viral IL-10, IL-11, IL-12, IL-13, IL-14, IL-15, IL-16 IL-17, and IL-18.

In some aspects, the disclosure relates to rAAV comprising a combination of hairpin-forming nucleic acid and a protein coding gene. rAAV vectors comprising an interfering nucleic acid and a protein coding gene are useful for simultaneously performing gene silencing and gene substitution. For example, rAAV vectors described herein can be used to silence a defective gene (e.g., mutated SOD1) while simultaneously delivering a non-mutated or functional copy of the defective gene (e.g., wild-type SOD1).

Certain transgenes may exceed the cloning capacity of traditional rAAV vectors (e.g., transgenes larger than about 4.8 kb). However, methods for the delivery of large therapeutic proteins by rAAV vectors, for example as disclosed by Lai et al., Nat Biotechnol., 23(11): 1435-1439, 2005; Flotte, Respir. Res., 1: 16-18, 2000; Duan et al., Nat. Med., 6(5): 595-598, 2000; Sun et al., Nat. Med., 6(5): 599-602; each of which references is incorporated herein by reference in its entirety, have been developed. These methods rely on the capability of rAAV vectors to undergo genome concatenation and trans-splicing in host cells. For example, fragments of a large gene (e.g., >4.8 kb) may be encoded on several rAAV vectors and delivered to a host cell. Upon entry into the host cell, the rAAV vector genomes concatenate and trans-splice the fragments of the transgene, resulting in reconstitution of the full-length transgene. Therefore, in some embodiments, the disclosure relates to a composition comprising a plurality of rAAV vectors, wherein each rAAV vector of the plurality encodes a fragment of a transgene such that introduction of the composition to a host cell will result in the production of the full-length transgene encoded by the fragments.

In some embodiments, rAAV vectors comprise a transgene to be transferred to a subject to treat a disease associated with reduced expression, lack of expression or dysfunction of the gene. Exemplary genes and associated disease states include, but are not limited to: glucose-6-phosphatase, associated with glycogen storage deficiency type 1A; phosphoenolpyruvate-carboxykinase, associated with Pepck deficiency; galactose-1 phosphate uridyl transferase, associated with galactosemia; phenylalanine hydroxylase, associated with phenylketonuria; branched chain alpha-ketoacid dehydrogenase, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; omithine transcarbamylase, associated with omithine transcarbamylase deficiency; argininosuccinic acid synthetase, associated with citrullinemia; low density lipoprotein receptor protein, associated with familial hypercholesterolemia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotinidase, associated with biotinidase deficiency; beta-glucocerebrosidase, associated with Gaucher discase; beta-glucuronidase, associated with Sly syndrome; peroxisome membrane protein 70 kDa, associated with Zellweger syndrome; porphobilinogen deaminase, associated with acute intermittent porphyria; alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic discases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin for the treatment of diabetes.

In some embodiments, the disclosure relates to an AAV comprising a nucleic acid encoding a protein or functional RNA useful for the treatment of a condition, disease or disorder associated with the central nervous system (CNS). The following is a non-limiting list of genes associated with CNS disease: DRD2, GRIA1, GRIA2, GRIN1, SLC1A1, SYP, SYT1, CHRNA7, 3Rtau/4rTUS, APP, BAX, BCL-2, GRIK1, GFAP, IL-1, AGER, associated with Alzheimer's Disease; UCH-L1, SKP1, EGLN1, Nurr-1, BDNF, TrkB, gstm1, S106β, associated with Parkinson's Disease; IT15, PRNP, JPH3, TBP, ATXN1, ATXN2, ATXN3, Atrophin 1, FTL, TITF-1, associated with Huntington's Disease; FXN, associated with Freidrich's ataxia; ASPA, associated with Canavan's Disease; DMD, associated with muscular dystrophy; and SMN1, UBE1, DYNC1H1 associated with spinal muscular atrophy. In some embodiments, the disclosure relates to recombinant AAVs comprising nucleic acids that express one or more of the foregoing genes or fragments thereof. In some embodiments, the disclosure relates to recombinant AAVs comprising nucleic acids that express one or more functional RNAs that inhibit expression of one or more of the foregoing genes.

In some embodiments, rAAV vectors described by the disclosure comprise AmiRNA having a guide strand that targets genes related to diseases caused by gain of function mutations. Generally, gain of function mutations confer new or enhanced activity on a protein. Examples of genes in which a gain of function mutation causes disease include SOD1 (Amyotrophic lateral sclerosis, ALS), huntington (Huntington's disease, HD) and beta globulin (sickle cell disease). In some embodiments, rAAV vectors described by the disclosure comprise AmiRNA having a guide strand that targets one or more oncogenes. Oncogenes are gene that has the potential to cause cancer, and are often mutated or expressed at high levels. Examples of oncogenes include p53, HER2/neu, and c-Myc. In some embodiments, rAAV vectors described by the disclosure comprise AmiRNA having a guide strand that targets genes involved in metabolic pathways (e.g., lipogenesis). Dysfunction of metabolic genes is associated with several diseases, including Gaucher disease (beta-glucosidase), Tay-Sachs disease (beta-hexosaminidase A), and familial hypercholesterolemia (low-density lipoprotein receptor, LDLR).

The skilled artisan will also realize that in the case of transgenes encoding proteins or polypeptides, that mutations that results in conservative amino acid substitutions may be made in a transgene to provide functionally equivalent variants, or homologs of a protein or polypeptide. In some aspects the disclosure embraces sequence alterations that result in conservative amino acid substitution of a transgene. In some embodiments, the transgene comprises a gene having a dominant negative mutation. For example, a transgene may express a mutant protein that interacts with the same elements as a wild-type protein, and thereby blocks some aspect of the function of the wild-type protein.

### Recombinant AAV Administration Methods

The rAAVs may be delivered to a subject in compositions according to any appropriate methods known in the art. The rAAV, preferably suspended in a physiologically compatible carrier (e.g., in a composition), may be administered to a subject, e.g., host animal, such as a human, mouse, rat, cat, dog, sheep, rabbit, horse, cow, goat, pig, guinea pig, hamster, chicken, turkey, or a non-human primate (e.g, Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000).

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some embodiments the rAAV is administered at a dose of 10¹⁰, 10¹¹, 10¹², 10¹³, 10¹⁴, or 10¹⁵ genome copies per subject. In some embodiments the rAAV is administered at a dose of 10¹⁰, 10¹¹, 10¹², 10¹³, or 10¹⁴ genome copies per kg. In some cases, a dosage between about 10¹¹ to 10¹² rAAV genome copies is appropriate. In certain embodiments, 10¹² rAAV genome copies is effective to target heart, liver, and pancreas tissues. In some cases, stable transgenic animals are produced by multiple doses of an rAAV.

In some embodiments, rAAV compositions are formulated to reduce aggregation of AAV particles in the composition, particularly where high rAAV concentrations are present (e.g., ˜10¹³ GC/ml or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intraopancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimcrosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 .ANG., containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (e.g., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### Kits and Related Compositions

The agents described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to an animal, such as a syringe, topical application devices, or iv needle tubing and bag, particularly in the case of the kits for producing specific somatic animal models.

The kit may have a variety of forms, such as a blister pouch, a shrink wrapped pouch, a vacuum scalable pouch, a scalable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The instructions included within the kit may involve methods for constructing an AAV vector as described herein. In addition, kits of the disclosure may include, instructions, a negative and/or positive control, containers, diluents and buffers for the sample, sample preparation tubes and a printed or electronic table of reference AAV sequence for sequence comparisons.

### EXAMPLES

### Example 1: Novel rAAV Genome Designs using Artificial Hairpin Loop Structures to Replace at Least One AAV Inverted Terminal Repeat (ITR)

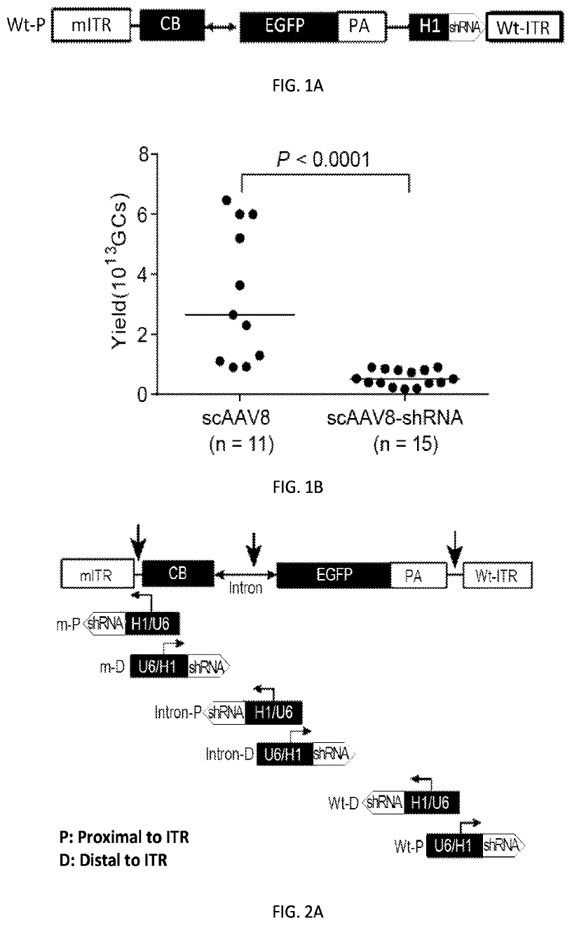

When scAAV vectors carrying shRNA cassettes are produced next to wild type ITRs in the genome, the yield is much lower than scAAV vectors without shRNA cassettes (FIGS. 1A and 1B). In the production process, the vector genome flanked with two ITRs is excised from the rAAV vector plasmid (FIG. 1A), replicated, and packaged into AAV capsids. scAAV genome replication can only start from the wild-type ITR (Wt-ITR) due to the mutation in the other ITR (mITR). The tight hairpin structure of shRNA-encoding DNA next to the Wt-ITR inhibits AAV genome replication and leads to the poor vector yield.

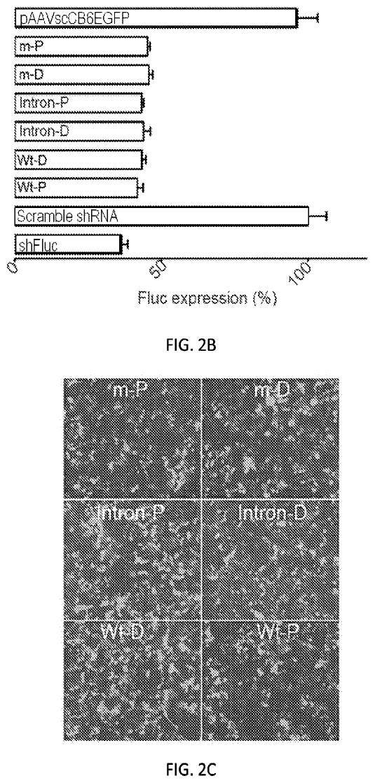

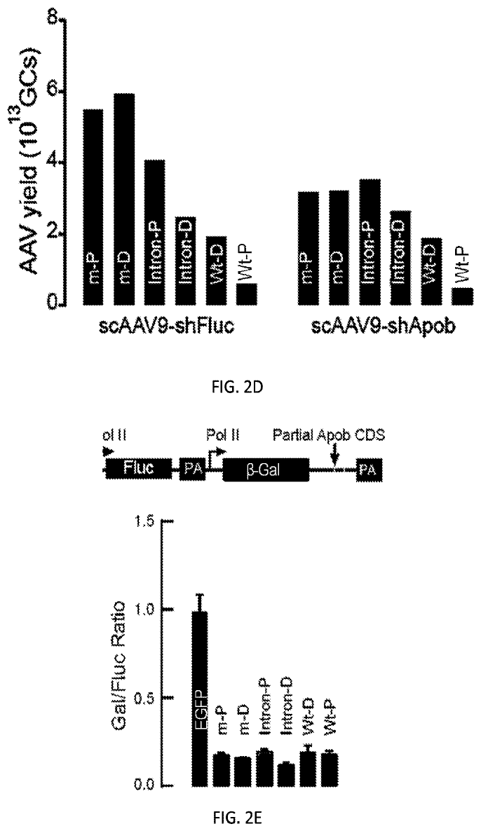

The location of the shRNA cassette in the AAV genome was changed to avoid the positioning effect on genome replication. Two shRNA cassettes, H1-shApob and U6-shFluc, expressing shRNAs that target endogenous the mouse Apob gene and firefly luciferase transgene, respectively, were used to test positional effects. The shRNA cassettes were cloned into different locations in the scAAV vector plasmid as shown in FIG. 2A. Relocated shRNA cassettes in the vector genome did not affect the RNAi efficacy or control transgene EGFP expression in 293HEK cells (FIGS. 2B and 2C), but did improve the vector yield 5-10 fold (FIG. 2D).

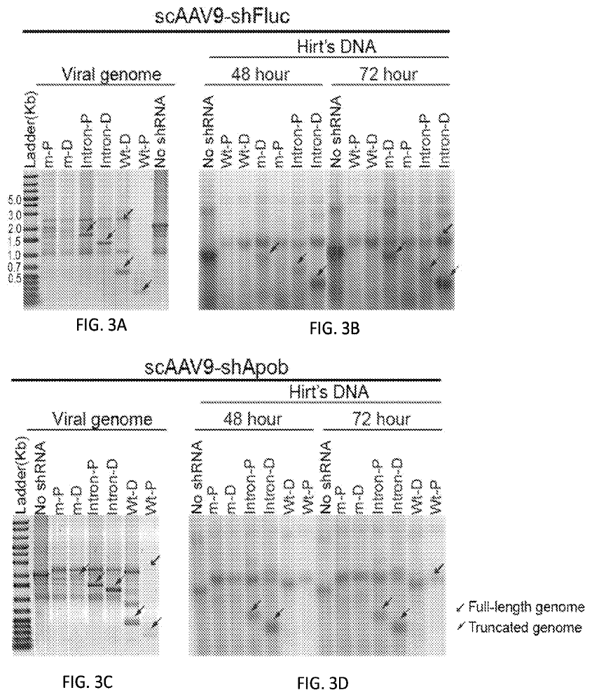

In the genome DNA extracted from the purified viral vector preparations, in addition to the expected full-length genome, truncated vector genomes were found to be packaged in sizes that correlated with the distance from the Wt-ITR to the location of the shRNA cassettes in the vector genome (FIGS. 3A and 3C). Non-genomic Hirt's DNAs prepared from triple transfected 293 cells in a small scale rAAV production experiment were analyzed by Southern blot using an EGFP probe (FIGS. 3B and 3D). Consistent with the AAV vector genome designs (FIGS. 2A and 2B), the truncated AAV molecules were found in the AAV genome replication stage (FIGS. 3B and 3D), indicating the shRNA-encoding DNA is a barrier to genome replication during scAAV vector production. Fewer rescued, replicated, and packaged AAV genomes were detected in the constructs with shRNA cassettes proximal to Wt-ITR, which is consistent with the lower vector yields in the purified preparations from these particular scAAV-shRNA constructs (FIGS. 3A and 3C). Both H1-shApob and U6-shFluc cassettes led to the truncation of vector genomes, suggesting that the negative impact on rAAV production is not shRNA sequence-specific (FIG. 3).

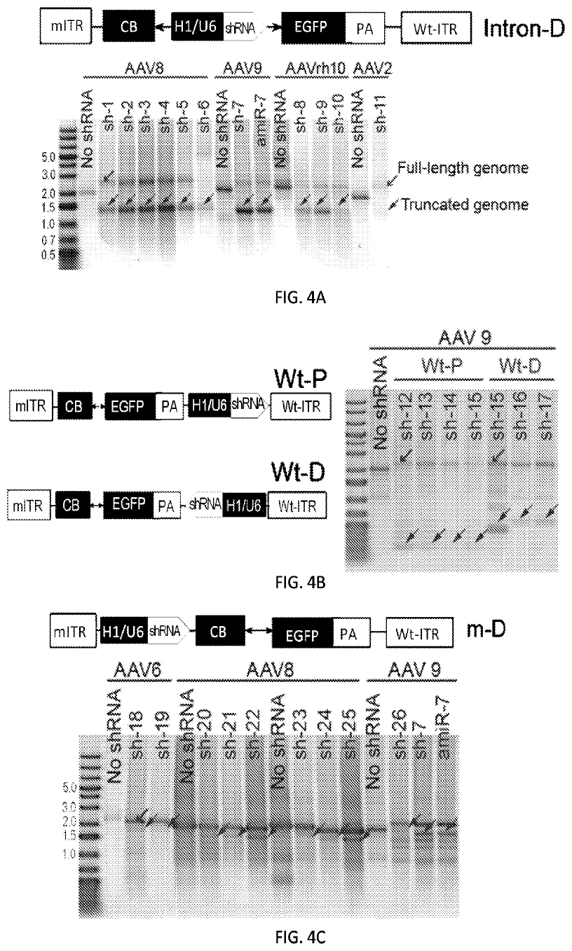

Genomes of scAAV vectors carrying different shRNA cassettes at different positions and packaged with different AAV serotypes were next investigated. When shRNA cassettes were located in the intron between the EGFP gene and CB promoter of scAAV genomes, AAV vectors including AAV8, AAV9, AAVrh10, and AAV2 all generated truncated genomes (FIG. 4A). scAAV genomes containing shRNA embedded into a miR-30 shuttle also produced the shortened genome (FIG. 4A). When shRNA cassettes were cloned into sites distal or proximal to Wt-ITR, shRNA cassettes were found closer to the wild-type ITR generated smaller truncated genomes (FIG. 4B). When shRNA cassettes were positioned distal to a mITR, more intact genomes were found; however there was still a noticeable amount of truncated genomes (FIG. 4C).

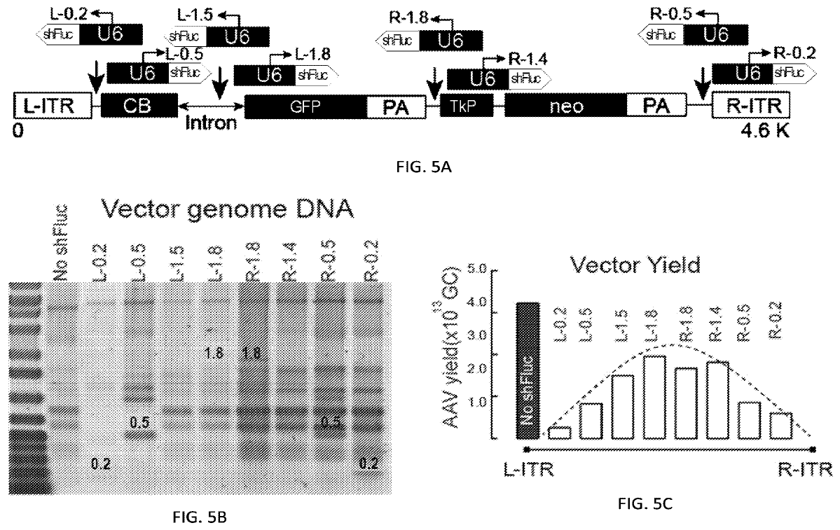

To clarify if only a self-complementary vector genome phenomenon was observed, vector genomes of conventional single-stranded (ss) AAV vectors in purified ssAAVshRNA preparations were examined. Both full length and truncated vector genomes as seen in the scAAV preparations were identified, as well as the negative impact on the yield of vectors with shRNA cassettes close to either 5′ or 3′ Wt-ITR (FIG. 5). Taken together, shRNA cassettes hinder replication of both ss and scAAV genomes and cause vector genome truncations. Both the intact and truncated genomes with a linear single-stranded genome size<4.7 kb are packaged into AAV vectors. Truncation of shRNA cassettes containing AAV genomes is a universal phenomenon, it is not AAV serotype, shRNA cassette, or genome format (ss versus sc) specific.

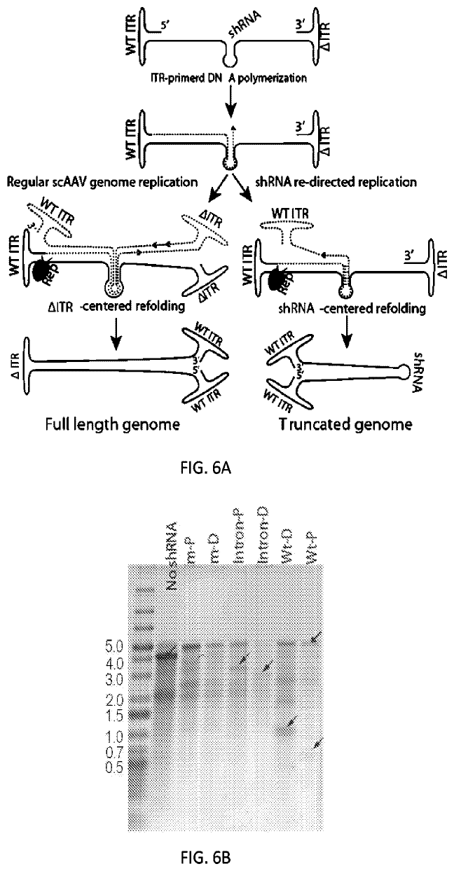

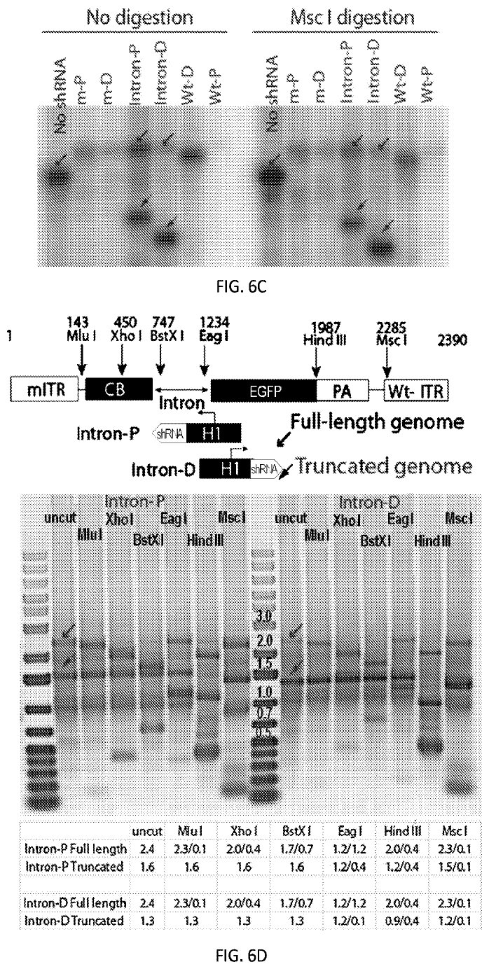

Based on this data, a model illustrating the impact of shRNA cassettes on AAV genome replication was formed (FIG. 6A). Genome replication starts from the Wt-ITR during scAAV vector production and forms an intra-molecular double-stranded DNA with an mITR loop when a normal scAAV genome without a shRNA cassette is used. However, for the scAAV-shRNA construct, when AAV genome replication reaches to the shRNA cassette, the base-paring shRNA stem redirects the orientation of replication and uses the newly synthesized genome as a template to form the truncated genome. If the replication overcomes the complementarity of shRNA's secondary structure, it will generate the full-length scAAV genome for packaging. Therefore, replication of the scAAVshRNA genome has two possible fates: a complete replication to produce a full length genome, or a partial replication to generate a truncated genome. Viral genomes extracted from purified viral preparations were run in an alkaline gel and the sizes of both intact and truncated genomes were found to double (FIG. 6B). The result indicated the truncated genome is an intra-molecular double-stranded DNA-like scAAV genome at a smaller size (FIG. 6B). The Southern blot analysis of the viral genomes with and without digestion with an Wt-ITR-specific restriction enzyme confirmed the truncated genomes contain an EGFP fragment and that the Wt-ITR is where the replication starts (FIG. 6C).

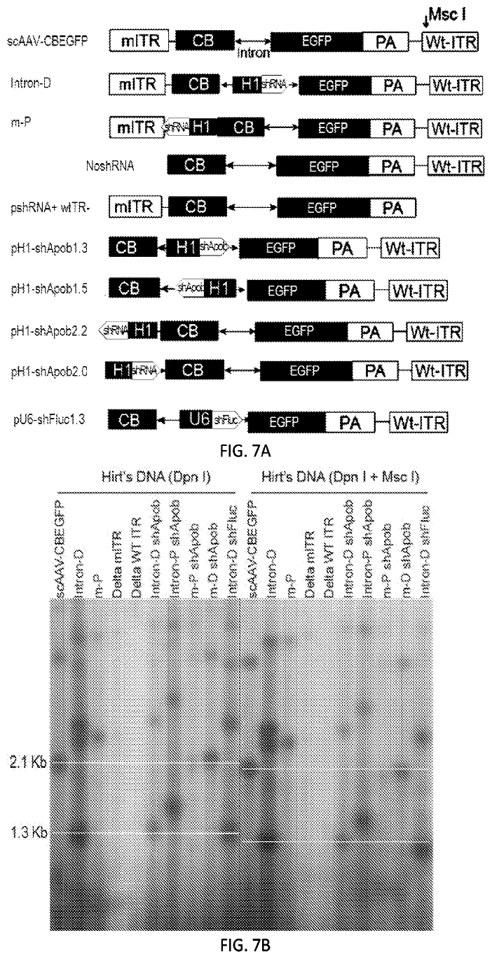

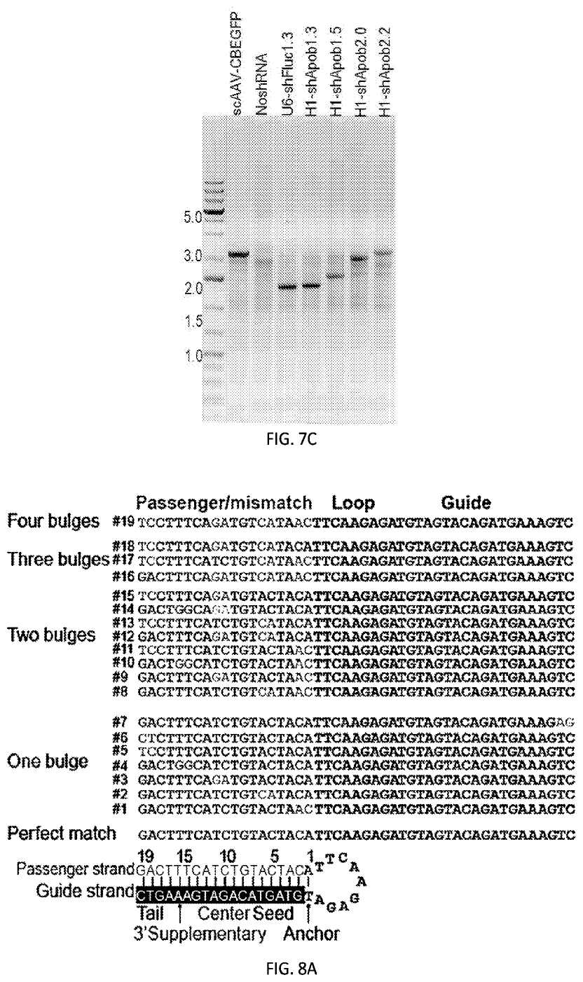

To further characterize the truncated AAV genomes, restriction enzyme mapping was performed on the DNA from a scAAV9 vector carrying the shApob in the intron. Three restriction enzymes (Mlu I, Xho I, and BstX I) with reorganization sites upstream of shRNA-encoding DNA only digested full-length AAV genomes, but the other three restriction enzymes (Eag I, Hind III, and Msc I) which recognize the downstream shRNA-encoding DNA region can digest both full-length and truncated genomes (FIG. 6D). The digestion results suggest the shRNA sequence is a dividing line for the full-length and truncated genomes. More importantly, the short hairpin DNA seems to serve as another mutant ITR during the AAV genome replication. To test this concept, the mITR was replaced with a DNA fragment encoding shApob or shFluc in the scAAV constructs (FIG. 7A). When the hybrid shDNA-ITR plasmid was co-transfected with adeno helper plasmid and Rep/Cap trans-plasmid, the rescued AAV genomes could be detected from Hirt's DNA (FIG. 7B), which was confirmed by large scale rAAV production and purification (FIG. 7C). In summary, a DNA fragment with a hairpin structure can serve as an alternative mutant ITR for rAAV vector production.

### Example 2: Development of Efficient and Safe rAAV Compatible Silencing Construct

Reports show that AAV-delivered shRNAs may cause cellular toxicity by saturating the RNAi machinery. To overcome this issue, scientists have embedded antisense RNA into endogenous miRNA scaffolds to improve small RNA processing and reduce toxicity. However, the artificial miRNAs are not as potent as shRNAs in gene silencing. The principle of artificial miRNA design is to replace the natural miRNA with the desired antisense RNA and to keep approximately 100 bases of flanking sequences at both ends.

It is therefore necessary to design rAAV-compatible molecules for efficient, safe, and sustained in vivo gene silencing. Example 1 demonstrates a strategy to overcome the negative impact of shRNA cassettes on the vector genome replication and homogeneity and yield of AAV vectors. This example provides data demonstrating the advantages of replacing currently utilized artificial miRNAs (AmiRNAs), which harbor a shRNA stem sequence consisting of 100% complementary passenger and guide strands, with a novel design that mimics the natural structures of native miRNAs (i.e. having reduced complementarities between passage and guide strands). The new design is more compatible with rAAV genome structures and AAV replication biology, leading to a more homogenous rAAV-AmiRNA genome population from the rAAV production process.

After screening and characterizing a panel of rAAV vectors carrying 14 different pri-miRNA structures for the homogeneity of rAAV genome populations, nine pre-miRNA structures, namely miR-21, miR-375, miR-30a, miR-26a, miR-451, miR-33, pri-miR-99, pri-miR-194, and pri-miR-155 were selected as the AmiRNA backbones to create a panel of mouse Apob specific AmiRNAs. The selected AmiRNAs were tested for their silencing efficiency and As-RNA processing in vitro in comparison with the classic shRNA design. The constructs were also packaged in small and large scale rAAV production and their ratios of truncated to full length vector genomes were compared. When the leading constructs were tested in vivo, it was found that the novel AmiRNA design can achieve the same silencing efficiency as the classic shRNA design.

Design and Generation of rAAV Compatible shRNA Expression Cassettes

The base pairing in the shRNA stem appears to be critical for the AAV genome replication. Lowering the thermodynamic stability of the DNA fragment that encodes the shRNA improves AAV genome integrity.

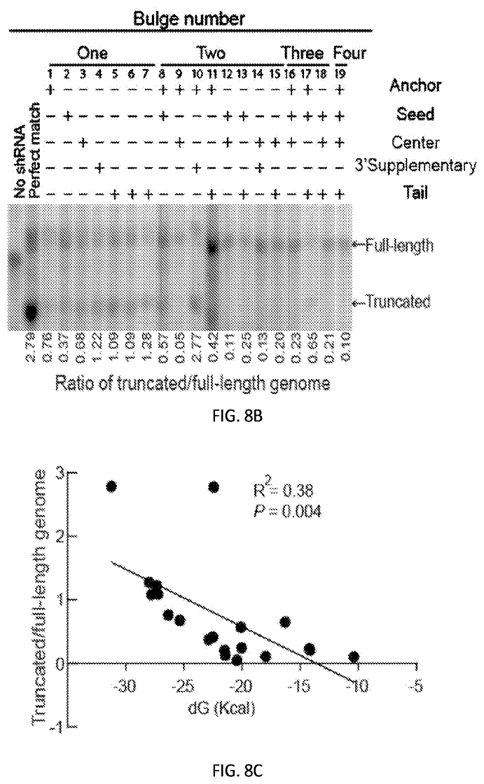

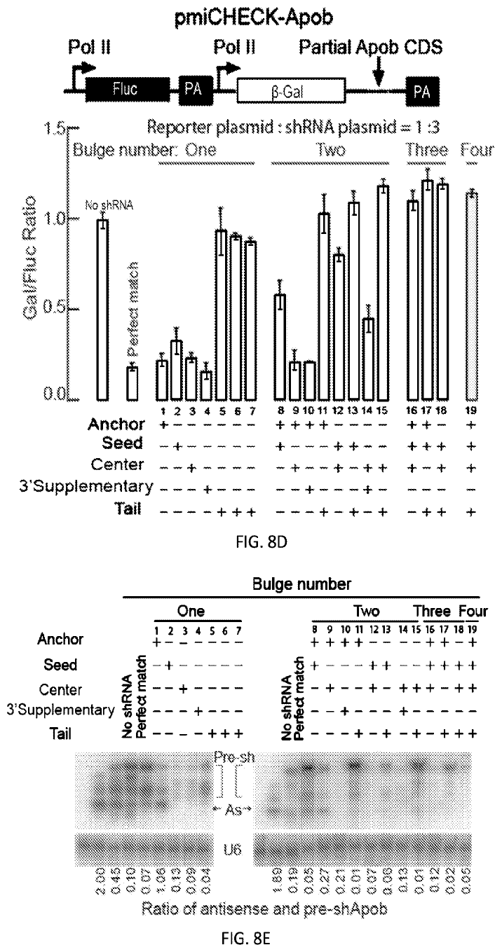

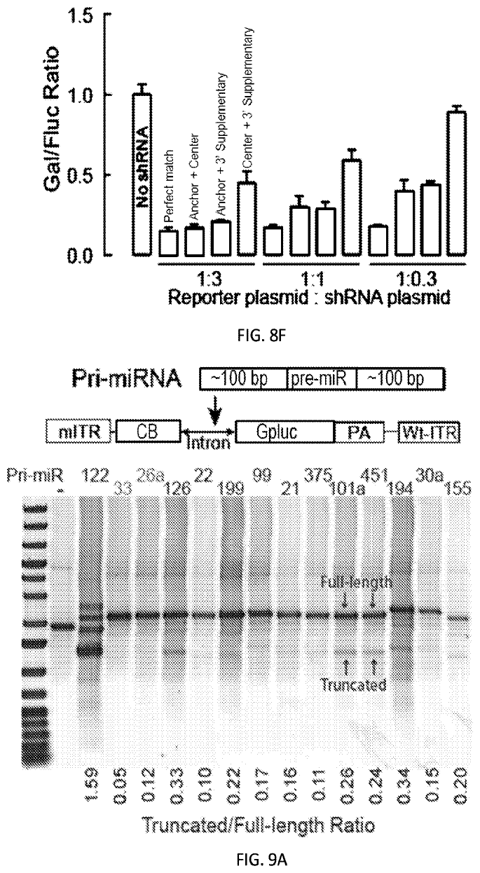

This phenomenon was examined by keeping the guide strand of shApob unchanged and introducing one to four bulges at different positions in the passenger strand (FIG. 8A). The shApob cassettes carrying bulges were incorporated into the intron between the EGFP and CB promoter in scAAV genome plasmids. The scAAV-shApob plasmid was co-transfected with pAdeno-helper plasmid and pRep2/Cap9 plasmid into 293HEK cells, and it was found that the truncated genomes in shApob with bulges are significantly less than a perfect match with shApob, except for one outliner (FIG. 8B, lane 10). The shApob constructs with lower thermodynamic stability correlate with less truncated genomes (FIG. 8C). To quantitatively compare the gene silencing efficacy of the shApobs carrying bulges in the passenger strand, they were co-transfected with the pmiCHECK-Apob sensor plasmid, which contains part of the Apob cDNA fragment targeted by the shRNAs in the 3′UTR of the Gal reporter gene (FIG. 8D). Among the scAAV plasmids that generate much less truncated vector genomes (FIG. 8D), shApob carrying bulges at the anchor and center achieved a silencing effect comparable to the shApob with a perfectly matched stem (FIG. 8D, lane 9). However, the small RNA Northern blot analysis showed massive unprocessed pre-shApob from the bulged-shApob as compared to conventional shApob (FIG. 8E). It was also determined that the silencing effect from bulged-shApob is not as potent as the conventional shApob when lower doses of shApob plasmids are transfected according to the reporter gene sensor assay (FIG. 8F).

Artificial miRNAs mimicking the natural miRNA structure are as potent as conventional shRNAs in target gene silencing, but more compatible with rAAV genomes for efficient, safe, and sustained in vivo gene silencing

As demonstrated by the above, lowering the shRNA thermodynamic stability by introducing bulges in the passenger strand reduced the portion of truncated genomes in rAAV preparations, but the gene silencing capability was greatly compromised as compared to the classic shRNA design. To improve pre-shRNA processing, the Apob antisense RNA was embedded into miRNA scaffolds which use the endogenous RNAi machinery. First, a panel of 14 rAAV-pri-miRNA expression constructs was screened, and the impact of natural pri-miRNAs which contain bulges in their stem on the scAAV genome integrity was analyzed.

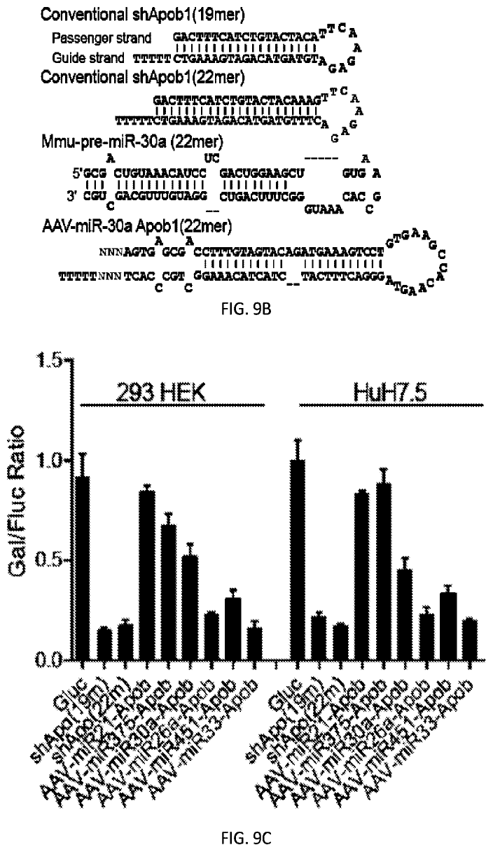

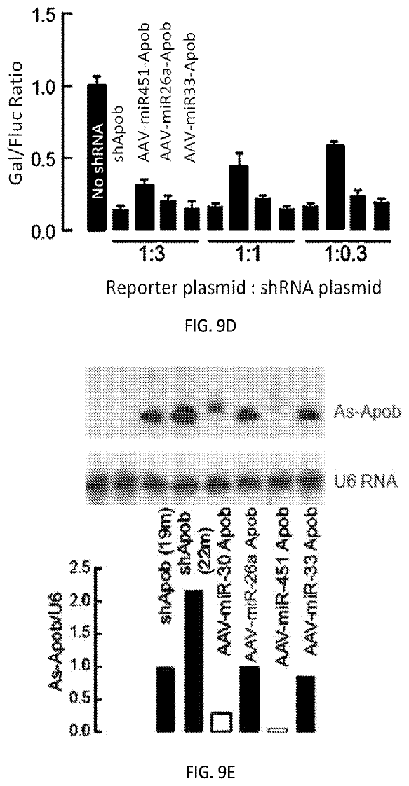

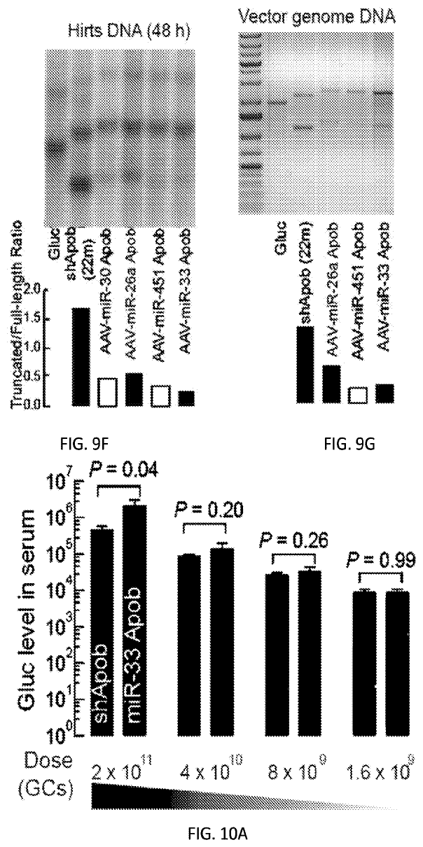

Overall, all endogenous pri-miRNAs expressing rAAV constructs also generated truncated vector genomes but the proportions of the truncated vector genomes were smaller than those in rAAVshRNA constructs. Some pri-miRs such as pri-miR-33, pri-miR-26a, and pri-miR-22 generated minimal truncated genomes; however, rAAV pri-miR-122 gencrated approximately the same amount of truncated genomes as rAAVshRNAs, likely due to the high complementarity between the passenger and guide strands of the miR-122 stem sequence (FIG. 9A). This observation suggests that the current principles in the AmiRNA design, including formation of perfect, 100% pairing between the passenger and guide strands in the stem sequence, is incompatible with rAAV replication biology and may not be suitable for rAAV-mediated in vivo gene silencing. This observation has led to a novel design concept for rAAV-compatible AmiRNAs.

Second, pri-miR-21, pri-miR-375, pri-miR-30a, pri-miR-26a, pri-miR-451, pri-miR-33, pri-miR-99, pri-miR-194, and pri-miR-155 were selected as scaffolds to embed the Apob antisense. To mimic the native structures of corresponding pri-miRs, the stem sequence of the miRNA was replaced with the Apob shRNA guide strand and bulged passenger strand as naturally present in the original pri-miRNA (FIG. 9B). In addition, the flanking sequences were arranged as those in the natural pri-miR structure (FIG. 9B). The RNAi efficacy of those miRNA scaffolds carrying the Apob antisense RNA were compared with the conventional shApob in 293HEK and Huh7.5 cells (FIG. 9C). Using the novel AmiRNA design, even when the ratio between the miRNA scaffolds and Apob sensor plasmid were lowered by one log, the miR-33 and miR-26a scaffolds still showed robust gene silencing capability (FIGS. 9C and 9D). No pre-Apobs were detected by small RNA Northern blot (FIG. 9E). The amounts of mature antisense Apob RNAs from these two scaffolds are comparable with the conventional shApob construct (FIG. 9E). The constructs were packaged into AAV9 vectors in small and large scale vector production, and fewer truncated forms of viral vector genomes in both crude Hirt's DNA and purified viral preparations were found (FIG. 9F and 9G).

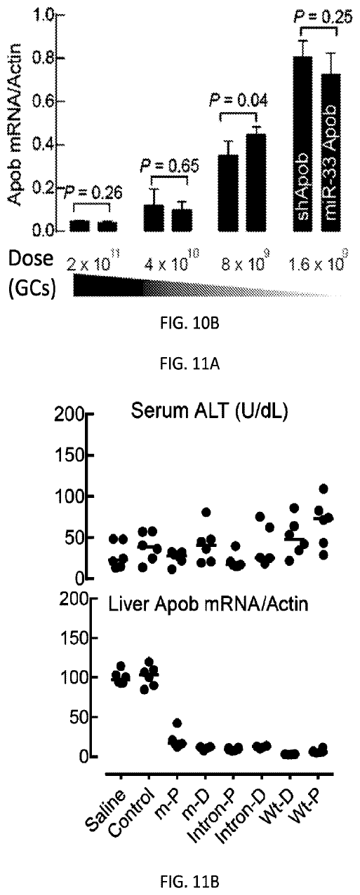

The silencing efficiencies of those novel rAAV-AmiRNAs in vivo and the classic rAAV-shRNA construct were compared. There were improvements in reporter gene expression (i.e., more intact vector genomes) in mice receiving vectors carrying miR-33 Apob as compared to conventional shApob at the dose of 2×10¹¹ (FIG. 10A) and comparable gene silencing effects (FIG. 10B). In summary, studies using natural miRNA scaffolds with lower complementarity in the stem and flaking sequences as the carrier for target specific antisense RNA improve AAV genome integrity and achieve gene silencing capability comparable to conventional shRNAs, but better than the current artificial miRNA design. Further studies are under way to further characterize RNAi machinery involved with the processing of those novel AmiRNAs and evaluate potential toxicity that may or may not be caused by long term expression of those silencing molecules from rAAV etc.

### Example 3: Short DNA Hairpins Function as the Mutated Terminal Repeat of Adeno-Associated Virus Vectors

Truncated AAV Genomes were Found in Mice Received scAAV9-shApob

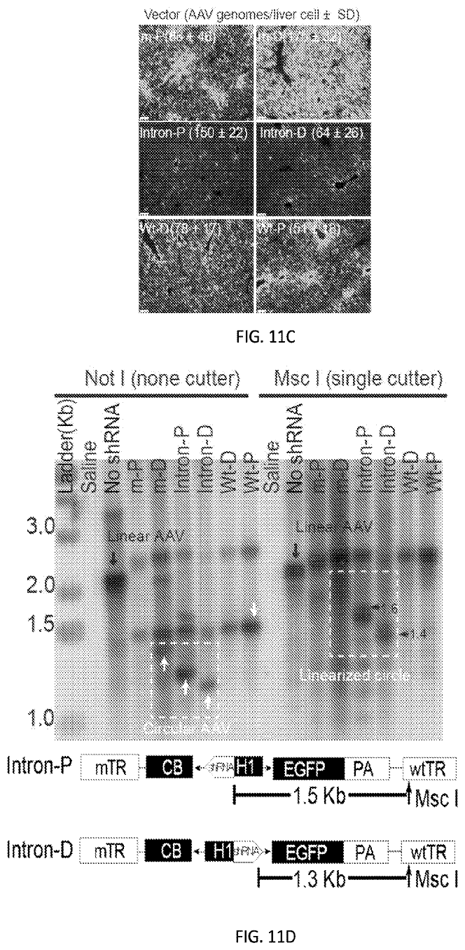

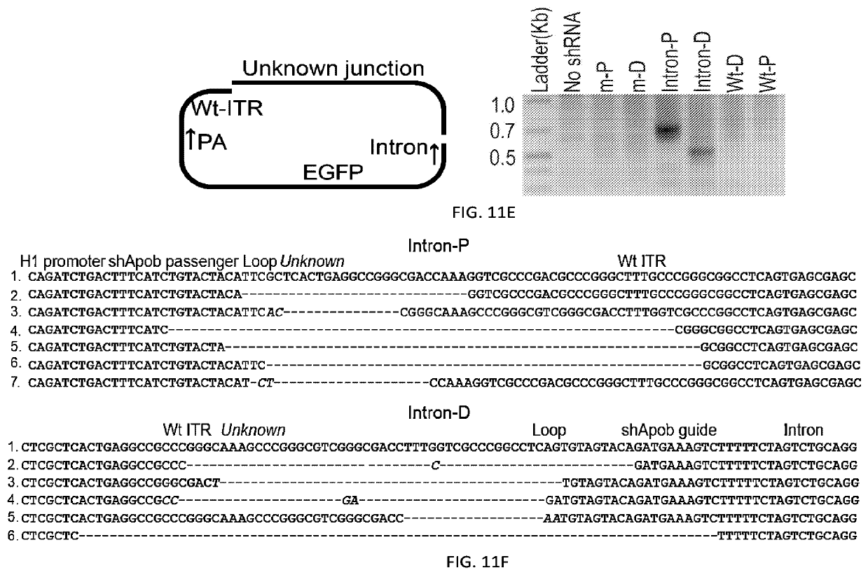

To compare the functionality of scAAV carrying shRNA cassettes in different position, the scAAV9-shApob vectors were administered intravenously with 5×10¹³ genome copies per kg each to adult male C57B/6 mice. The vector titer was determined by Taqman quantitative PCR using EGFP probe¹⁵. Three weeks after the injection, no significant increase was detected in serum alanine aminotransferase (ALT), indicating no AAV-delivered shRNA related liver toxicity (FIG. 11A). Efficient gene silencing was observed in Apob gene in the liver of mice received the scAAV9 carrying shApob cassette at different position shown in FIG. 2A, compared to vector expressing no shRNA or saline control (FIG. 11B). In contrast to EGFP expression from scAAV-shApob plasmids in 293HEK cells, EGFP expression was much lower in the liver of mice received scAAV9 carrying shApob in the intron (Intron-P and Intron-D groups), even the transduced AAV genomes are comparable which was analyzed by Taqman quantitative PCR using EGFP probe (FIG. 11C). AAV vector genome will form linear and circular monomers and concatemers which have different transduction potency after vector metabolism in cells¹². To characterize the molecular structures of AAV genomes in liver, Southern blot analyses was performed. Total liver DNA was digested with Not I which does not cut the AAV genome and Msc I which is a single cutter in AAV genome, respectively. In the Not I digested liver DNA, a probe binding to the EGFP transgene detected not only the linear and circular AAV molecules at expected size but also smaller molecules in mice received scAAV9 carrying shApob in the intron. After Msc I digestion, the small molecules migrated up, indicating the small molecules are in circle. The sizes of linearized bands are 1.5 kb and 1.3 kb equal to the distance from wtTR to the location of shApob (FIG. 11D). Results indicate that small circular molecules consist of EGFP transgene and wtTR. To explore the unknown junction with wtTR, PCR primers targeting the upstream of EGFP and downstream of wtTR were designed which can only amplify circular DNA template (FIG. 11E). From the genome DNA from mice received scAAV9 carrying shApob in the intron, the fragments were amplified at expected sizes (FIG. 11E), cloned into TOPO vector and sequenced them (FIG. 11F). The sequence data showed the junction to wtTR in Intron-P treated mouse is the sequence of shApob passenger strand and H1 promoter and the junction to wtTR in Intron-D treated mouse is the sequence of shApob guide strand and intron (FIG. 11F). In these two different truncated AAV molecules, the EGFP transgene is in the lack of Chicken β-actin (CB) promoter which explains the lower EGFP expression. The results suggest the shApob cassettes lead to the AAV genome truncations and compromise the EGFP reporter gene expression in vivo.

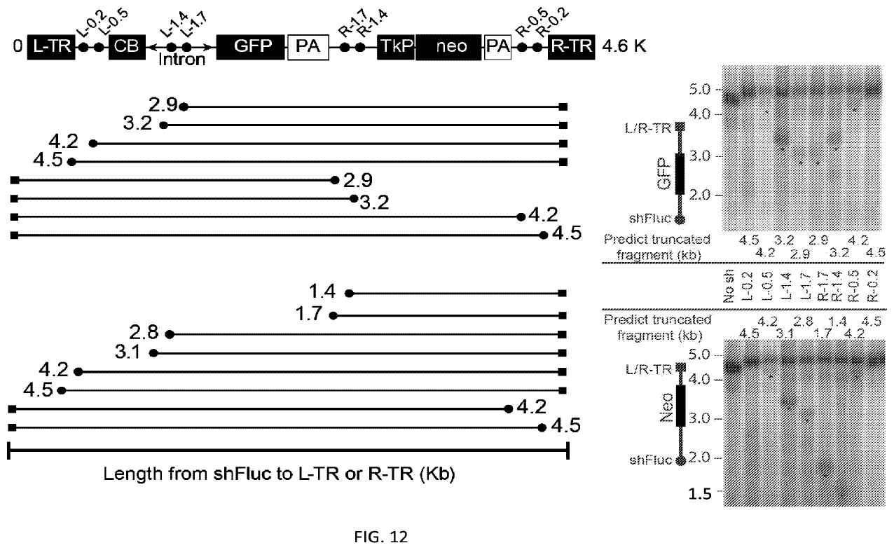

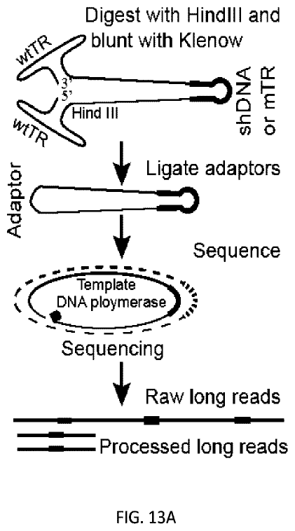

To clarify if what was observed is not only a self-complementary vector genome phenomenon, the Hirt DNA from HEK293 cells transfected with pAd, pRcp/Cap and conventional single stranded AAV vector (ssAAV) plasmids harboring shFluc-encoding DNA at different locations (FIG. 12) were also examined. Different from scAAV, the replication of ssAAV can start from left TR or right TR. After the hybridization with GFP and Neo probes, all the truncations except the 4.5 kb fragments (FIG. 12) were detected. No detection of these 4.5 kb fragments might be due to their small size difference with 4.6 kb full-length genome in regular agarose gel. This Southern blot data further confirmed that shRNA-encoding DNA is a barrier of genome replication for both ssAAV and scAAV.

### Short DNA Hairpins Function as the Mutated Terminal Repeat

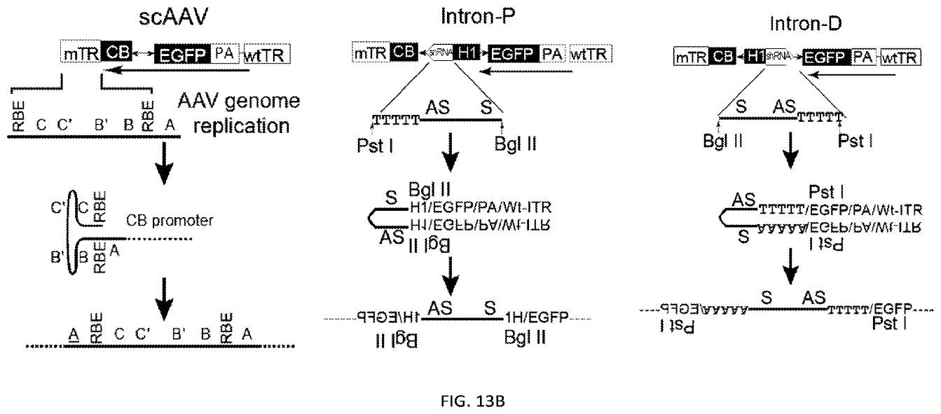

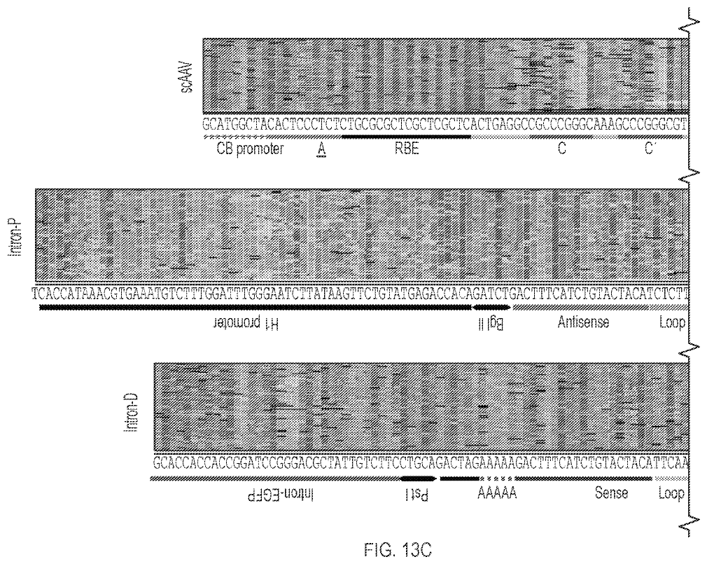

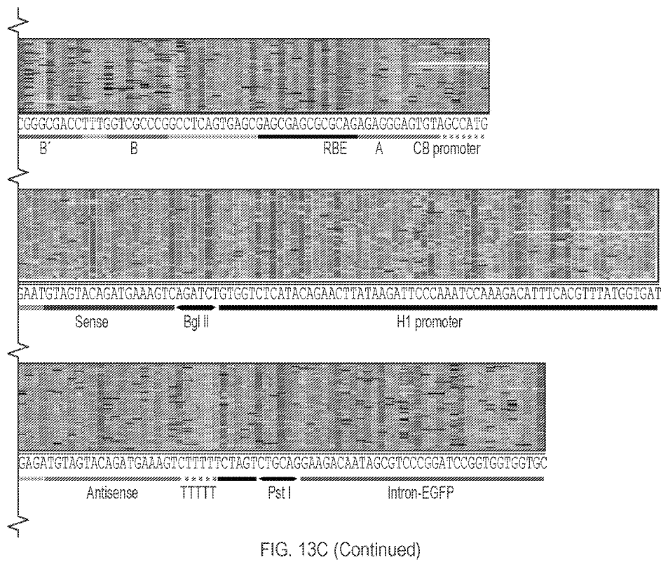

A model to illustrate how short DNA hairpins impact AAV genome replication (FIG. 6A) is provided. In a normal scAAV genome without shRNA cassette, its genome replication starts from the wtTR and forms an intra-molecular double-stranded DNA with mTR as a loop. However, for the scAAV construct bearing short DNA hairpin, when AAV genome replication reaches to the hairpin, the base-paring of the hairpin stem switches the template from parental strand (FIG. 6A, solid line) to the daughter strand (FIG. 6A, dotted line). As a consequence of redirected genome replication, truncated genome will be produced. If the replication overcomes the complementarity of hairpin structure, it will generate the full-length scAAV genome for packaging. In both cases, the Rep will nick the wtITR to release the newly synthesized genomes for next round of replication. Viral genomes extracted from purified viral vectors were examined in an alkaline gel and the sizes of both intact and truncated genomes were doubled (FIG. 6B). The results indicate the truncated genome is an intra-molecular double-stranded DNA like scAAV genome at smaller size (FIG. 6B). To characterize the truncated AAV genomes, restriction enzyme mapping was performed on the DNAs from two scAAV9 vector carrying shApob in the intron (Intron-P and Intron-D). Three restriction enzymes (Mlu I, Xho I and BstX I) with reorganization sites upstream of shRNA-encoding DNA only digested the full-length AAV genomes, but the other three restriction enzymes (Eag I, Hind III and Msc I) which recognize the downstream of shRNA-encoding DNA can digest both full-length and truncated genomes (FIG. 6D). The result showed the shRNA sequence is a dividing line for the full-length and truncated genomes. Taken the alkaline gel and restriction enzyme mapping data together, the truncated genomes are intra-molecular double-stranded DNA with shRNA at one end. To further characterize the truncated molecules, they were sequenced by single molecule real-time sequencing (SMRT, Pacific Biosciences) platform. In standard SMRT library preparation, adaptors will be added to both ends of one DNA molecule form a circular template for sequencing. In library preparation, adaptor is added to one end of the intra-molecular DNA. To avoid the potential sequencing difficulty from the strong secondary structure of wtTR at the end, the viral genome DNA with Hind III was digested to remove the wtTR fragment and performed SMRT-CCS (FIG. 13A). After sequencing, the adaptors were removed from the raw long reads and the processed long reads will be the sequence of denatured AAV genomes. Because of lacking of the Rep nicking sites in the mTR, scAAV genome continues its replication after mTR, forms molecules with mTR in the middle and complementary sequences at two ends, and generates intra-molecular double-stranded genomes after folding back (FIG. 13B left). Based on the model, in Intron-P vector, when the genome replication reaches to the antisense strand of shRNA, the base-pairing from the shRNA stem re-directs the orientation of replication, the five thymine and Pst I site right after shRNA antisense strand will not the replicated and the sequence of Bgl II site and HI promoter located before the shRNA sense strand will be duplicated in the truncated genomes (FIG. 13B middle). In Intron-D vector, the genome replication turns back before the Bgl II site which is next to the shRNA sense strand, the Bgl II site will be not replicated, but the five thymine and Pst I site will be replicated (FIG. 1B3 right). In the scAAV-CBEGFP plasmid, there is only one “A” site in the inner border of mTR. But in the scAAV-CBEGFP vector genome, one more “A” (A) after RBE site was found, indicating the re-directed and continued genome replication by mTR. It is the first time to sequence the mTR loop in the scAAV vector since it has been developed (FIG. 13C top). In the sequencing data, the molecules with predicted hairpin DNA centered structures (FIG. 13C middle and bottom) were also detected. The truncated AAV genomes are intra-molecular double-stranded DNA with short hairpin DNA in the middle.

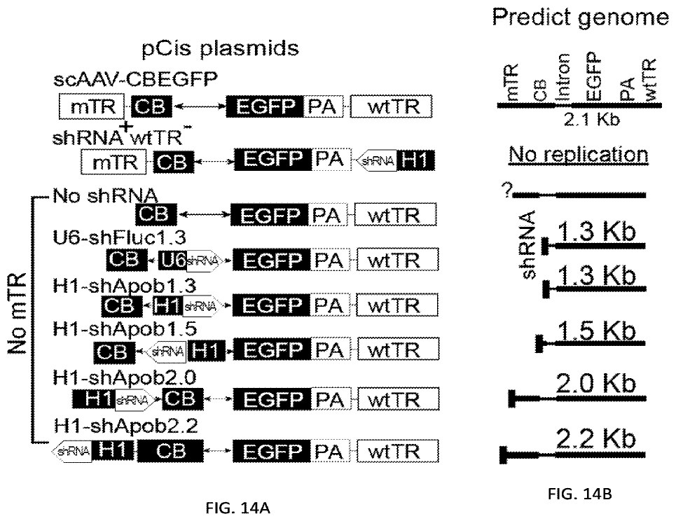

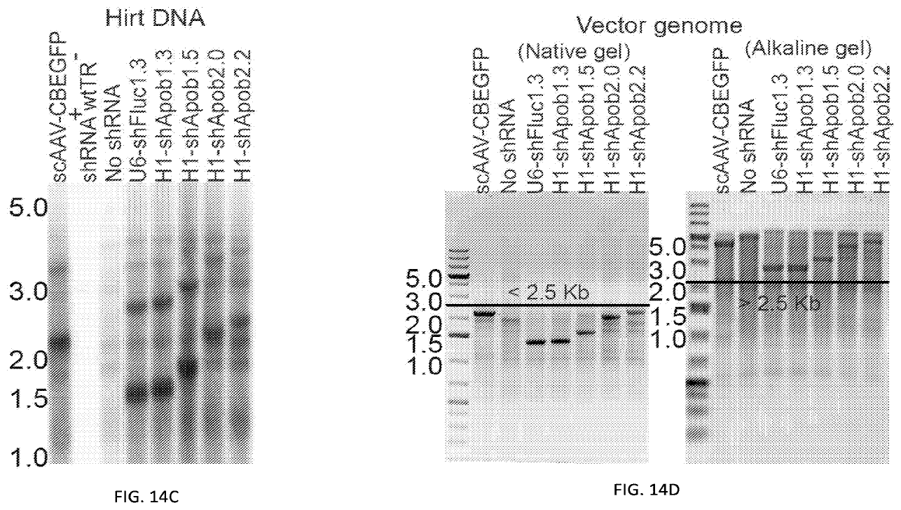

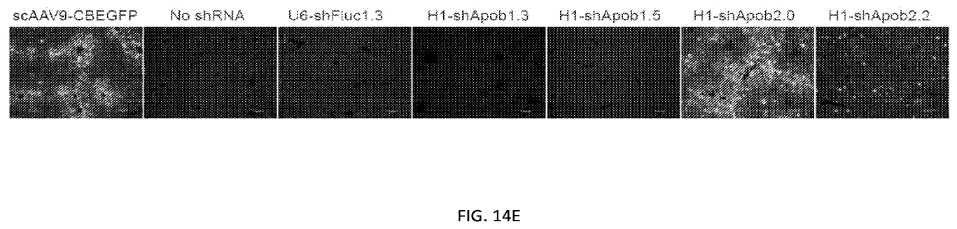

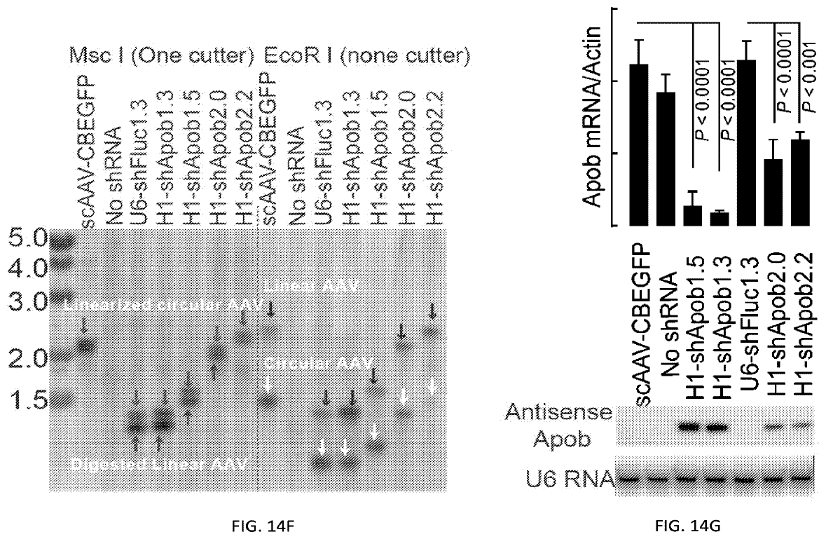

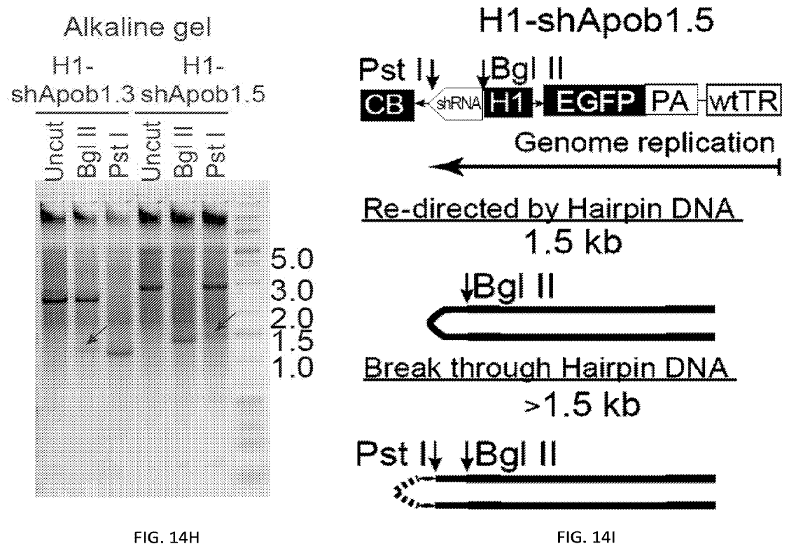

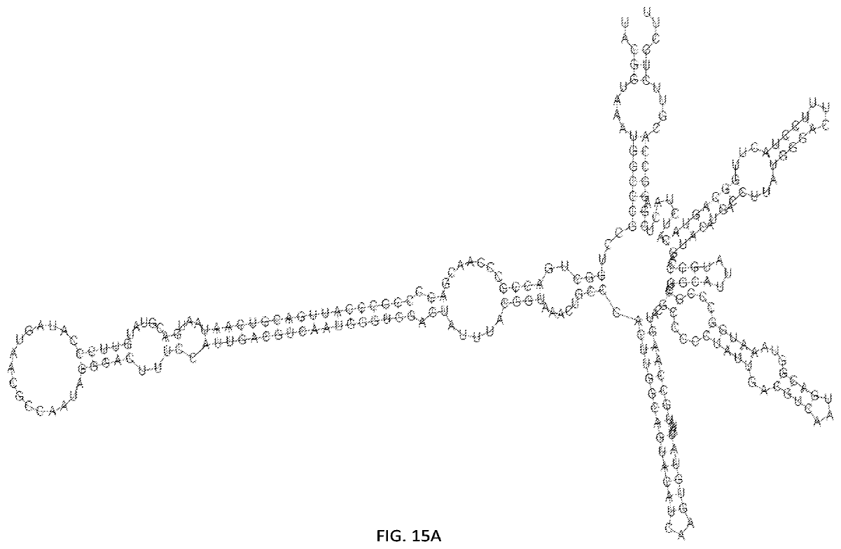

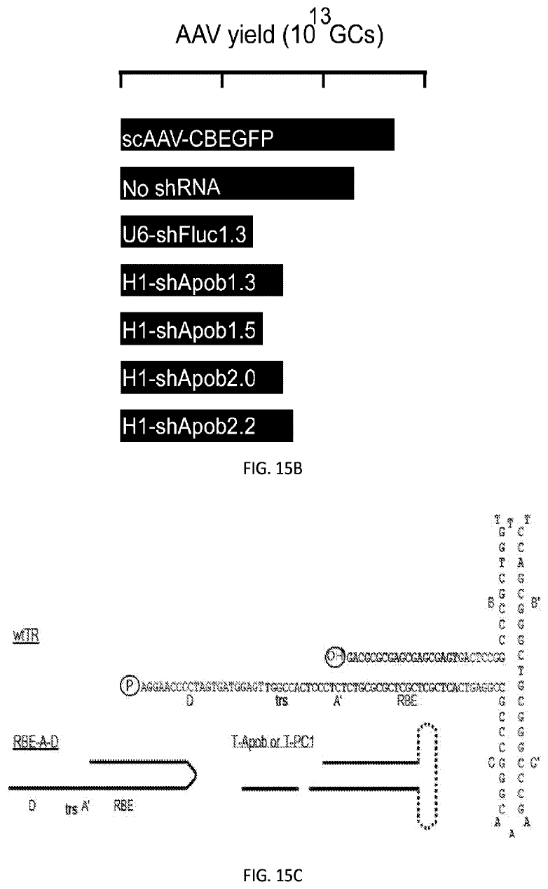

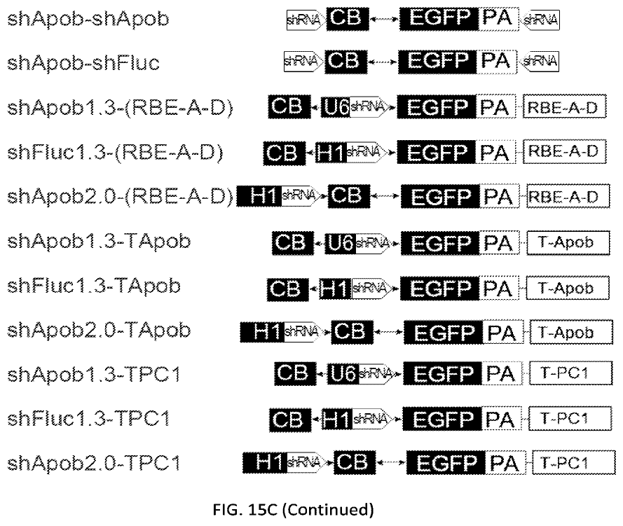

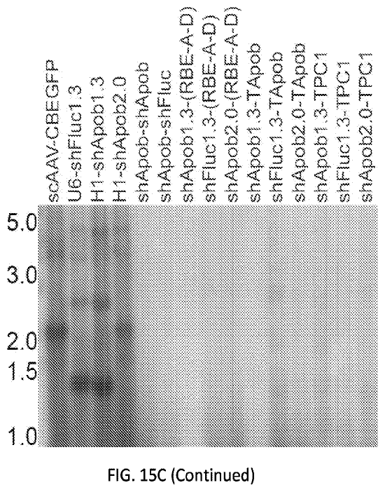

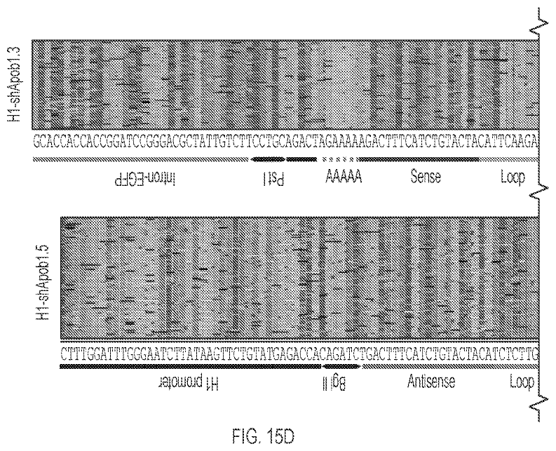

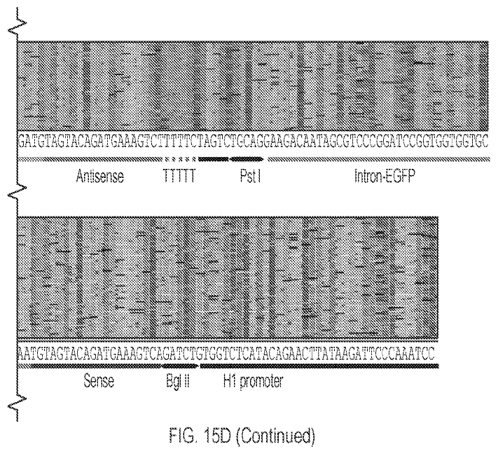

Results indicated that short hairpin DNA at least functions as an alternative mTR in the truncated AAV genomes. To further characterize, the mTR was replaced with DNA fragments encoding shRNA against Apob or Fluc gene in the scAAV constructs (FIG. 14A-14B, showing constructs and predicted lengths). In the absence of mTR, and the presence of wtTR and hairpin DNA (U6-shFluc1.3, H1-shApob1.3, H1-shApob1.5, H1-shApob2.0 and H1-shApob2.2), AAV genomes can be rescued and existed as monomers and dimers (FIG. 14C). The genome can be rescued from construct carrying only one wtTR (pmD⁻). The sequence which can form hairpin structure within the CB promoter may serve as mTR for the genome replication (FIG. 15A). When wtTR was replaced with hairpin DNA (pshRNA⁺wtTR⁻), no AAV genomes were able to be rescued from the triple-transfected HEK293 cells (FIG. 14C). The original elements (D, RBE, trs and A) were observed in the wtTR and maintain the same T-shape structure by replacing the B-B′ and C-C′ with the other palindromes, no AAV genome can be rescued either (FIG. 15B). Then these pCis plasmids were packaged into AAV9 and the purified rAAV genomes in both native and alkaline gels were analyzed. The molecular weight of AAV vectors containing wtTR and hairpin DNA at two ends was doubled in alkaline gel comparing to the size in native gel, indicating the vector genomes are also intra-molecular double-stranded DNA like scAAV genome (FIG. 14D). The vector yield is comparable to the scAAV control (FIG. 15C). SMRT sequencing revealed the symmetrical structure of AAV genome, hairpin DNA in the center and complementary sequences at two sides (FIG. 15D). These AAV vectors were named shAAV.

To test their functionalities in vivo, shAAV9 was intravenously injected carrying EGFP gene into adult C57/B6 mice at the dose of 1.6×10¹³ GCs/kg and harvested liver tissues 3 weeks later. Because of the lack of CB promoter for EGFP reporter gene in the viral genomes, U6-shFluc1.3, H1-shApob1.3 and H1-shApob1.5 shAAVs produced few green cells in the liver. Compared to regular scAA VEGFP vector, the H1-shApob2.0 shAAV vectors achieved comparable EGFP transduction efficacy, but the EGFP expression was much less in the H1-shApob2.2 shAAV. To characterize the molecular forms of shAAV in vivo, the same Southern blot analysis as FIG. 11C was performed. The Southern blot data showed shAAV vectors exist as both linear and circular forms like scAAV vectors in vivo. Dominant AAV molecular H1-shApob2.2 is in linear form may be the reason of the low EGFP expression in vivo (FIG. 14F). ApoB gene expression was down-regulated by shAAV vector carrying shApob cassette and the RNAi phenomenon was confirmed by small RNA Northern blot (FIG. 14G). These results are unexpected because the shRNA cassettes in the shAAV vector genomes are not intact to produce functional shRNA (FIG. 4g and FIG. 15D). Based on the SMRT sequence data, both H1 promoter and passenger strand RNA-encoding sequence are missing in H1-shApob1.3 shAAV genome. Also there are no five thymine terminal signal and guide strand RNA-encoding DNA in the H1-shApob1.5 shAAV genome (FIG. 15D). To validate the SMRT sequence result, the H1-shApob1.3 and H1-shApob 1.5 shAAV vector genomes were digested with Bgl II and Pst I and checked the size in the alkaline gel. The size of uncut H1-Apob1.3 shAAV genome became 2.6 kb because of the denaturing of the intra-molecular double-stranded DNA in alkaline gel. Based on the SMRT sequence data, Pst I digests the middle of H1-Apob1.3 and the size of Pst I digested genome should remain as 1.3 kb. The Bgl II digested genome should be doubled because there is no Bgl II site in the H1-Apob1.3 genome. The Pst I digestion confirmed the presence of Pst I site in the genome, but except the dominant 2.6 kb fragment from Bgl II digestion, the additional fragment (>1.3 kb), indicating the Bgl II digested some of shAAV genomes (FIG. 14H) was seen. In H1-Apob1.5 shAAV, there is one Bgl II site and no Pst I site in the genome based on the SMRT sequence data. The Bgl II site was confirmed and the extra >1.5 kb fragment in the Pst I digestion (FIG. 14H) was also found. In the AAV package, except producing the dominant shAAVs, the AAV genome replication broke through the hairpin barrier and generated intact shRNA expression cassettes (FIG. 14I and FIG. 6A). Then the PolII promoters were deleted for the shRNAs in the shAAV plasmids, packaged them into AAV9 vectors and inject the mice again. After 3 weeks of the injection, neither the reduction of Apob gene nor the Apob antisense in the liver of mice was detected. The EGFP expression and AAV molecular forms were not affected by the deletion of Pol III promoter.

### Materials and Methods

### Vector Design, Construction, and Production

The shFluc fragment in pRNA-U6.1/Neo-siFluc (GenScript, Piscataway, NJ) was integrated into the MluI, PpuMI and Bbs I site of pscAAVCBEGFP plasmid to generate pscAAV-shFluc plasmids bearing shFluc in different locations. And also the shFluc fragment was cloned into pUF11 plasmid at the Kpn I, SgrA1, Xho I and Bbs I sites to generate pUF11-shFluc serial plasmids. The mutant TR in pscAAVCBEGFP was deleted by Pac I and Mlu I digestion to pmTR⁻ plasmid. The pshRNA⁺wtTR⁻ was made by replacing the Msc I-Pac I fragment in wtTR with shApob-encoding DNA. Pac I and Mlu I digestions was also used to delete the mTR from the original plasmids of pU6-shFluc1.3, pH1-shApob1.3 and pH1-shApob1.5. ShApob-encoding DNA was incorporated into the Sal site of pmTR− to generated plasmids pH1-shApob2.0 and pH1-shApob2.2. The RBE-D-A, T-shApob and T-PCI adaptors were cloned between the Pac I and Msc I sites of wtTR to reconstruct the wtTR. To delete the H1 promoters from pshAAV plasmids, Bgl II and BstX I fragment was removed from p pH1-shApob1.3, pH1-shApob1.5, pH1-shApob2.0 and pH1-shApob2.2 plasmids. The shFluc fragment was integrated into the BamH I of pmTR⁻ to make pshFluc 1.3 plasmid without U6 promoter. Partial Apob cDNA was amplified from mouse liver RNA and incorporated between the Not I and Xho I site of pmiCHECK to generate shApob activity sensor plasmid. Vectors used in this study were generated, purified, and titered as described²¹. All the constructs will be deposited to Addgene.

### Vector DNA Analysis

Viral DNA was extracted from purified vector following the protocol for extraction of recombinant adenovirus genomic DNA. Vector DNA equivalent to 0.1−1×10¹¹ genomes was loaded into agarose gel or alkaline gel and stained with SYBR gold.

### Southern Blot Analysis for Hirt DNA and Liver DNA.

Low molecular weight Hirt DNA extracted from triple-transfected Hek293 cells and digested with Dpn I before hybridization. To analyze the AAV genome in mouse, three microgram of total liver DNA was digested with EcoR I (none cutter) or Msc I (single cutter) for hybridization. The results were visualized using a FLA-7000 Imager (FUJIFILM). All the probes were labeled by P³² using random primer labeling kit (Takara).

### SMRT Sequencing and Data Analysis

Vector DNA was digested with Hind III to remove the wtTR and agarose gel purified. Around 500 ng viral DNA was submitted for SMRT sequencing. Library preparation and sequencing were done following standard Pacific Biosciences protocols PacBio raw reads processed into circular consensus (CCS) reads using the PacBio pipeline. CCS reads were aligned to the reference sequence using Bowtie. Data was visualized using IGV. Sequence data are available from the NCBI Short Read Archive (www. ncbi.nlm.nih.gov/sites/sra) as GSExxxx.

### Mouse Studies

Male C57BL/6 mice (Harlan, IN) were obtained and maintained and all animal procedures performed according to the guidelines of the Institutional Animal Care and Use Committee of the University of Massachusetts Medical School. After injection of the vectors at indicated dose, the mice were sacrificed 3 weeks later and liver was harvested for cryosectioning using a Nikon TE-2000S inverted microscope. Serum samples were collected and analyzed for ALT using a COBAS C 111 analyzer (Roche Diagnostics, Lewes, UK). Total liver RNA was extracted using Trizol (Invitrogen). qRT-PCR and small RNA Northern blot were performed as reported before²³. rAAV genome copy numbers in total liver DNA were determined.

Statistical analysis

All results are given as mean +standard deviation and compared between groups using the two-tailed Student's t-test.

### Example 4: Short DNA Hairpins Generate Self-Complementary Adeno-Associated Virus Genomes by a Template-Switching Mechanism

Placement of shDNA Sequences Proximal to the Wild-Type TR Reduces scAAV Vector Yield

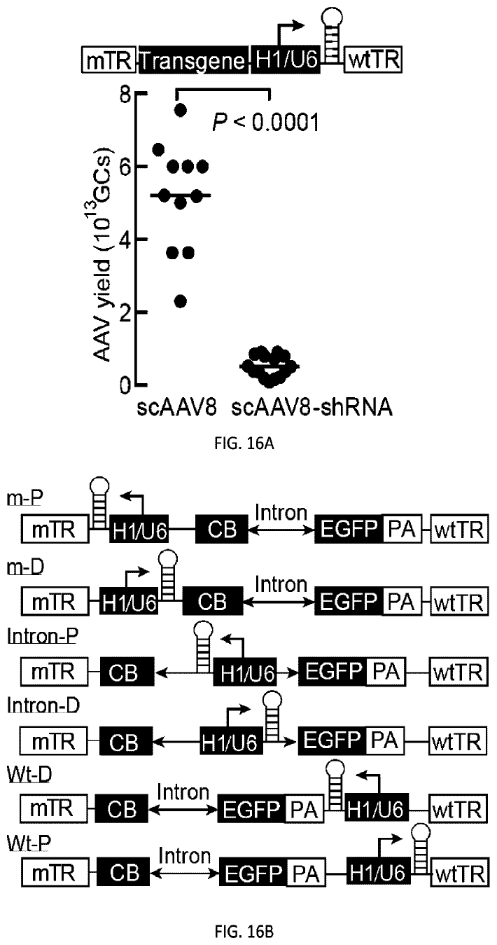

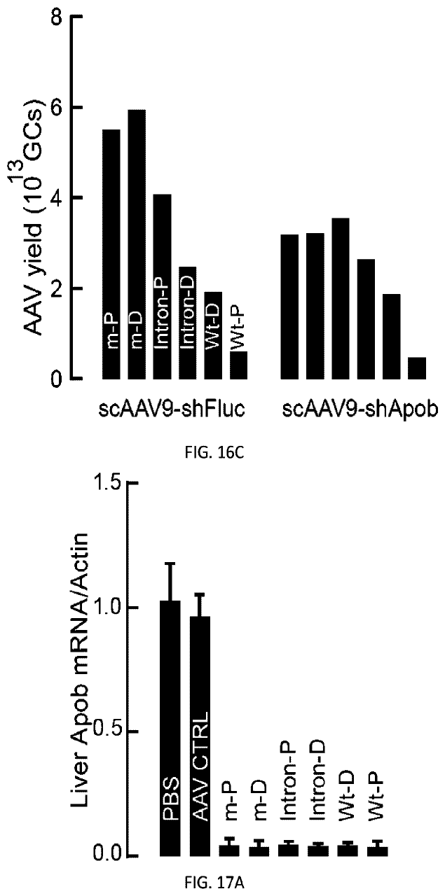

During the manufacturing of scAAV vectors, it was found that the yield of scAAV vectors carrying shRNA expression cassettes proximal to the wild-type terminal repeat (wtTR) was consistently lower than that of scAAV vectors without shRNA cassettes. This difference occurred independent of transgene or shDNA sequences (FIG. 16A). Since the replication of scAAV genomes can only initiate from the wtTR, due to a lack of replication initiation sites in the mTR, whether the hairpin structure of the shDNA sequence interferes with AAV genome replication when placed proximal to the wtTR resulting in poor vector yield was investigated. scAAV vectors that consist of an eGFP reporter gene driven by the CMV enhancer/chicken β-actin promoter (CB) and an shRNA cassette placed at different positions along the scAAV genome were produced. By using two different shRNA expression cassettes, the first encoding an shRNA against mouse Apob driven by the HI promoter (H1-shApob), and the second encoding an shRNA against firefly luciferase driven by the U6 promoter (U6-shFLuc) (FIG. 16B), it was observed that the yield of scAAV-shRNA vectors is reduced when shRNA cassettes are proximal to the wtTR (Wt-P) (FIG. 16C).

Truncated Vector Genomes are Produced from In Vivo Gene Transferred rAAVs Containing shDNA

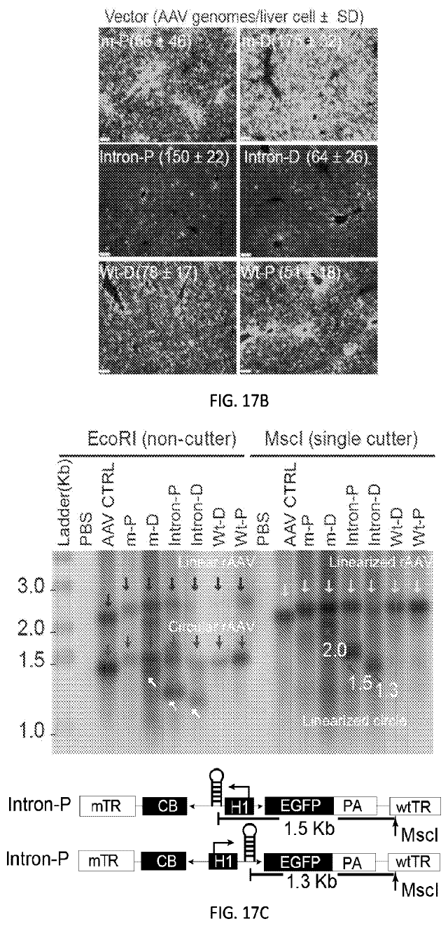

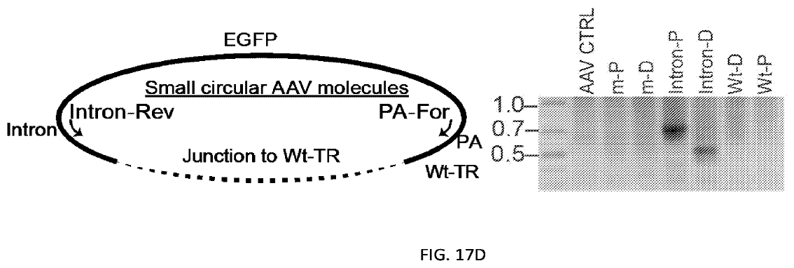

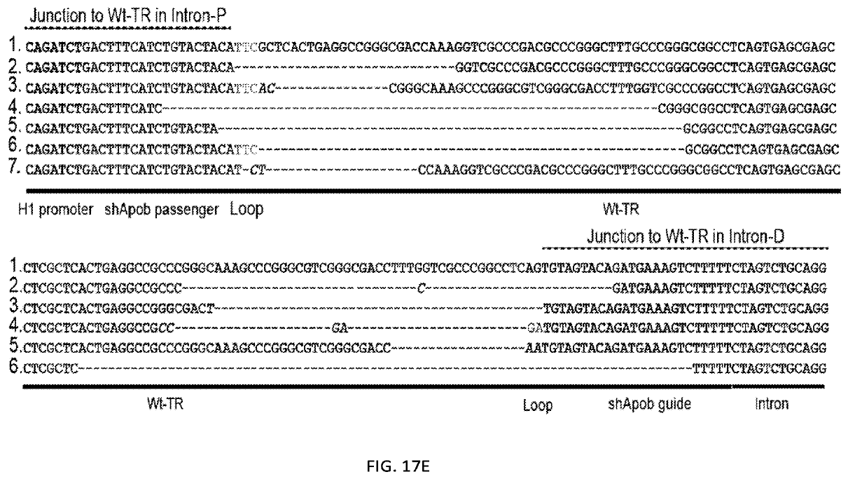

RNAi efficacies and EGFP reporter gene expressions of scAAVs carrying shApob cassettes at different positions were compared in mouse liver. Three weeks after vector infusion, similar levels of Apob gene silencing were observed with all six vectors (FIG. 17A). Despite treating with equal dosages and detecting comparable vector genomes after transduction, mice treated with scAAV9carrying shApob cassettes within the intron (Intron-P and Intron-D) produced much lower EGFP levels compared to other groups (FIG. 17B). In contrast, scAAV vector plasmids all displayed uniform and robust expression of EGFP when transiently transfected into HEK293 cells. To understand the cause for low EGFP expression in Intron-D and Intron-P treatment groups, the vector genomes of treated mouse livers were characterized by Southern blot analysis. While both linear and circular monomers were detected at their expected sizes in liver DNA digested with EcoRI (which does not cut the rAAV genome), additional smaller bands were also observed with m-D, Intron-P, and Intron-D vector treatment (FIG. 17C, arrows). After digesting with MscI, which cuts the rAAV genome once, circular monomers of all vector genomes co-migrated with their linear counterparts (FIG. 17C, arrows). The smaller molecules from the m-D, Intron-P, and Intron-D treatment groups co-migrated up with linear molecules, indicating that these molecules were also circularized. Interestingly, the sizes of the linearized fragments (2.0 kb, m-D; 1.5 kb, intron-D; and 1.3 kb, intron-P) were well correlated with nucleotide lengths ascribed to the distance between MscI sites and the shDNA sequence (FIG. 17C, arrows). These findings suggest that inclusion of shRNA cassettes leads to genome truncations near shDNA sequences.

Southern blot data demonstrate that these smaller molecular forms are circularized vectors that contain EGFP transgenes (detected by an EGFP probe) and wtTR sequences (sensitive to MscI digestion) (FIG. 17C). An inverse-PCR primer set unique to these features was designed to specifically amplify circular DNA templates to query fusion events between shDNA sequences and wtTR regions (FIG. 17D). Sequence analyses of these specific amplicons support the formation of these smaller circularized AAV molecules (FIG. 17E). In the truncated genomes from Intron-P vector treated mice, wtTR was fused with the shRNA guide strand. While in the Intron-D group, wtTR was fused to the shRNA passenger strand. Notably, data show that fusion events in both cases resulted in the loss of the CMV enhancer/chicken β-actin (CB) promoter (FIG. 17E), offering an explanation for the reduction in EGFP expression in the livers of mice treated with Intron-D and Intron-P vectors (FIG. 17B).

Truncation Events Mediated by shDNA Sequences Are Not Specific to AAV Serotype, Sequence Composition, or Position Within the Vector Genome

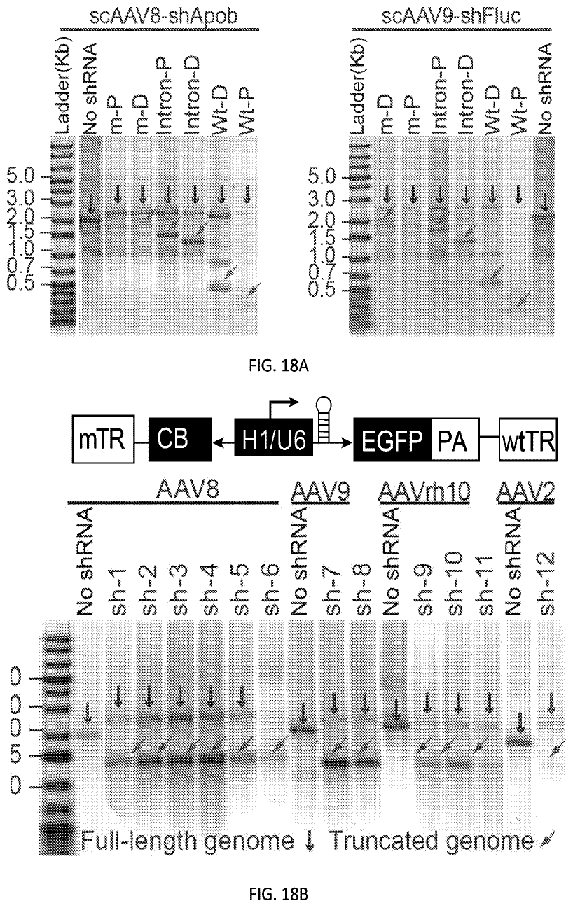

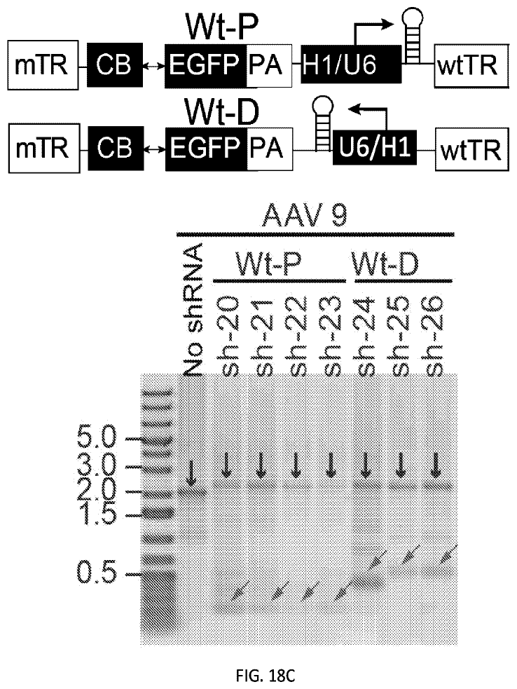

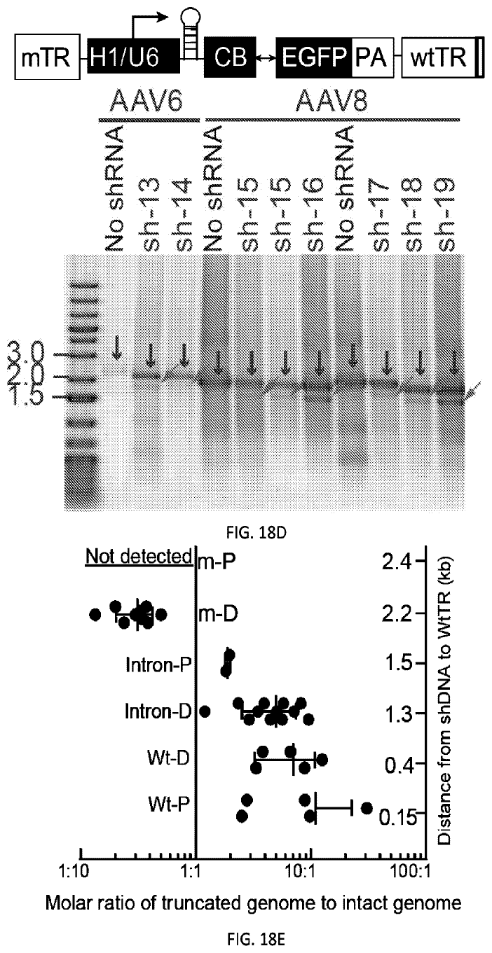

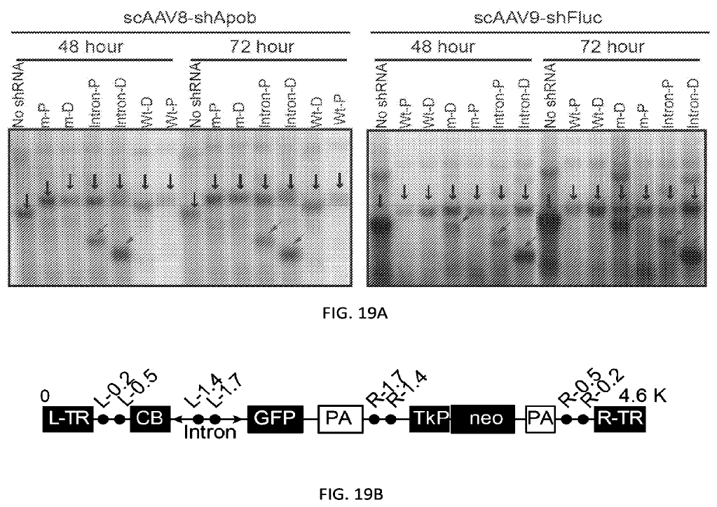

To investigate whether shDNA-associated vector genome truncation occurs during the rAAV production stage or after in vivo transduction, vector DNA from preparations of purified rAAVs was examined. In addition to the full-length genomes, truncated genomes with molecular sizes that correlate well with the nucleotide distance between the wtTR and shDNA sequences were also detected (FIG. 18A). Importantly, the same pattern of genomic species was detected from rAAVs carrying either H1-shApob or U6-shFluc cassettes (FIG. 18A), suggesting that shDNA-associated AAV vector genome truncations are not shRNA sequence-specific.

The position effects of shRNA cassette on truncation frequency was examined: within intronic sequence (FIG. 18B), proximal to the wTR (FIG. 18C), or proximal to the mTR (FIG. 18D). Constructs targeting 26 different genes were packaged into five different capsid serotypes (AAV2, AAV6, AAV8, AAV9, and AAVrh10). All constructs that carrying shRNA cassettes (33 total vector preparations), regardless of serotype or position within the vector genome, generated truncated vector genomes (FIGS. 18B-18D). The sizes of truncated genomes in these preparations correlate with the placement of the shRNA cassette within each vector. Interestingly, the closer the shDNA sequence was to the wtTR, the higher the molar ratio of truncated vector genomes to intact genomes (FIG. 18E). Taken together, data indicate that shDNA sequences drive AAV vector genome truncation in a manner that is independent of serotype, sequence, and position and that one good option for achieving shRNA cassette design compatibility with rAAVs is to place shRNA cassettes proximal to mTR sequences.

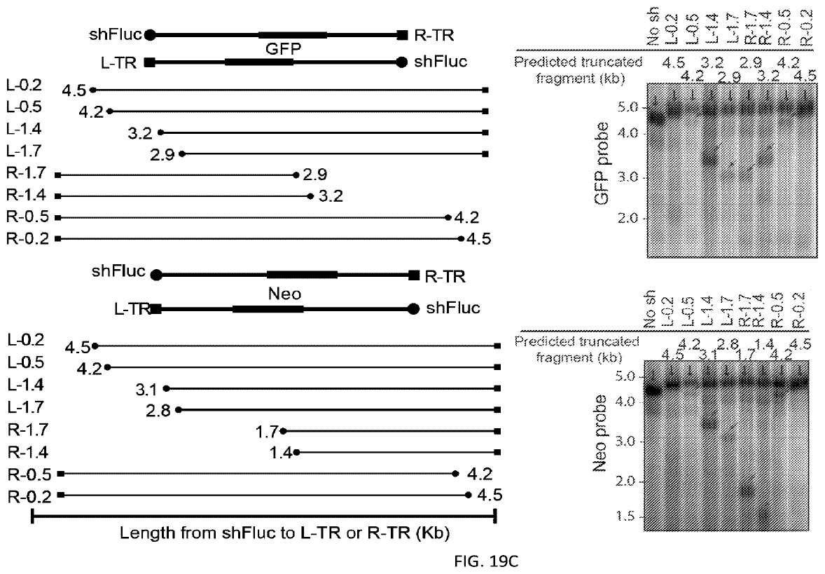

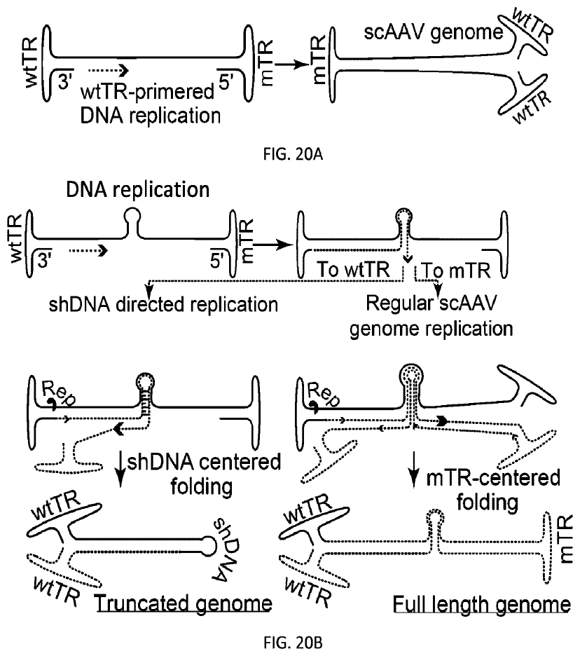

To determine whether genome truncations occur during genome rescue/replication or the packaging phase of viral production, low molecular-weight Hirt DNAs extracted from HEK293 cells after triple plasmid transfection for rAAV production was examined. Southern blot analysis of Hirt DNA revealed detectable amounts of truncated rAAV genomes, suggesting that truncations take place during rAAV genome replication (FIGS. 19A-19B). Notably, fewer rescued and replicated AAV genomes were detected from constructs with shDNA sequences placed next to the wtTR (Wt-P) (FIG. 19A). This observation was consistent with the low vector yields associated with these constructs (FIGS. 19A and 19C). Truncated genomes were also detected in Hirt DNA extracted from cells producing ssAAV vectors harboring shRNA cassettes (FIGS. 19B and 19C).

Short DNA Hairpins Cause rAAV Genome Truncation via Template-Switching During Viral DNA Replication

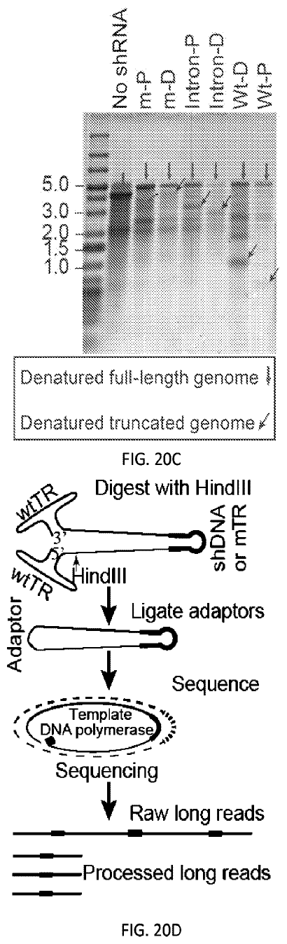

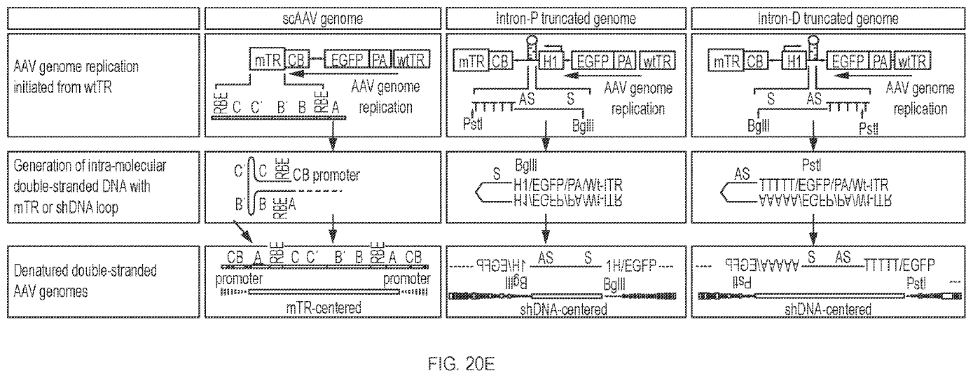

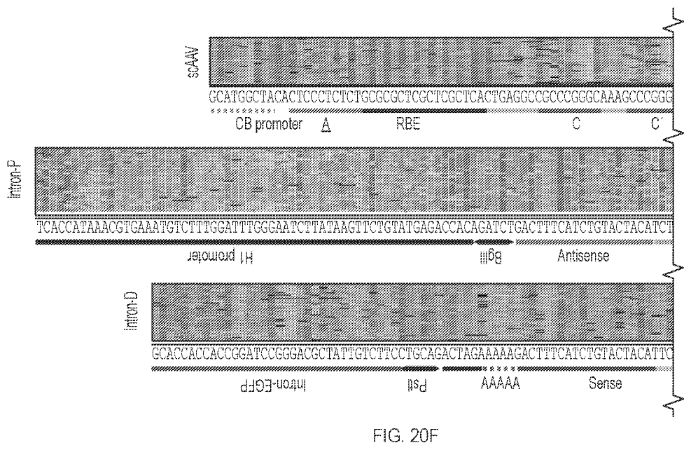

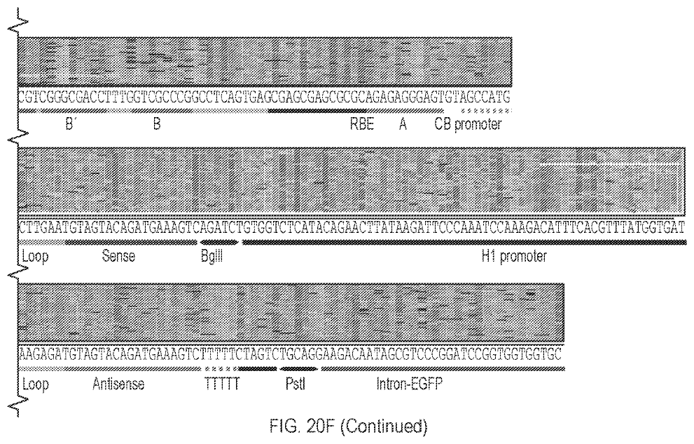

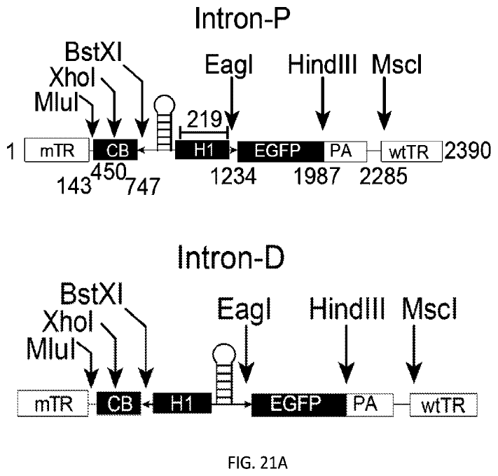

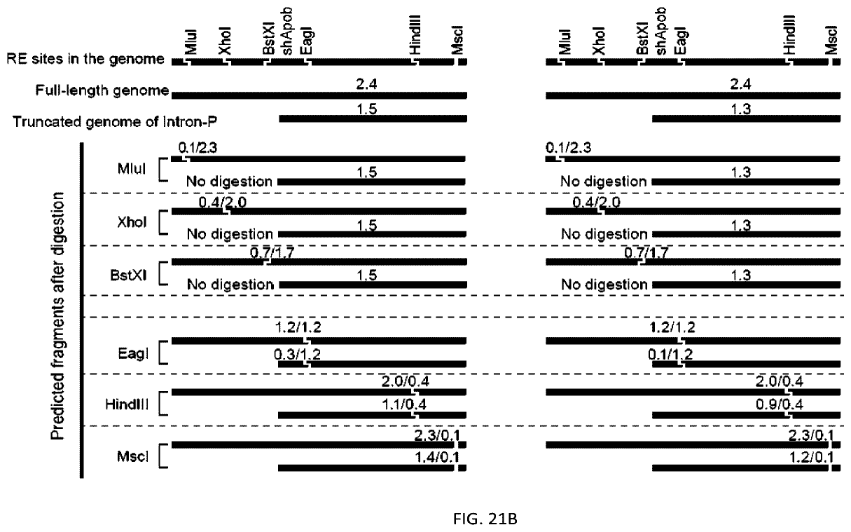

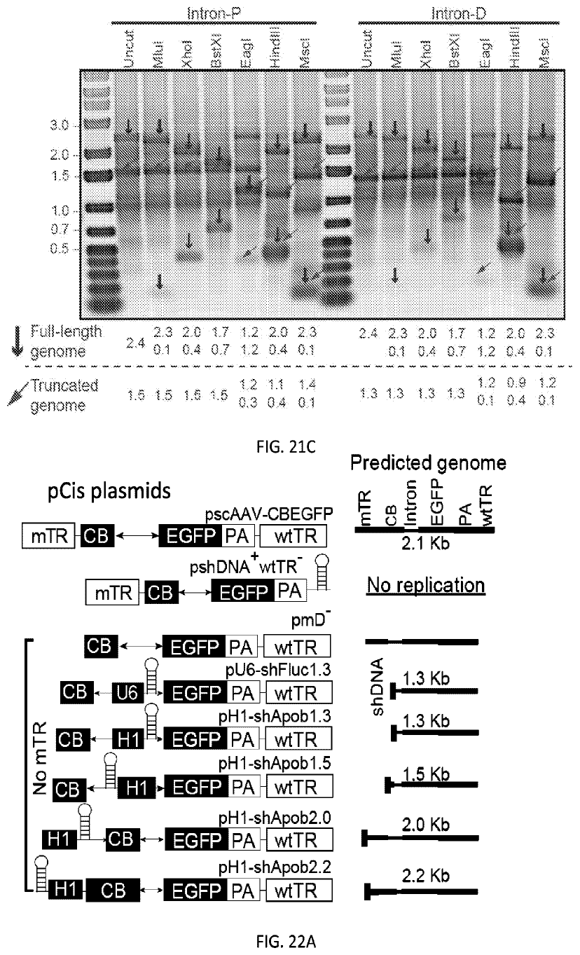

Data suggest that shDNA sequences promote the generation of truncated AAV vectors by impacting viral genome replication. Typically, scAAV replication begins at the wtTR and extends along the length of the rAAV genome. Once replication reaches the mTR, the newly synthesized mTR strand folds into a hairpin, and replication continues with the new strand as template. The resulting intra-molecular, double-stranded DNA consists of an mTR hairpin loop that connects two complementary sequences, each terminating with wtTR ends17 (FIG. 20A). Here it is described that shDNA sequences behave as template switching scaffolds in a manner similar to the mTR region in scAAV vectors (FIG. 20B). rAAV genome replication starts from the wtTR, but faces two choices when reaching the hairpin. If base pairing of the hairpin stem switches templates from the parental strand for replication (FIG. 20B, solid line) to the newly synthesized daughter strand (FIG. 20B, dotted line), then replication makes a U-turn back towards the wtTR without synthesizing sequence beyond the hairpin structure. As a result, truncated, intra-molecular double-stranded genomes with loop regions centered at the shDNA sequence are generated for packaging (FIG. 20B, left). If replication overcomes the complementarity of the hairpin structure, it continues to replicate the parental strand to completion, producing full-length scAAV genomes (FIG. 20B, right). To test this idea, denaturing alkaline-agarose gel electrophoresis was used to examine genomic DNAs extracted from purified viral vector preparations. The sizes of both intact and truncated genomes were doubled as compared to their sizes revealed by native agarose gels (compare FIGS. 18A and 20C), suggesting that the truncated genomes are indeed intra-molecular double-stranded DNA molecules, similar to scAAV genomes. The composition of truncated AAV genomes was examined by restriction enzyme mapping of two scAAV9 vectors that carry shApob cassettes within intronic sequence (FIG. 21). These data indicate that truncated AAV genomes primarily encompass sequence between the wtTR and the shDNA sequence.

High-throughput sequencing was used to analyze the composition of the template switch position. The predicted structure of the self-complementary truncated vector genome is a double-stranded molecule with a single closed end. When the open end of the molecule is adapted using a single-stranded DNA loop, the resulting molecule is a circular single-stranded DNA template, ideal for single molecule real-time sequencing (SMRT). To further improve sequencing processivity, wtTR sequences were removed from vector genomes by digesting viral DNA with HindIII. After purification, the resulting molecules were subjected to single-SMRT-bell adapting to the open end of the truncated genomes to form single-stranded circular templates. The resulting processed long reads, in essence, represent the linear sequences of denatured AAV genomes minus the wtTR regions (FIG. 20D). Vector genomes from scAAV, Intron-P, and Intron-D were sequenced and reads were aligned to custom references based on the predicted outcomes illustrated in FIG. 20E. These references are tandemized forward and reverse strands of vector genome sequence linked together by mTR or shApob hairpin regions (FIG. 20F). Notably, the scAAV-CB EGFP plasmid used in this study contains only one “A” clement at the border of the mTR region (FIG. 20E). During vector production, the A-element (FIG. 20F) was observed to be replicated on the reverse strand, suggesting that the template-switching event occurs at the hairpin terminus. More importantly, the sequences of the shDNA loops within truncated Intron-P and Intron-D genomes (FIG. 20E) are corroborated by SMRT sequencing analysis (FIG. 20F, middle, and bottom panel). In summary, shDNA causes rAAV genome truncation by re-direction of DNA polymerization via template switching during DNA replication. These events generate intra-molecular double-stranded AAV genomes with a terminal shDNA loop. It is worth noting that neither Intron-D nor Intron-P vectors contain intact shRNA expression cassettes. They either lack the antisense strand and the five-thymine termination signal (Intron-P, FIG. 20E middle, and FIG. 20F middle), or the H1 promoter and the sense strand (Intron-D, FIG. 20E right, and FIG. 20F bottom), respectively.

Replacement of the mTR With shDNA Sequences Produces Novel Functional Double-Stranded rAAVs

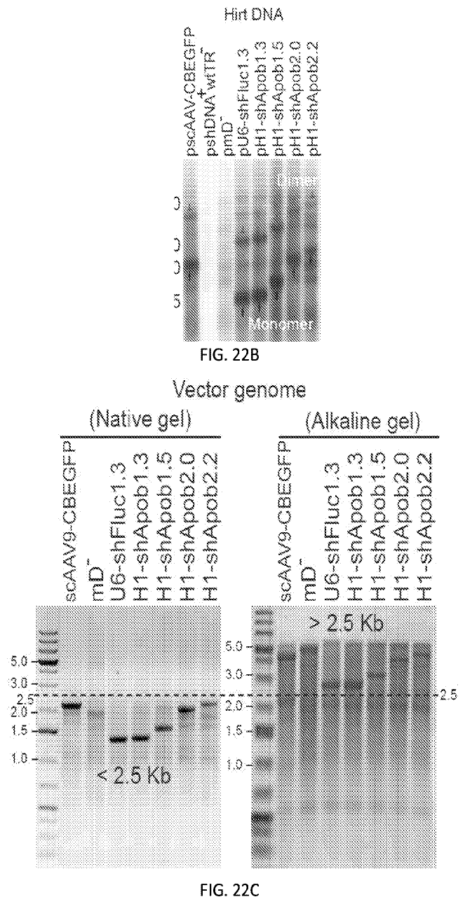

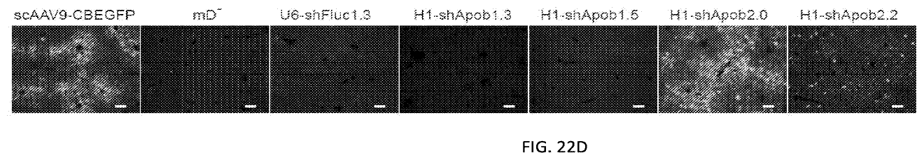

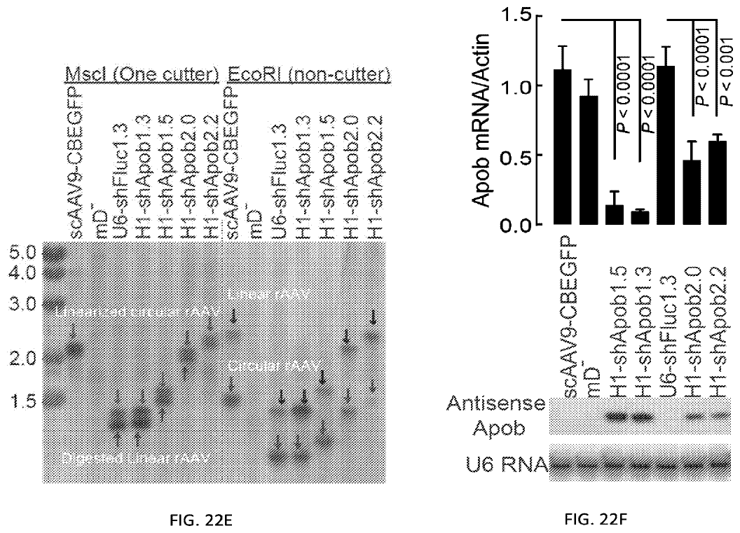

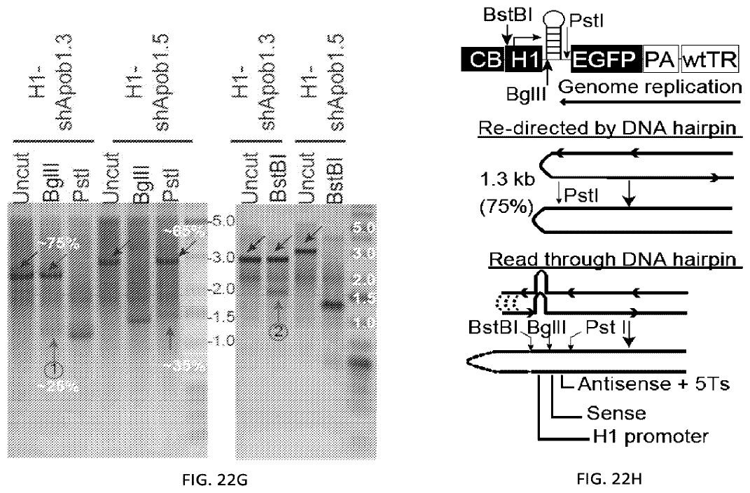

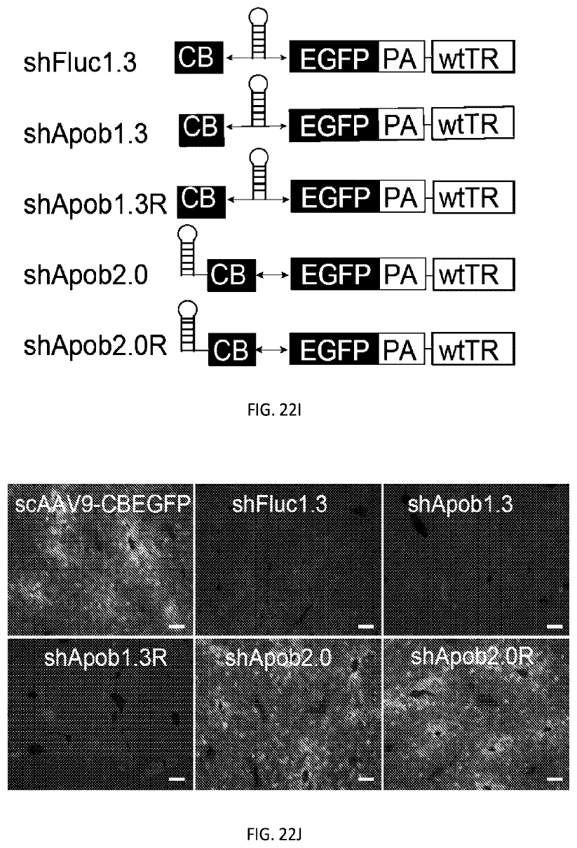

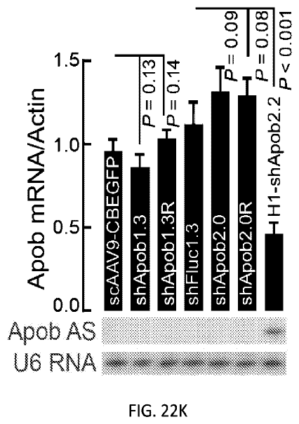

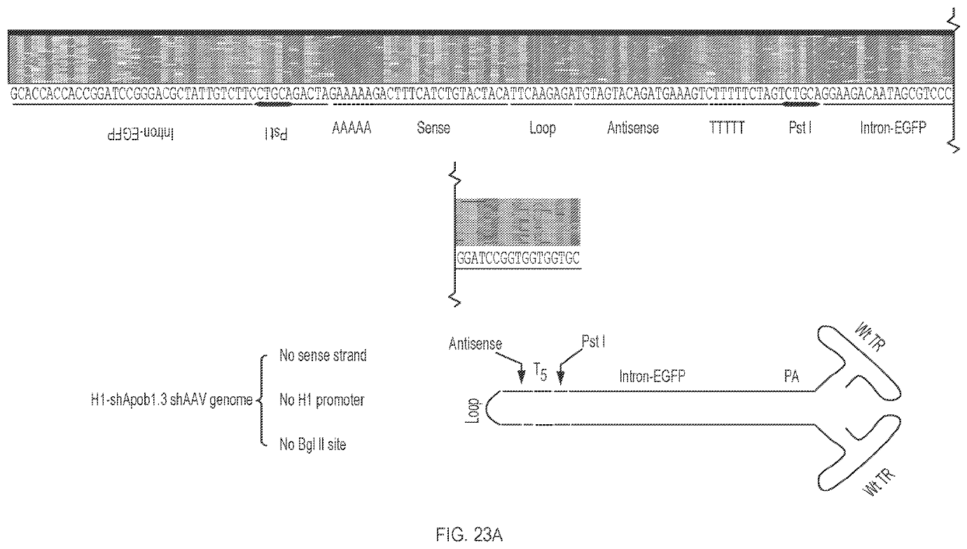

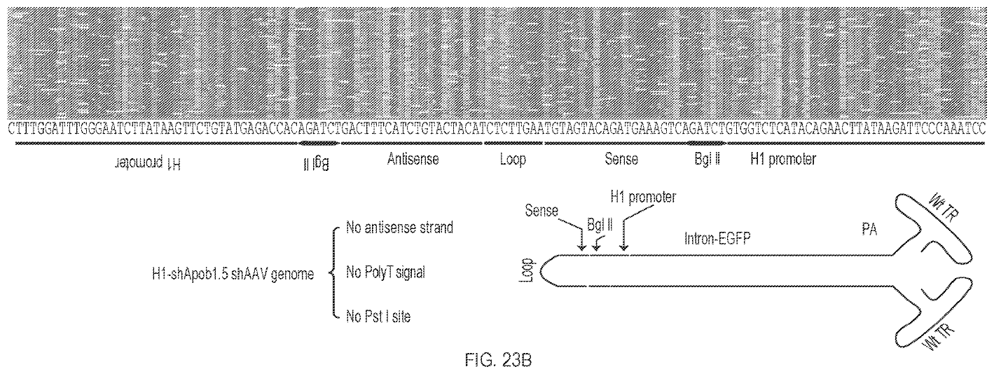

Replacing mTR with shDNA to create a novel AAV vector genome was investigated. The mTR was removed from scAAV constructs containing shRNA cassettes at different positions (FIG. 22A), and evaluated these constructs for in vitro genome rescue and replication, vector production, and in vivo transduction. In the absence of the mTR sequence, scAAV genomes were efficiently rescued from all constructs containing shRNA cassettes (FIG. 22B). However, when the wtTR was replaced with shDNA sequence (pshDNA+wtTR−), no AAV genomes were rescued or replicated (FIG. 22B). The latter observation confirms the importance of the wtTR for AAV replication. Native agarose gel analysis demonstrates that the genome sizes of these vectors produced from constructs in the absence of mTR are equivalent to sequence lengths spanning from the wtTR to the shDNA sequence. The molecular sizes are also doubled in alkaline gels, indicating that these vector genomes are intra-molecular double-stranded DNAs similar to scAAV genomes (FIG. 22C). SMRT sequencing confirmed the presence of these self-complimentary AAV genomes (FIGS. 23A-23B). This novel class of rAAVs is termed short-hairpin AAVs (shAAVs).

shAAV vectors were packaged with AAV9 capsid and administrated intravenously to adult mice. The three constructs that harbor shDNA sequences inserted between the CB promoter and the EGFP transgene (U6-shFluc1.3, H1-shApob1.3, and H1-shApob1.5 shAAV) were package shAAV genomes that lack the promoter for EGFP expression. Animals treated with these vectors produced few EGFP positive cells in the liver (FIG. 22D). While the H1-shApob2.0 shAAV vector achieved EGFP transduction at efficiency comparable to the transduction achieved by the scAAV-EGFP vector, the H1-shApob2.2 shAAV generated much less EGFP expression (FIG. 22D).

Southern blot analysis of total liver DNA showed that shAAV vector genomes persist as both linear and circular forms, similar to scAAV vectors in vivo (FIG. 22E). Interestingly, a dominant portion of shAAV-H1-shApob2.2 vector genomes was linear (FIG. 22E). This result indicates that circular shAAV genomes are primarily responsible for in vivo transduction and linear shAAV genomes are less potent and/or stable, which could explain the poor EGFP expression in the shAAV H1-shApob2.2 treated livers (FIG. 22D). In summary, by mimicking the mTR, shDNA sequences can generate intra-molecular double stranded genomes similar to classical rAAV vectors to produce novel shAAVs with the capacity for in vivo gene transfer.

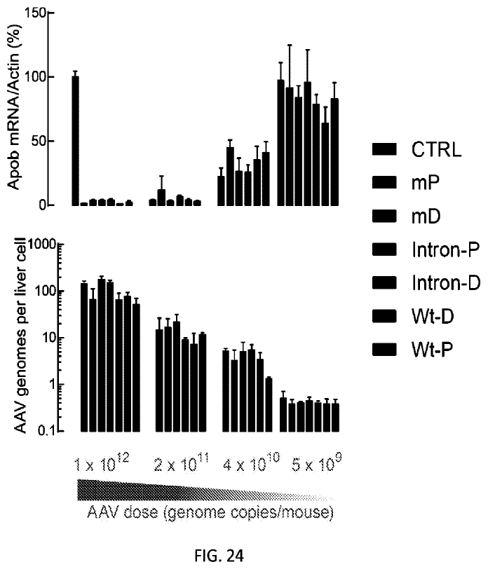

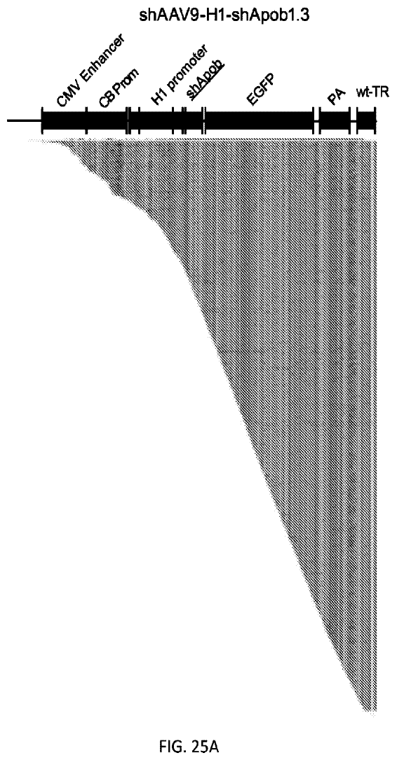

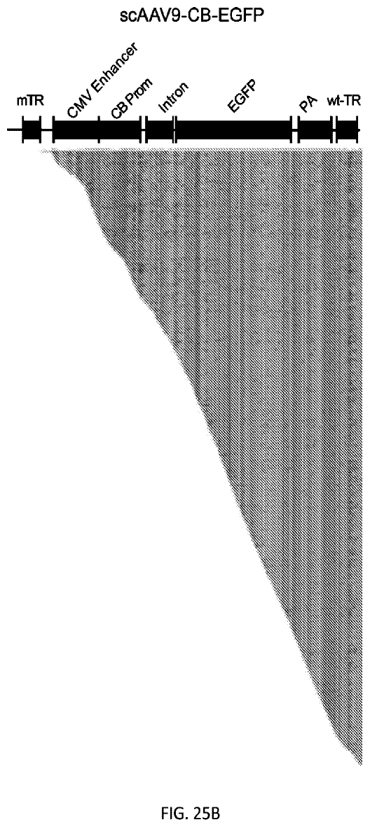

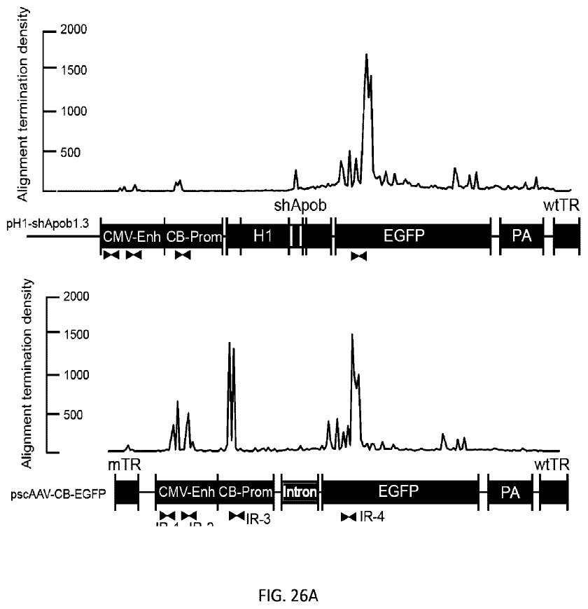

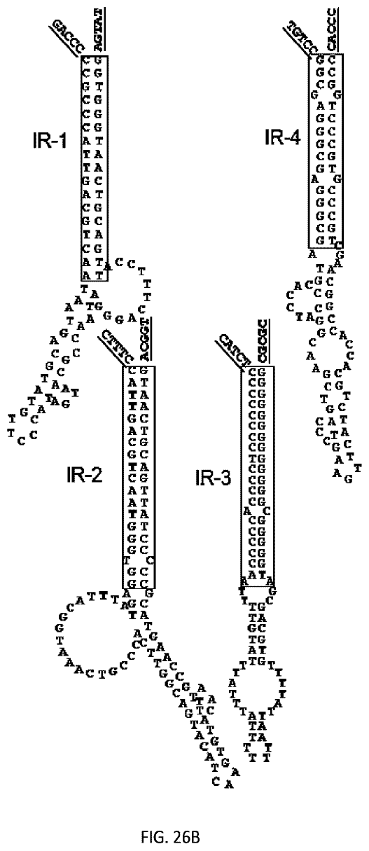

Unexpectedly, it was observed that ApoB gene expression was reduced in the livers of mice receiving shAAV vectors that carry shRNA cassettes targeting Apob (FIG. 22F). Gene silencing by shAAV vectors was unexpected, because SMRT sequencing data showed that these two shAAV vectors lack intact shApoB expression cassettes FIGS. 23A-23B). To validate SMRT sequencing results and to identify vector genomes that contained intact shRNA cassettes, the vector genomes of the H1-shApob1.3 and H1-shApob 1.5 constructs were analyzed by diagnostic enzymatic digestion using BglII (single cutter between H1 promoter and the sense strand of shDNA) or PstI (single cutter after the five-thymine termination signal) (FIG. 22G). Bands of 2.6-kb and 3.0-kb were detected for H1-shApob1.3 and H1-shApob 1.5 genomes, respectively. These bands represent the denatured intra-molecular double-stranded DNA genomes (FIG. 22G). SMRT sequencing data indicates that PstI digestion of H1-shApob 1.3 genomes removes the shDNA loop and results in ˜1.3 kb DNA fragments with open ends, while BglII should not cut in the H1-shApob1.3 genome (FIGS. 23A-23B). However, an additional fragment (>1.3 kb) was observed with BglII digestion, indicating the presence of vector genomes carrying the BglII site within the vector, and the successful replication through the shDNA sequence (arrow 1 in FIG. 22G). To substantiate the presence of such genomes, the vector DNA was digested with BstBI, which has a recognition site within the 5′-end of the H1 promoter. This treatment resulted in a reduction of the 3.0-kb band and an appearance of a new 1.5-kb band (arrow 2 in FIG. 22G). Together, this set of data indicates that packaged H1-Apob1.3 genomes are a mixture of vectors that possess intact H1-promoter-shRNA expression cassettes (˜25%), and shAAV genomes that lack functional shRNA expression cassettes (˜75%). A similar distribution of intact (˜35%, purple arrow in FIG. 5g) and incomplete (˜65%) shRNA cassettes among shAAV9-H1-shApob1.5 genomes (FIG. 22G) was also observed. Data indicate that despite the high prevalence of truncation events as a consequence of shRNA cassettes within rAAV genomes, a portion of genomes still harbor intact sequences as a result of complete replication through shDNA sequences (FIG. 22H). These “read-through” genomes generate enough functional shRNA to silence target gene expression, compensating the loss of RNAi functions from truncated genomes (FIG. 24).

The H1 or U6 promoter from the shAAV constructs (FIG. 221) and characterized these constructs in mouse livers. Comparable EGFP expression was only seen in the livers treated with shApob2.0, shApob2.OR, and control vectors (FIG. 22J). Neither the reduction of ApoB gene expression nor shRNA transcripts was detected in these livers (FIG. 22K), indicating that the complete shRNA expression cassette is necessary for functional silencing of Apob. This data also demonstrates that shDNA sequences alone, not other cassette elements, can promote the formation of shAAV genomes.

Other Hairpin-Like Sequences in rAAV Constructs Also Can Also Generate Intra-Molecular Double-Stranded Genomes

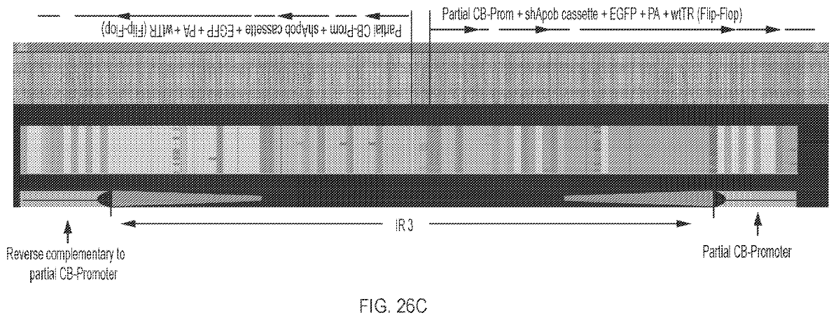

The prevalence of read-through genomes in purified vectors was investigated. Vector genomes were profiled by direct SMRT sequencing of shAAV9-H1-shApob1.3 vectors, followed by alignment to the pH1-shApob1.3 plasmid construct. To determine the abundance of read-through genomes as well as define the exact locations of genome truncation with high confidence, only full and intact alignments that span the wtTR region were considered (FIG. 25). It is notable that in addition to the previously identified shAAV genomes, several read-through genomes were identified. A significant portion of these genomes represent vectors that have replicated beyond the shRNA cassette, but terminate at the CMV enhancer or CB promoter regions as intra-molecular double-stranded DNAs, similar to shAAV genomes (FIG. 26A). To tabulate truncation events along the H1-shApob1.3 vector, each alignment was converted to an alignment termination positional tag designated as the most 5′ nucleotide of the read-alignment (FIG. 26A, top trace). The most substantial peaks of alignment termination density were within the EGFP transgene, indicating that the majority of vector truncation events are centered at the EGFP transgene. This phenomenon was also observed for the scAAV9-CB-EGFP vector that lacks an shRNA cassette (FIG. 26A, bottom trace).

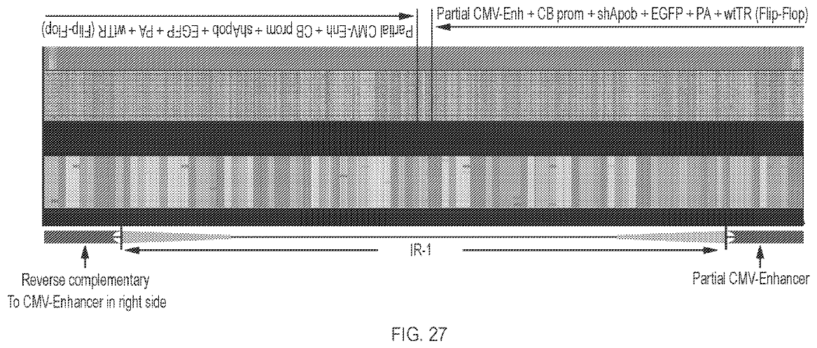

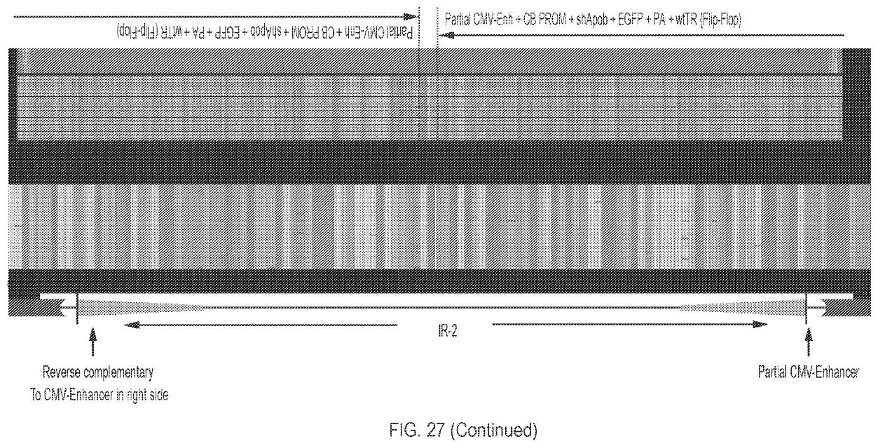

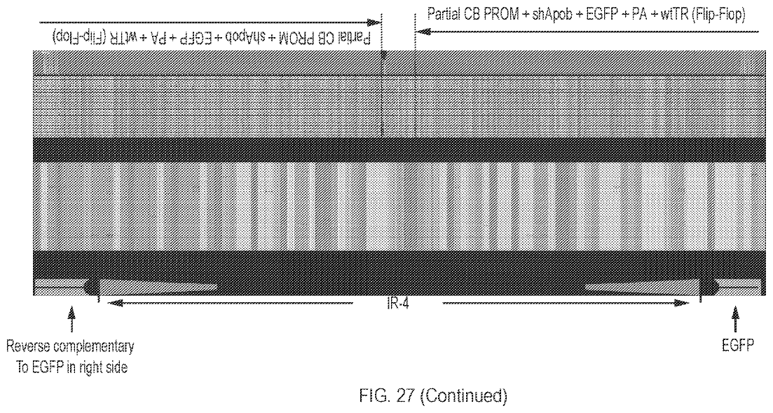

Four regions shared between the shAAV and the scAAV constructs with overlapping termination density peaks were identified and their secondary structures were analyzed: two within the CMV enhancer, one in the CB promoter, and one in the EGFP transgene (FIGS. 26A and 26B). Among these regions, inverted repeat (IR) sequences were identified. Custom references were designed using these inverted repeat sequences as centralized features, flanked by self-complementary strands as illustrated in FIG. 26C. Alignment of SMRT reads to these specific references verified our prediction that intra-molecular double-stranded genomes can also be mediated by sequences that harbor high secondary structure and inverted-repeat sequence (FIG. 26C and FIG. 27). These observations explain how constructs that only carry single wtTR regions and void of mTR or shDNA sequences can be rescued and packaged (FIG. 22B and FIG. 22C). The shDNA-like sequences inherent to the test vectors (e.g., CMV enhancer, CB promoter, and EGFP gene) function as pseudo-mTRs to complete genome replication. However, these shDNA-like sequences can also compromise promoter and transgene functionality in rAAV genomes, leading to low transgene expression in mice (FIG. 20D).

### Example 5: rAAV-Based Pri-miRNA Scaffolds Driven by Pol II Promoter to Inhibit Gene Expression

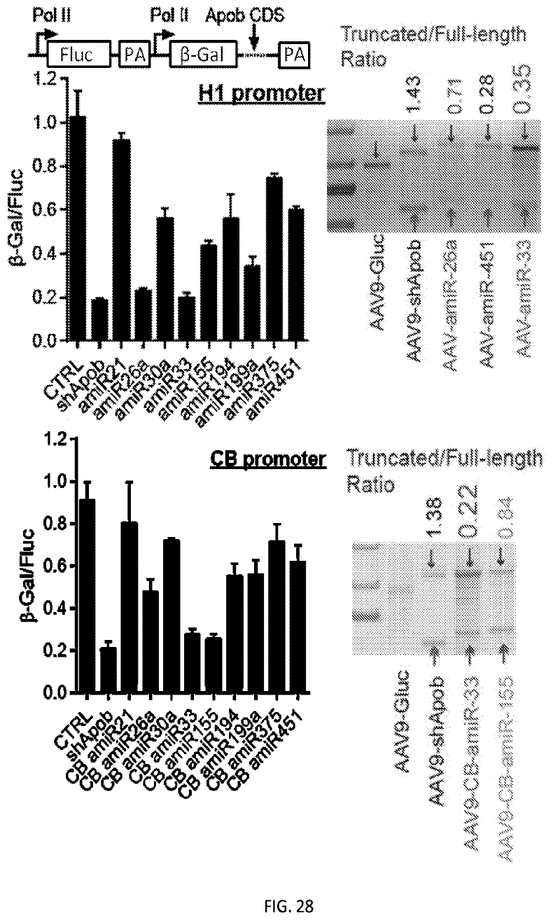

Here, rAAV-based pri-miRNA scaffolds driven by Pol II promoter are described. Highly efficient gene silencing was observed from artificial miRNA scaffolds driven by Pol II CMV enhancer/Chicken β-actin promoter (CB), compared to conventional shRNA driven by Pol III H1 promoter (FIG. 28). Improvements to the genomic integrity of rAAV vectors expressing small RNAs by pri-mmu-miR-33 based scaffold (FIG. 28, bottom) have been identified. Switching from the strong constitutive H1 promoter to Pol II promoter enables the approach of AAV delivered small silencing RNA to be regulated and safer in in vivo gene transfer. The Pol II AAV constructs, in some embodiments, achieve greater in vivo gene delivery by minimizing the truncated genomes and transgene expression can be inducible by chemicals or regulated by cell-type specific Pol II promoters.

### BRIEF DESCRIPTION OF SEQUENCE LISTING

| Sequence Reference | SEQ ID NO: |
|---|---|
| >pAAVsc\CB6\PI\EGFP\H1\apobsh3\(intron,\5′-3′) | 1 |
| >pAAVsc\CB6\PI\EGFP | 2 |
| >pAAVsc\CB\PI\EGFP\ApoBsh3\intron\(3′-5′)DmutITR | 3 |
| >pAAVsc\CB\PI\EGFP\ApoBsh3\intron\(5′-3′)DmutITR | 4 |
| >pAAVsc\CB6\ApoBsh3\(5′)(5′-3′)\EGFP\DmutITR | 5 |
| >pAAVsc\CB6\ApoBsh3\(5′)(3′-5′)\EGFP\DmutITR | 6 |
| >pAAVsc\CB\PI\EGFP\DmutantITR | 7 |
| >pAAVsc\CB\PI\EGFP\ApoBsh3\(3′)(5′-3′)DwtITR standard; circular DNA | 8 |
| >pAAVsc\CB6\siFluc\(intron)(5′-3′)\EGFP\DmutITR standard; circular DNA | 9 |
| >pAAVsc\CB6\siFluc\(intron)(5′-3′)\EGFP\DmutITR | 10 |
| >pAAVsc\CB6\ApoBsh3\(5′)(5′-3′)\EGFP\DmutITR\TshPC1 | 11 |
| >pAAVsc\CB\PI\EGFP\ApoBsh3\intron\(5′-3′)DmutITR\WtITRLoop | 12 |
| >pAAVsc\CB\PI\EGFP\ApoBsh3\intron\(5′-3′)DmutITR\TshApob | 13 |
| >pAAVsc\CB\PI\EGFP\ApoBsh3\intron\(5′-3′)DmutITR\TshPC1 | 14 |
| >pAAVsc\CB6\ApoBsh3\(5′)(5′-3′)\EGFP\DmutITR\WtITRLoop | 15 |
| >pAAVsc\CB6\ApoBsh3\(5′)(5′-3′)\EGFP\DmutITR\TshApob | 16 |
| >pAAVsc\CB6\siFluc\(intron)(5′-3′)\EGFP\DmutITR\WtITRLoop | 17 |
| >pAAVsc\CB6\siFluc\(intron)(5′-3′)\EGFP\DmutITR\T-shApob | 18 |
| >pAAVsc\CB6\siFluc\(intron)(5′-3′)\EGFP\DmutITR\T-shPC1 | 19 |
| Apobsensor-F | 20 |
| Apobsensor-R | 21 |
| Apob-F | 22 |
| Apob-R | 23 |
| Actin-F | 24 |
| Actin-R | 25 |
| Intron-R | 26 |
| PA-F | 27 |
| EGFP-F | 28 |
| EGFP-R | 29 |
| EGFP-probe | 30 |
| shApob AS probe | 31 |
| U6 probe | 32 |
| shApob | 33 |
| shFluc | 34 |

This disclosure is not limited in its application to the details of construction and the arrangement of components set forth in this description or illustrated in the drawings. The disclosure is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having thus described several aspects of at least one embodiment of this disclosure, it is to be appreciated various alterations, modifications, and improvements will readily occur to those skilled in the art. Such alterations, modifications, and improvements are intended to be part of this disclosure, and are intended to be within the spirit and scope of the disclosure. Accordingly, the foregoing description and drawings are by way of example only.

1. An rAAV vector comprising a single-stranded self-complementary nucleic acid with inverted terminal repeats (ITRs) at each of two ends and an inner portion comprising a hairpin-forming nucleic acid.

2.-18. (canceled)

19. An isolated nucleic acid having one inverted terminal repeat at a first terminus and a promoter operably linked with a sequence encoding a hairpin-forming RNA at a second terminus, wherein the isolated nucleic acid is configured for forming a self-complementary AAV (scAAV) vector.

20.-46. (canceled)

47. A preparation comprising a plurality of rAAVs, wherein at least 80% of the rAAVs comprise a non-truncated genome comprising a sequence encoding an artificial miRNA (AmiRNA), wherein the rAAV comprises the isolated nucleic acid of claims 19.

48.-51. (canceled)

52. A self-complementary adeno-associated virus (scAAV) comprising:
(i) a viral genome comprising a nucleic acid sequence encoding at least one inverted terminal repeat and a promoter operably linked with a nucleic acid sequence encoding a hairpin-forming RNA; and
(ii) at least one AAV capsid protein serotype.

53.-69. (canceled)

70. A host cell comprising the rAAV vector of claim 1.

71. A host cell comprising the isolated nucleic acid of claim 19.

72. A host cell comprising the scAAV of claim 52.

73.-76. (canceled)

77. A pharmaceutical composition comprising the rAAV vector of claims 1 and a pharmaceutically acceptable carrier.

78. A pharmaceutical composition comprising the isolated nucleic acid of claim 19 and a pharmaceutically acceptable carrier.

79. A pharmaceutical composition comprising the scAAV of claim 52 and a pharmaceutically acceptable carrier

80. A method of treating metabolic disorders, immune disorders, cancers, inflammatory disorders, muscular dystrophies, central nervous system diseases, and/or diseases caused by gain of function mutations,
the method comprising administering the subject the preparation of claims 47.

81. A method of treating metabolic disorders, immune disorders, cancers, inflammatory disorders, muscular dystrophies, central nervous system diseases, and/or diseases caused by gain of function mutations,
the method comprising administering the subject the pharmaceutical composition of claims 78.

82. A method of treating metabolic disorders, immune disorders, cancers, inflammatory disorders, muscular dystrophies, central nervous system diseases, and/or diseases caused by gain of function mutations,
the method comprising administering the subject the pharmaceutical composition of claims 79.

83. A method of producing a preparation of rAAV, the method comprising delivering to a host cell:
the isolated nucleic acid of claim 19,
an AAV helper function vector encoding a Rep protein and a Cap protein; and
an AAV accessory function vector.

84. The method of claim 83, wherein at least 80% of the rAAVs in the preparation comprise a non-truncated genome.
